SEQUENCE LISTING (100 sequences)

SEQ ID NO: 1 - protein, 4 aa, artificial sequence
  therapeutic agent
YDHF

SEQ ID NO: 2 - protein, 4 aa, artificial sequence
  therapeutic agent
YERF

SEQ ID NO: 3 - protein, 4 aa, artificial sequence
  therapeutic agent
YEHF

SEQ ID NO: 4 - protein, 4 aa, artificial sequence
  therapeutic agent
WDHF

SEQ ID NO: 5 - protein, 4 aa, artificial sequence
  therapeutic agent
WEHF

SEQ ID NO: 6 - protein, 4 aa, artificial sequence
  therapeutic agent
YDRF

SEQ ID NO: 7 - protein, 4 aa, artificial sequence
  therapeutic agent
YDKF

SEQ ID NO: 8 - protein, 4 aa, artificial sequence
  therapeutic agent
YEKF

SEQ ID NO: 9 - protein, 4 aa, artificial sequence
  therapeutic agent
WEKF

SEQ ID NO: 10 - protein, 4 aa, artificial sequence
  therapeutic agent
WERF

SEQ ID NO: 11 - protein, 4 aa, artificial sequence
  therapeutic agent
WDKF

SEQ ID NO: 12 - protein, 4 aa, artificial sequence
  therapeutic agent
WDRF

SEQ ID NO: 13 - protein, 4 aa, artificial sequence
  therapeutic agent
YDHW

SEQ ID NO: 14 - protein, 4 aa, artificial sequence
  therapeutic agent
YEHW

SEQ ID NO: 15 - protein, 4 aa, artificial sequence
  therapeutic agent
WDHW

SEQ ID NO: 16 - protein, 4 aa, artificial sequence
  therapeutic agent
WEHW

SEQ ID NO: 17 - protein, 4 aa, artificial sequence
  therapeutic agent
YDRW

SEQ ID NO: 18 - protein, 4 aa, artificial sequence
  therapeutic agent
YDKW

SEQ ID NO: 19 - protein, 4 aa, artificial sequence
  therapeutic agent
YEKW

SEQ ID NO: 20 - protein, 4 aa, artificial sequence
  therapeutic agent
YERW

SEQ ID NO: 21 - protein, 4 aa, artificial sequence
  therapeutic agent
WEKW

SEQ ID NO: 22 - protein, 4 aa, artificial sequence
  therapeutic agent
WERW

SEQ ID NO: 23 - protein, 4 aa, artifical sequence
WDKW

SEQ ID NO: 24 - protein, 4 aa, artifical sequence
WDRW

SEQ ID NO: 25 - protein, 4 aa, artificial sequence
  therapeutic agent
YDHY

SEQ ID NO: 26 - protein, 4 aa, artificial sequence
  therapeutic agent
  MISC_FEATURE (1)..(4): residues 1 - 4 are D-amino acids
YERF

SEQ ID NO: 27 - protein, 4 aa, artificial sequence
  therapeutic agent
  MISC_FEATURE (1)..(4): residues 1 - 4 are D-amino acids
YDHF

SEQ ID NO: 28 - protein, 4 aa, artifical sequence
  MISC_FEATURE (1)..(4): residues 1 - 4 are D-amino acids
WERF

SEQ ID NO: 29 - protein, 4 aa, artificial sequence
  therapeutic agent
  MISC_FEATURE (1)..(4): residues 1 - 4 are D-amino acids
WDHF

SEQ ID NO: 30 - protein, 4 aa, artificial sequence
  therapeutic agent
  misc_feature (1)..(4): residues 1 - 4 are D-amino acids
YDRF

SEQ ID NO: 31 - protein, 4 aa, artificial sequence
  therapeutic agent
  MISC_FEATURE (1)..(4): residues 1 - 4 are D-amino acids
YDHY

SEQ ID NO: 32 - protein, 4 aa, artificial sequence
  therapeutic agent
  MISC_FEATURE (1)..(4): residues 1 - 4 are D-amino acids
YERY

SEQ ID NO: 33 - protein, 4 aa, artificial sequence
  therapeutic agent
  MISC_FEATURE (1)..(4): residues 1 - 4 are D-amino acids
WDHY

SEQ ID NO: 34 - protein, 4 aa, artificial sequence
  therapeutic agent
  MISC_FEATURE (1)..(4): residues 1 - 4 are D-amino acids
WERY

SEQ ID NO: 35 - protein, 4 aa, artificial sequence
  therapeutic agent
  MISC_FEATURE (1)..(4): residues 1 - 4 are D-amino acids
YDKF

SEQ ID NO: 36 - protein, 4 aa, artificial sequence
  therapeutic agent
  MISC_FEATURE (1)..(4): residues 1 - 4 are D-amino acids
YEHF

SEQ ID NO: 37 - protein, 4 aa, artificial sequence
  therapeutic agent
  MISC_FEATURE (1)..(4): residues 1 - 4 are D-amino acids
YDKF

SEQ ID NO: 38 - protein, 4 aa, artificial sequence
  therapeutic agent
  MISC_FEATURE (1)..(4): residues 1 - 4 are D-amino acids
WEHF

SEQ ID NO: 39 - protein, 4 aa, artificial sequence
  therapeutic agent
  MISC_FEATURE (1)..(4): residues 1 - 4 are D-amino acids
YEKF

SEQ ID NO: 40 - protein, 4 aa, artificial sequence
  therapeutic agent
  MISC_FEATURE (1)..(4): residues 1 - 4 are D-amino acids
WEKF

SEQ ID NO: 41 - protein, 4 aa, artificial sequence
  therapeutic agent
  MISC_FEATURE (1)..(4): residues 1 - 4 are D-amino acids
WDKF

SEQ ID NO: 42 - protein, 4 aa, artificial sequence
  therapeutic agent
  MISC_FEATURE (1)..(4): residues 1 - 4 are D-amino acids
YDHW

SEQ ID NO: 43 - protein, 4 aa, artificial sequence
  therapeutic agent
  MISC_FEATURE (1)..(4): residues 1 - 4 are D-amino acids
YEHW

SEQ ID NO: 44 - protein, 4 aa, artificial sequence
  therapeutic agent
  MISC_FEATURE (1)..(4): residues 1 - 4 are D-amino acids
WDHW

SEQ ID NO: 45 - protein, 4 aa, artificial sequence
  therapeutic agent
  MISC_FEATURE (1)..(4): residues 1 - 4 are D-amino acids
WEHW

SEQ ID NO: 46 - protein, 4 aa, artificial sequence
  therapeutic agent
  MISC_FEATURE (1)..(4): residues 1 - 4 are D-amino acids
YDRW

SEQ ID NO: 47 - protein, 4 aa, artificial sequence
  therapeutic agent
  MISC_FEATURE (1)..(4): residues 1 - 4 are D-amino acids
YDKW

SEQ ID NO: 48 - protein, 4 aa, artificial sequence
  therapeutic agent
  MISC_FEATURE (1)..(4): residues 1 - 4 are D-amino acids
YEKW

SEQ ID NO: 49 - protein, 4 aa, artificial sequence
  therapeutic agent
  MISC_FEATURE (1)..(4): residues 1 - 4 are D-amino acids
YERW

SEQ ID NO: 50 - protein, 4 aa, artificial sequence
  therapeutic agent
  MISC_FEATURE (1)..(4): residues 1 - 4 are D-amino acids
WEKW

SEQ ID NO: 51 - protein, 4 aa, artificial sequence
  therapeutic agent
  MISC_FEATURE (1)..(4): residues 1 - 4 are D-amino acids
WERW

SEQ ID NO: 52 - protein, 4 aa, artificial sequence
  therapeutic agent
  MISC_FEATURE (1)..(4): residues 1 - 4 are D-amino acids
WDKW

SEQ ID NO: 53 - protein, 4 aa, artificial sequence
  therapeutic agent
  MISC_FEATURE (1)..(4): residues 1 - 4 are D-amino acids
WQRW

SEQ ID NO: 54 - protein, 4 aa, artificial sequence
  therapeutic agent
  MISC_FEATURE (1)..(4): residues 1 - 4 are D-amino acids
WNKW

SEQ ID NO: 55 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
YERF

SEQ ID NO: 56 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
YDHF

SEQ ID NO: 57 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
YDKF

SEQ ID NO: 58 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (1)..(1): AMIDATION
YEKF

SEQ ID NO: 59 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
YEHF

SEQ ID NO: 60 - protein, 4 aa, artificial sequence therapeutic agent
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
YDRF

SEQ ID NO: 61 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
WEHF

SEQ ID NO: 62 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
WEKF

SEQ ID NO: 63 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
WDHF

SEQ ID NO: 64 - protein, 4 aa, artificial sequence
  therapeutic agent
  MISC_FESATURE (1)..(4): D or L amino acid configuration not specified. Both configurations encompassed.
  MOD_RES (1)..(1): ACETYLATION
  MOD_RES (4)..(4): AMIDATION
WDKF

SEQ ID NO: 65 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
YERY

SEQ ID NO: 66 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
YDKY

SEQ ID NO: 67 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
YEKY

SEQ ID NO: 68 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
YEHY

SEQ ID NO: 69 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
YDRY

SEQ ID NO: 70 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
WEHY

SEQ ID NO: 71 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
WEKY

SEQ ID NO: 72 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
WDHY

SEQ ID NO: 73 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
WDKY

SEQ ID NO: 74 - protein, 4 aa, artificial sequence
  therapeutic agent
  MISC_FEATURE (1)..(4): D or L amino acid configuration not specified. Both configurations encompassed.
  MISC_FEATURE (1)..(4): INTERNAL LACTAM
  MOD_RES (1)..(1): ACETYLATION
  MOD_RES (4)..(4): AMIDATION
YEKF

SEQ ID NO: 75 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
YQRF

SEQ ID NO: 76 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
YMRF

SEQ ID NO: 77 - protein, 4 aa, artificial sequence
  therapeutic agent
  MISC_FEATURE (1)..(4): D or L amino acid configuration not specified. Both configurations encompassed.
  MOD_RES (1)..(1): ACETYLATION
  MOD_RES (4)..(4): AMIDATION
YLRF

SEQ ID NO: 78 - protein, 4 aa, artificial sequence
  therapeutic agent
  MISC_FEATURE (1)..(4): D or L amino acid configuration not specified. Both configurations encompassed.
  MOD_RES (1)..(1): Benzyloxycarbonyl-Tyr
  MOD_RES (4)..(4): AMIDATION
YERF

SEQ ID NO: 79 - protein, 4 aa, artificial sequence
  therapeutic agent
  MISC_FEATURE (1)..(4): D or L amino acid configuration not specified. Both configurations encompassed.
  MOD_RES (1)..(1): Benzyloxycarbonyl-Tyr
  MOD_RES (4)..(4): AMIDATION
YDHF

SEQ ID NO: 80 - protein, 4 aa, artificial sequence
  therapeutic agent
  MISC_FEATURE (1)..(4): D or L amino acid configuration not specified. Both configurations encompassed.
  MOD_RES (1)..(1): Benzyloxycarbonyl-Tyr
  MOD_RES (2)..(2): 4-methyl ester aspartic acid (Asp(OMe))
  MOD_RES (4)..(4): AMIDATION
YDHF

SEQ ID NO: 81 - protein, 4 aa, artificial sequence
  therapeutic agent
  MISC_FEATURE (1)..(4): D or L amino acid configuration not specified. Both configurations encompassed.
  MOD_RES (1)..(1): (3-Methoxy,4-hydroxy-benzoyl)-Tyr
  MOD_RES (4)..(4): AMIDATION
YERF

SEQ ID NO: 82 - protein, 4 aa, artificial sequence
  therapeutic agent
  MISC_FEATURE (1)..(4): D or L amino acid configuration not specified. Both configurations encompassed.
  MOD_RES (1)..(1): (3-Methoxy,4-hydroxy-benzoyl)-Tyr
  MOD_RES (4)..(4): AMIDATION
YDHF

SEQ ID NO: 83 - protein, 4 aa, artificial sequence
  therapeutic agent
  MISC_FEATURE (1)..(4): D or L amino acid configuration not specified. Both configurations encompassed.
  MOD_RES (1)..(1): N-(3-Methoxy,4-hydroxy-benzoyl)ation
  MOD_RES (2)..(2): 4-methyl ester aspartic acid  (Asp(OMe))
  MOD_RES (4)..(4): AMIDATION
YDHF

SEQ ID NO: 84 - protein, 4 aa, artificial sequence
  therapeutic agent
  MISC_FEATURE (1)..(4): D or L amino acid configuration not specified. Both configurations encompassed.
  MOD_RES (1)..(1): Fluorenylmethyloxycarbonyl-Tyr
  MOD_RES (4)..(4): AMIDATION
YERF

SEQ ID NO: 85 - protein, 4 aa, artificial sequence
  therapeutic agent
  MISC_FEATURE (1)..(4): D or L amino acid configuration not specified. Both configurations encompassed.
  MOD_RES (1)..(1): Fluorenylmethyloxycarbonyl-Tyr
  MOD_RES (4)..(4): AMIDATION
YDHF

SEQ ID NO: 86 - protein, 4 aa, artificial sequence
  therapeutic agent
  MISC_FEATURE (1)..(4): D or L amino acid configuration not specified. Both configurations encompassed.
  MOD_RES (1)..(1): Fluorenylmethyloxycarbonyl-Tyr
  MOD_RES (2)..(2): 4-methyl ester aspartic acid (Asp(OMe))
  MOD_RES (4)..(4): AMIDATION
YDHF

SEQ ID NO: 87 - protein, 4 aa, artificial sequence
  therapeutic agent
  MISC_FEATURE (1)..(4): D or L amino acid configuration not specified. Both configurations encompassed.
  MOD_RES (1)..(1): Fluorenylmethyloxycarbonyl-Tyr
  MOD_RES (2)..(2): 4-methyl ester aspartic acid (Asp(OMe))
  MOD_RES (4)..(4): AMIDATION
YDHF

SEQ ID NO: 88 - protein, 4 aa, artificial sequence
  therapeutic agent
  MISC_FEATURE (1)..(4): D or L amino acid configuration not specified. Both configurations encompassed.
  MOD_RES (1)..(1): Myristyl-Tyr
  MOD_RES (4)..(4): AMIDATION
YERF

SEQ ID NO: 89 - protein, 4 aa, artificial sequence
  therapeutic agent
  MISC_FEATURE (1)..(4): D or L amino acid configuration not specified. Both configurations encompassed.
  MOD_RES (1)..(1): Myristyl-Tyr
  MOD_RES (4)..(4): AMIDATION
YDHF

SEQ ID NO: 90 - protein, 4 aa, artificial sequence
  therapeutic agent
  MISC_FEATURE (1)..(4): D or L amino acid configuration not specified. Both configurations encompassed.
  MOD_RES (1)..(1): Myristyl-Tyr
  MOD_RES (2)..(2): 4-methyl ester aspartic acid
  MOD_RES (4)..(4): AMIDATION
YDHF

SEQ ID NO: 91 - protein, 9 aa, artificial sequence
  therapeutic agent
  MISC_FEATURE (1)..(9): D or L amino acid configuration not specified. Both configurations encompassed.
  MOD_RES (1)..(1): ACETYLATION
  MOD_RES (9)..(9): AMIDATION
YERFGYERF

SEQ ID NO: 92 - protein, 9 aa, artificial sequence
  therapeutic agent
  MISC_FEATURE (1)..(9): D or L amino acid configuration not specified. Both configurations encompassed.
  MOD_RES (1)..(1): ACETYLATION
  MOD_RES (9)..(9): AMIDATION
YDHFGYDHF

SEQ ID NO: 93 - protein, 7 aa, artificial sequence
  therapeutic agent
  MISC_FEATURE (1)..(7): D or L amino acid configuration not specified. Both configurations encompassed.
  MOD_RES (1)..(1): ACETYLATION
  MOD_RES (7)..(7): AMIDATION
YRFGYRF

SEQ ID NO: 94 - protein, 9 aa, artificial sequence
  therapeutic agent
  MISC_FEATURE (1)..(9): D or L amino acid configuration not specified. Both configurations encompassed.
  MOD_RES (1)..(1): ACETYLATION
  MOD_RES (2)..(2): 4-methyl ester aspartic acid
  MOD_RES (7)..(7): 4-methyl ester aspartic acid
  MOD_RES (9)..(9): AMIDATION
YDHFGYDHF

SEQ ID NO: 95 - protein, 9 aa, artificial sequence
  therapeutic agent
  MISC_FEATURE (1)..(9): D or L amino acid configuration not specified. Both configurations encompassed.
  MOD_RES (1)..(1): benzyloxycarbonyl-Tyr
  MOD_RES (9)..(9): AMIDATION
YERFGYERF

SEQ ID NO: 96 - protein, 9 aa, artificial sequence
  therapeutic agent
  MISC_FEATURE (1)..(9): D or L amino acid configuration not specified. Both configurations encompassed.
  MOD_RES (1)..(1): benzyloxycarbonyl-Tyr
  MOD_RES (9)..(9): AMIDATION
YDHFGYDHF

SEQ ID NO: 97 - protein, 7 aa, artificial sequence
  therapeutic agent
  MISC_FEATURE (1)..(7): D or L amino acid configuration not specified. Both configurations encompassed.
  MOD_RES (1)..(1): benzyloxycarbonyl-Tyr
  MOD_RES (7)..(7): AMIDATION
YRFGYRF

SEQ ID NO: 98 - protein, 9 aa, artificial sequence
  therapeutic ageny
  MISC_FEATURE (1)..(9): D or L amino acid configuration not specified. Both configurations encompassed.
  MOD_RES (1)..(1): benzyloxycarbonyl-Tyr
  MOD_RES (2)..(2): 4-methyl ester aspartic acid
  MOD_RES (7)..(7): 4-methyl ester aspartic acid
  MOD_RES (9)..(9): AMIDATION
YDHFGYDHF

SEQ ID NO: 99 - protein, 5 aa, artificial sequence
  cleavable linker
DDDDY

SEQ ID NO: 100 - protein, 5 aa, artificial sequence
  cleavable linker
HPFHL

(19) United States
(12) Patent Application Publication
(10) Pub. No.: US 2017/0240592 A1
(43) Pub. Date: Aug. 24, 2017

(54) GADD45BETA/MKK7 INHIBITOR FOR THE TREATMENT OF A RESISTANT HAEMATOLOGICAL MALIGNANCY

(71) Applicant: IMPERIAL INNOVATIONS LIMITED, London, Greater London (GB)

(72) Inventors: Guido FRANZOSO, London (GB); Laura TORNATORE, London (GB); Federica BEGALLI, London (GB); Daniel D'ANDREA, London (GB)

(21) Appl. No.: 15/503,522

(22) PCT Filed: Aug. 14, 2015
(86) PCT No.: PCT/GB2015/052366

§ 371 Date: Feb. 13, 2017

(30) Foreign Application Priority Data
Aug. 15, 2014 (GB) 1414542.9

(51) Int. Cl. C07K 5/087; A61K 31/69; C07K 5/107; A61K 38/07; A61K 45/06; A61K 38/06

(52) U.S. Cl. CPC C07K 5/0812; A61K 45/06; A61K 38/06; C07K 5/1016; A61K 38/07; A61K 31/69

(57) ABSTRACT

A Gadd45β/MKK7 inhibitor for use in a method of treating a resistant haematological malignancy, combinations of said inhibitor with a further anti-cancer agent and related methods of use.

### FIELD OF INVENTION

The present invention relates to methods for treating haematological malignancies using Gadd45β/MKK7 inhibitors. In particular, the invention relates to the treatment of haematological malignancies which are resistant to treatment with other anti-cancer agents. The invention also relates to specific combinations of a Gadd45β/MKK7 inhibitor and a further anti-cancer agent.

### BACKGROUND OF THE INVENTION

There are a number of cellular pathways involved in carcinogenesis and cancer progression including the c-Jun N-terminal kinase JNK pathway. JNKs are responsive to cytokines and stress stimuli such as ultraviolet irradiation, heat shock and osmotic shock. Also activated in the response to cytokines and cellular stress is the NF-κB pathway. Significant effort has been expended by the pharmaceutical industry in developing specific NF-κB or IKKβ inhibitors for indication both within and outside of oncology. However, there are toxicities associated with the global suppression of NF-κB (Di Donato et al., 2012) and, as a result, it may be preferred to inhibit specific functions of NF-κB rather than NF-κB itself. The NF-κB pathway can inhibit the JNK pathway by crosstalk mediated by Gadd45β and the JNK kinase, mitogen activated protein-kinase kinase 7 (MKK7/JNKK2). MKK7 activity is inhibited by Gadd45β, a member of the Gadd45 family of inducible factors and a direct transcriptional target of NF-κB. This means that Gadd45β mediates NF-κB suppression of JNK signalling by binding to MKK7 and inhibiting its activity. Papa, et al. 2004, Nature Cell Biology 6(2):1462153. Elevated levels of Gadd45β expression have been associated with an increased likelihood of having a haematological malignancy such as multiple myeloma (see for example WO2012/146940). A series of peptide compounds having activity as Gadd45β/MKK7 inhibitors has also been described (see WO2011/048390).

Multiple myeloma (MM), also known as plasma cell myeloma or Kahler's disease, is a cancer of plasma cells and is currently incurable. According to the American Cancer Society, there are approximately 45,000 people in the United States living with multiple myeloma with approximately 15,000 new cases being diagnosed each year in the United States. The average survival time from diagnosis is approximately five years. Multiple myeloma is the second most prevalent blood cancer after non-Hodgkin's lymphoma and represents approximately 1% of all cancers and approximately 2% of all cancer deaths. The incidence of multiple myeloma appears to be increasing and there is also some evidence that the age of onset of the disease is falling.

Current treatment of multiple myeloma includes chemotherapy and steroids combined with newer agents, such as proteasome inhibitors (e.g. bortezomib) and immunomodulatory drugs (IMiDs), whereas stem cell transplantation is an option for select patients. These treatments, however, generally achieve only temporary remissions, and so most patients eventually relapse and/or develop drug resistance (Rajkumar, 2011; Mahindra et al., 2012). Thus, despite the introduction of new treatments, the management of myeloma patients remains a major medical problem. There is a clear need for improved therapies for the treatment of multiple myeloma and other haematological malignancies.

The present invention is based on the discovery that Gadd45β/MKK7 inhibitors, such as DTP3

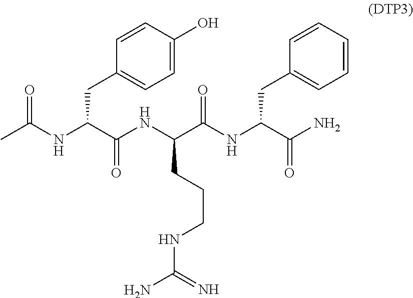 [SMILES: CC(=O)N[C@H](Cc1ccc(O)cc1)C(=O)N[C@H](CCCNC(=N)N)C(=O)N[C@H](Cc1ccccc1)C(N)=O]

are surprisingly effective therapeutic agents useful for the treatment of haematological malignancies such as multiple myeloma. The inventors have found that DTP3 retains full therapeutic efficacy against a range of multiple myeloma cell lines, including those which are resistant to current multiple myeloma treatments such as bortezomib, dexamethasone and lenalidomide, and is also effective against resistant diffuse large B-cell lymphoma (DLBCL) cell lines. Furthermore, the combination of DTP3 with bortezomib displays synergistic activity in different multiple myeloma cell lines, demonstrating that the combination of a Gadd45β/MKK7 inhibitor with an anti-cancer agent which acts via the NF-κB pathway but has a different mechanism of action is a particularly effective therapy. It has also been found that Gadd45β/MKK7 inhibitors such as DTP3 are highly selective for cancer cells, leading to improved therapies which are more efficacious and/or which have fewer side-effects.

### SUMMARY OF THE INVENTION

In a first aspect, the invention provides a Gadd45β/MKK7 inhibitor for use in a method of treating a resistant haematological malignancy in a subject, the method comprising the step of administering the Gadd45β/MKK7 inhibitor to the subject.

In a further aspect, the invention also provides a combination comprising i) DTP3, or a derivative thereof, or a salt thereof including a salt of a derivative thereof

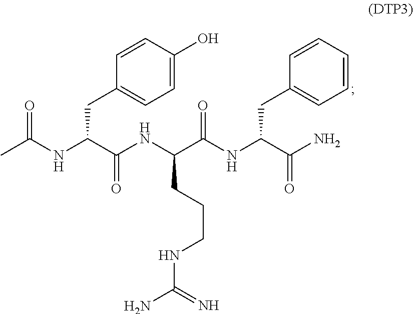 [SMILES: CC(=O)N[C@H](Cc1ccc(O)cc1)C(=O)N[C@H](CCCNC(=N)N)C(=O)N[C@H](Cc1ccccc1)C(N)=O]

and
ii) a further anti-cancer agent which is a proteasome inhibitor.

In a further aspect, the invention also provides a combination comprising i) DTP3, or a derivative thereof, or a salt thereof including a salt of a derivative thereof; and ii) a further anti-cancer agent which is an IMiD anti-cancer agent.

In a further aspect, the invention also provides a combination comprising i) DTP3, or a derivative thereof, or a salt thereof including a salt of a derivative thereof; and ii) a further anti-cancer agent which is a glucocorticoid.

### BRIEF DESCRIPTION OF THE DRAWINGS

FIG. 1 shows the results of [³H]thymidine incorporation assays showing the survival of U266, KMS-12, KMS-11, JJN-3, NCI-H929 and RPMI-8226 multiple myeloma cell lines after a 6-day treatment with the indicated concentrations of z-DTP1, z-DTP2, or Z-protected (z)-DNC. Values express the percentage of live cells in the treated cultures relative to the live cells in the respective untreated cultures, represented as 100%, and denote means±SD.

FIG. 2 shows the IC₅₀ values of z-DTP1 and z-DTP2 at 144 hr, as determined by [³H]thymidine incorporation assays, in genetically heterogeneous multiple myeloma cell lines that either depend or do not depend on Gadd45β for survival.

FIG. 3 shows the survival of healthy mouse splenocytes and lymph node (LN) cells after treatment with z-DTP1 or z-DTP2 for 72 hr. Cell viability was measured using [³H]thymidine incorporation assays. Values express the percentage of live cells present in the treated cultures relative to the live cells present in the respective untreated cultures, represented as 100%. UT, untreated.

FIG. 4 shows the results of [³H]thymidine incorporation assays showing the survival of Gadd45β-dependent (top 2 rows and left of bottom row) and Gadd45β-independent (bottom row, middle and right) multiple myeloma cell lines after a 6-day treatment with the indicated concentrations of DTP3 or a negative control D-peptide (z-DNC).

FIG. 5 shows the IC₅₀ values of DTP3 at 144 hr for the experiment shown in FIG. 4.

FIG. 6 shows trypan blue exclusion assays showing the survival of mouse LN cells and splenocytes after treatment with DTP3 (100 μM) or PS-1145 (20 μM) for 144 hr. Values express the percentage of live cells present in the treated cultures relative to the live cells present in the respective untreated cultures and denote means±SD ([WM]; n=3). UT, untreated.

FIG. 7 shows ELISA Gadd45β/MKK7 competition assays showing the IC₅₀ values of DTP3 and the scrambled control D-tripeptide, SCRB, before and after a 48-hr pre-incubation with human serum, at 37° C., as indicated. Values express the percentage of inhibition of Gadd45β binding to MKK7 relative to the binding measured in the absence of peptide.

FIG. 8 shows co-immunoprecipitation assays showing the disruption of the Gadd45β/MKK7 complex by DTP3, but not by the SCRB control D-peptide, at the indicated concentrations. Co-immunoprecipitations (IP) were performed using an anti-FLAG antibody (a-FLAG); western blots (WB) were developed using an anti-HA or an anti-MKK7 antibody, as shown. The first column marked “-” shows incubation without D-tripeptide.

FIG. 9 shows PI nuclear staining assays showing apoptotic cells in diffuse large B-cell lymphoma (DLBCL) cell lines (HT, SU-DHL-8, U-2932 and RC-K8) following treatment with 10 μM of either DTP3 or the scrambled control D-tripeptide, SCR for 6 days. The percentages of apoptotic cells are depicted.

FIG. 10 shows the results of [³H]thymidine incorporation assays showing the survival of sensitive and drug-resistant multiple myeloma cell lines following a 6-day treatment with the indicated concentrations of DTP3. Values express the percentage of the cpm measured with the treated cultures relative to the cpm measured with the respective untreated cultures and denote means±SD (n=3). Matching pairs of sensitive (parental) and drug-resistant multiple myeloma cell lines were as follows: MM1.S (parental) and MM1.R (dexamethasone-resistant); AMO-1 (parental) and AMO-1a (bortezomib-resistant); MM1.S (parental) and MM1/R10R (lenalidomide-resistant); U266 (parental) and U266/R10R (lenalidomide-resistant). Data with the parental U266 multiple myeloma cell line were from the experiment shown in FIGS. 4 and 5 (5 out of 7 DTP3 concentrations only). For clarity purposes, the same data for the parental MM1.S multiple myeloma cell line are shown as control for the dexamethasone-resistant (MM1.R) multiple myeloma cell line and the lenalidomide-resistant (MM1/R10R) multiple myeloma cell line.

FIG. 11 shows IC₅₀ values of DTP3 for the experiment shown in FIG. 10.

FIG. 12 shows the results of [³H]thymidine incorporation assays showing the survival of representative GADD45β-dependent multiple myeloma cell lines after treatment with the indicated concentrations of DTP3 and bortezomib, used either as single agents or in combination. Treatments with DTP3 were for 6 days; bortezomib was added to the cell cultures 48 hr prior to the measurement of cell viability. For the combination treatment, DTP3 was used at the concentrations of 3 nM in U266 cells and of 10 nM in KMS-12 cells, whereas bortezomib was used at increasing concentrations, as shown. Based on the data shown, the IC₅₀ value of bortezomib as single agent at 48 hr was 6 nM in each of the two multiple myeloma cell lines. The survival curves of U266 and KMS-12 cells following treatment with DTP3 as single agent are from the experiment shown in FIGS. 4 and 5.

FIG. 13 shows the combination index (CI) of DTP3 and bortezomib for the experiment shown in FIG. 12. Viability data from the experiment shown in FIG. 12 were converted into values representing the fraction of cells affected (FA) equaling 0.5 in the drug-treated cultures compared with untreated cultures, and the interaction of DTP3 with bortezomib was analysed according to the Chou-Talalay method (Chou, 2006). Also shown are the drug concentrations giving the FA value of 0.5 and the description of the CI. Based on the Chou-Talalay equation (Chou, 2006), synergy is present when the CI is less than 1.0 (where values between 0.3 and 0.7 denote synergism, and values between 0.1 and 0.3 denote strong synergism); the combination is additive when CI equals 1.0, and antagonistic when it is more than 1.0 (Chou, 2006).

FIG. 14 shows pharmacokinetic (PK) values of DTP3 after single intravenous injection at the dose of 10 mg/kg. t_{1/2}, terminal half-life; CL, plasma clearance; V_{d}, volume of distribution; AUC, area under the plasma concentration versus time curve. Values denote means±SD (n=3).

FIG. 15 shows the values of the main in vitro pharmacokinetic (PK) parameters of DTP3, including distribution coefficient (Log D), plasma stability, plasma protein binding (PPB), microsomal stability, and thermodynamic solubility. Also shown is the excellent tolerability of DTP3 in mice after either a single intravenous (i.v.) injection, a subcutaneous (s.c.) injection, or a per os administration at the doses indicated. Additionally, shown are the excellent tolerability and lack of any apparent side effects of DTP3 after prolonged administration via osmotic pumps over a period of 28 days at the therapeutic dose of 14.5 mg/kg/day. Reported at the bottom are the values of the main in-vivo pharmacokinetic parameters of z-DTP2 after a single intravenous injection at the dose of 10 mg/kg, including terminal half-life (t_{1/2}), plasma clearance (CL), volume of distribution (V_{d}), and area under the plasma concentration versus time curve (AUC). DTP3 exhibited somewhat shorter t_{1/2}, but significantly lower CL and much lower V_{d} values than z-DTP2. As a result of these properties, the predicted doses of DTP3 and z-DTP2 required to achieve the therapeutic plasma concentration of 1 μM at the steady state, on the basis of the measured pharmacokinetic values and using the equation described in the Supplemental Experimental Procedures, were 0.81 mg/kg/hr and 3.61 mg/kg/hr, respectively, thus demonstrating the superior pharmacokinetic profile of DTP3 as compared with z-DTP2. Values denote means±SD (n=3) or SEM (n=5), as indicated. MW, molecular weight.

FIG. 16 shows the volumes of subcutaneous U266 myeloma xenografts in mice treated by continual infusion with DTP3 at the dose of 14.5 mg/kg/day or PBS for the times shown. Values denote means±SEM (n=16). ***, p<0.001.

FIG. 17 shows images of representative myeloma-bearing mice (top) and isolated tumors (bottom) from the experiment shown in FIG. 16 at day 28.

FIG. 18 shows percentage survival of mice bearing medullary KMS-12 multiple myeloma xenografts and treated intermittently by infusion with DTP3 at the dose of 29.0 mg/kg/day or PBS (left; n=8, each group) for 8 weeks.

FIG. 19 shows the median OS of each animal cohort from the experiment shown in FIG. 18. *** p<0.0001.

FIG. 20 shows a schematic representation of the Gadd45β/MKK7-targeting strategy compared with conventional therapeutic strategies also aimed at inhibiting the NF-κB pathway in cancer.

FIG. 21 shows the results of qRT-PCR assays showing the relative mRNA levels of (A) GADD45β and (B) MKK7 in a panel of genetically heterogeneous DLBCL cell lines of both the ABC and GCB subtypes. PMBC and HEK-293T cells and a multiple myeloma cell line are used as negative and positive controls, respectively.

FIG. 22 shows overall survival in newly diagnosed, previously untreated patients with DLBCL (A) or HL (B).

FIG. 23 shows propidium iodide (PI) nuclear staining assays showing apoptotic cells (i.e. cells with sub-G₁ DNA content) in DLBCL cell lines after treatment with 10 μM of DTP3 or scrambled (SCR) control D-tripeptide for 6 days. FIG. 23A shows the data from cell lines insensitive to DTP3 and FIG. 23B shows the data from cell lines sensitive to DTP3.

FIG. 24 shows correlation plots of the relative mRNA levels of GADD45β (A) and MKK7 (B), as determined by qRT-PCR, and the percentage of apoptotic cells after treatment with DTP3 (10 μM), as determined by PI nuclear staining assays. r_{S}, Spearman correlation coefficient.

FIG. 25A shows the relative GADD45β mRNA expression levels (qRT-PCR assays; top) and the logarithm to base 10 (log₁₀) of the IC₅₀ values of DTP3 at 144 hrs ([³H]thymidine incorporation assays; bottom) in a panel of tumour cell lines of different tissues of origin. Values denote means±SD (n=3). FIG. 25B shows a Spearman correlation between the two axes of FIG. 25A.

FIG. 26 shows the mutation status of the oncogenic alterations of upstream regulators of the NF-κB pathway most frequently found in primary human DLBCL in the ABC-DLBCL and GCB-DLBCL cell lines that were used to generate the data shown in this application

### DETAILED DESCRIPTION OF THE INVENTION

### Note on Nomenclature Used Herein

In various parts of this specification, compounds are referred to by a signifying code such as LTP, DTP, LNC, DTP1 etc. Codes containing “NC” describe compounds which are negative controls not encompassed within the scope of the invention. Codes containing “TP” (which is an abbreviation of for tetra or tri-peptide/peptoids, although it should be noted that some of the compounds are based on di-peptide/peptoid motifs) are within the scope of the invention. The “L” or “D” prefix denotes residues in the L or D optical configuration. A numeric suffix denotes a specific numbered compound detailed elsewhere. The prefix “Z” as in “Z-DTP” denotes a benzyloxycarbonyl N-terminal group. The “m” prefix as in “mDTP” denotes any modification of a DTP aimed at improving cellular uptake, cellular activity, and/or PK profile, such as the removal of the N and/or C terminus (e.g. as in mDTP1), the removal of the Z group and of the Arg or Glu residues of Z-DTP2 as in mDTP2 and mDTP3, respectively.

### Gadd45β/MKK7 Inhibitors

As discussed above, the present invention is based on the discovery that Gadd45β/MKK7 inhibitors are surprisingly effective therapeutic agents useful for the treatment of haematological malignancies such as multiple myeloma and diffuse large B-cell lymphoma, and in particular resistant forms of those haematological malignancies.

The concept of targeting Gadd45β/MKK7 as a means of providing a therapy for haematological malignancies is based on the understanding that NF-κB-JNK crosstalk also controls survival versus programmed cell death of cells including cancer cells which would otherwise have died. Gadd45β inhibits apoptosis by suppressing JNK signalling. It mediates this function by binding to the JNK kinase, MKK7, and blocking its enzymatic activity by engaging the kinase catalytic pocket (Papa et al, 2004; Papa et al, 2007). Accordingly, a Gadd45β/MKK7 inhibitor is a substance which binds to MKK7 and which inhibits the ability of Gadd45β to bind to MKK7 and thereby to suppress MKK7/JNK-mediated programmed cell death.

In one embodiment, the Gadd45β/MKK7 inhibitor is a peptide. In one embodiment, the Gadd45β/MKK7 inhibitor is a small molecule (e.g. a compound having a molecular weight of less than 1000 Da, preferably less than 750 Da, more preferably less than 600 Da, still more preferably less than 500 Da). A number of specific Gadd45β/MKK7 inhibitors are discussed in PCT/GB2010/001970 and the present invention encompasses the use of those inhibitors, which are also described below.

Gadd45β/MKK7 inhibitors may for example be compounds of formula I:

X₁-A-X₂ I:

wherein,
A is A″″,
   or A″-[M-A′-]ₙ′″;
A″″ is A″,
   A′″,
   or Z₁—Y₂-Y₃—Z₄, wherein Y₂-Y₃ is an oligopeptide moiety or an oligopeptoid moiety having the residues Y₂-Y₃ and Z₁ is attached to the N-terminal nitrogen of Y₂-Y₃ and Z₄ is attached to the C-terminal carbon of Y₂-Y₃;
A″ is A′,
   or Y₁-Y₂—Y₃—Z₄, wherein Y₁-Y₂—Y₃ is an oligopeptoid moiety or an oligopeptoid moiety comprising the residues: Y₁-Y₂—Y₃ and Z₄ is attached to the C-terminal carbon of Y₁-Y₂—Y₃;
A′″ is A′,
   or Z₁—Y₂-Y₃—Y₄, wherein Y₂-Y₃—Y₄ is an oligopeptoid moiety or an oligopeptoid moiety comprising the residues Y₂-Y₃—Y₄ and Z₁ is attached to the N-terminal nitrogen of Y₂-Y₃—Y₄;
each occurrence of A′ is independently an oligopeptide moiety or an oligopeptoid moiety comprising the residues Y₁-Y₂—Y₃—Y₄;
n is an integer from 0 to 18
Y₁ and Y₄ are independently amino acid residues or residues of amino acid derivatives having aromatic side chains
Y₂ is an amino acid residue or a residue of an amino acid derivative or is absent;
Y₃ is an amino acid residue or a residue of an amino acid derivative or is absent;
   Z₁ is a group of formula II:

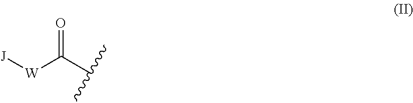

which is linked to the N-terminal nitrogen of Y₂,
W is absent, or an oxygen, or a nitrogen, or an alkylene group of from one to three carbons,
which alkylene group of from one to three carbons is optionally substituted by at least one substituent selected from alkyl of from one to four carbons, or 5-10 membered carbocyclic or heterocyclic aromatic group;
J is a 5-10 membered carbocyclic or heterocyclic aromatic group,
which aromatic group is optionally substituted by at least one substituent selected from hydroxyl, halogen, alkyl of from one to four carbons, or alkoxy of from one to four carbon atoms;
Z₄ represents a group of formula III:

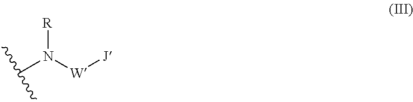

which is linked to the C-terminal carbon of Y₃,
R is hydrogen or alkyl of from one to four carbons;
W′ is absent or an alkylene group of from one to three carbons,
which alkylene group of from one to three carbons is optionally substituted by at least one substituent selected from alkyl of from one to four carbons, or 5-10 membered carbocyclic or heterocyclic aromatic group;
J′ is a 3-10 membered aliphatic carbocyclic group or a 5-10 membered carbocyclic or heterocyclic aromatic group,
which aliphatic or aromatic group is optionally substituted by at least one substituent selected from hydroxyl, halogen, alkyl of from one to four carbons, or alkoxy of from one to four carbon atoms;
M is a peptide bond between preceding oligopeptide or oligopeptoid moiety (A′, A″ or A′″) and following oligopeptide or oligopeptide moiety (A′, A″ or A′″) or a linker moiety attached via an amide bond, an ester bond, an ether bond, or a thioether bond to the terminal carboxylic group of preceding oligopeptide or oligopeptoid moiety (A′, A″ or A′″) and via an amide bond, an ester bond, an ether bond, or a thioether bond to the terminal amino group of following oligopeptoid moiety (A′, A″ or A′);
X₁ is absent, or is a moiety added to the amino terminal of A in order to block the free amino group;
X₂ is absent or is a moiety added to the carboxyl terminal of A in order to block the free carboxyl group;
with the proviso that X₁ is absent if A comprises Z₁ and X₂ is absent if A comprises Z₄;
or derivatives thereof, said derivatives being selected from the group consisting of:
a) oligomers or multimers of molecules of the compound of formula I, said oligomers and multimers comprising two or more molecules of the compound of formula I each linked to a common scaffold moiety via an amide bond formed between an amino or carboxylic acid group present in molecules of the compound of formula I and an opposite amino or carboxylic acid group on a scaffold moiety said scaffold moiety participating in at least 2 amide bonds,
b) derivatives comprising a molecule of the compound of formula I or an oligomer or multimer thereof as defined above in part a) conjugated via an amide bond, an ester bond, an ether bond or a thioether bond to:
   PEG,
   PEG-based compounds,
   cell-penetrating peptides,
   fluorescent dyes,
   biotin or other tag moiety,
   fatty acids,
   nanoparticles of discrete size
   or chelating ligands complexed with metallic or
   radioactive ions.
c) derivatives comprising a molecule of the compound of formula I or an oligomer or multimer thereof as defined in part a) which has been modified by amidation, glycosylation, carbamylation, acylation, sulfation, phosphorylation, cyclization, lipidisation, pegylation or linkage to a peptide or peptoid fusion partner to make a fusion peptide or fusion peptoid.
   and
d) salts and solvates of a molecule of the compound of formula I or of a derivative thereof as defined in part a) or b) above.

According to certain embodiments:
Y₁ is D-tryptophan;
   L-tryptophan;
   D-tyrosine;
   L-tyrosine;
   D-3,3-diphenyl-alanine;
   L-3,3-diphenyl-alanine;
   D-H-3-(4-pyridyl) alanine;
   L-H-3-(4-pyridyl) alanine;
   D-H-3-(3-pyridyl) alanine;
   L-H-3-(3-pyridyl) alanine;
   D-H-3-(2-pyridyl) alanine;
   L-H-3-(2-pyridyl) alanine;
   D-2-amino-4-phenyl-butyric acid;
   L-2-amino-4-phenyl-butyric acid;
   D-H-4-hydroxy-phenyl-glycine;
   L-H-4-hydroxy-phenyl-glycine;
   D-3-(2-furyl)-alanine;
   L-3-(2-furyl)-alanine;
   L-homoPhenylalanine;
   D-homoPhenylalanine;
   D-3-(4-quinolyl)-alanine;
   L-3-(4-quinolyl)-alanine;
   D-napthyl-alanine;
   L-napthyl-alanine;
   p-hydroxy-Benzoic acid;
   p-hydroxy-phenyl-acetic-acid;
   3-(p-hydroxy-phenyl)-propionic-acid;
   Or N-methyl-derivatives in L- or D-configuration of any of the above.

Alternatively Y₁ may be:
D-phenylalanine;
L-phenylalanine;
D-tryptophan;
L-tryptophan;
D-tyrosine;
L-tyrosine;
D-3,3-diphenyl-alanine;
L-3,3-diphenyl-alanine;
D-H-3-(4-pyridyl) alanine;
L-H-3-(4-pyridyl) alanine;
D-H-3-(3-pyridyl) alanine;
L-H-3-(3-pyridyl) alanine;
D-H-3-(2-pyridyl) alanine;
L-H-3-(2-pyridyl) alanine;
D-2-amino-4-phenyl-butyric acid;
L-2-amino-4-phenyl-butyric acid;
D-phenyl-glycine;
L-phenyl-glycine;
D-H-4-hydroxy-phenyl-glycine;
L-H-4-hydroxy-phenyl-glycine;
D-3-(2-furyl)-alanine;
L-3-(2-furyl)-alanine;
L-Cyclohexylalanine;
D-Cyclohexylalanine;
L-homoPhenylalanine;
D-homoPhenylalanine;
D-3-(4-quinolyl)-alanine;
L-3-(4-quinolyl)-alanine;
D-napthyl-alanine;
or L-napthyl-alanine.

According to certain embodiments:
Y₂ is absent;
   D-glutamic acid;
   L-glutamic acid;
   D-aspartic acid;
   L-aspartic acid;
   L-Leucine;
   D-Leucine;
   L-Glutamine;
   D-Glutamine;
   L-Methionine;
   D-Methionine;
   D-2-amino-heptanedioic acid;
   L-2-amino-heptanedioic acid;
   a methyl or ethyl ester of any thereof;
   L-homoserine;
   D-homoserine;
   or N-methyl-derivatives in L- or D-configuration of any of the above.

Alternatively Y₂ may be:
D-glutamic acid;
L-glutamic acid;
D-aspartic acid;
L-aspartic acid;
D-2-amino-heptanedioic acid;
L-2-amino-heptanedioic acid;
a methyl or ethyl ester of any thereof;
L-homoserine;
or D-homoserine.

According to certain embodiments:
Y₃ is D-arginine;
   L-arginine;
   L-Proline;
   D-Proline;
   D-histidine;
   L-histidine;
   D-lysine;
   D-α,β-diaminopropionic acid (D-Dap);
   L-α,β-diaminopropionic acid (L-Dap);
   L-α,δ-diaminobutyric acid (L-Dab);
   L-α,δ-diaminobutyric acid (L-Dab);
   L-ornithine;
   D-ornithine;
   L-lysine;
   or N-methyl-derivatives in L- or D-configuration of any of the above.

Alternatively Y₃ may be:
D-arginine;
L-arginine;
D-histidine;
L-histidine;
D-lysine;
D-α,β-diaminopropionic acid (D-Dap);
L-α,β-diaminopropionic acid (L-Dap);
L-α,δ-diaminobutyric acid (L-Dab);
L-α,δ-diaminobutyric acid (L-Dab);
L-ornithine;
D-ornithine;
or L-lysine.

According to certain embodiments:
Y₄ iS
   D-phenylalanine;
   L-phenylalanine;
   D-tryptophan;
   L-tryptophan;
   D-tyrosine;
   L-tyrosine;
   D-3,3-diphenyl-alanine;
   L-3,3-diphenyl-alanine;
   D-H-3-(4-pyridyl) alanine;
   L-H-3-(4-pyridyl) alanine;
   D-H-3-(3-pyridyl) alanine;
   L-H-3-(3-pyridyl) alanine;
   D-H-3-(2-pyridyl) alanine;
   L-H-3-(2-pyridyl) alanine;
   D-2-amino-4-phenyl-butyric acid;
   L-2-amino-4-phenyl-butyric acid;
   D-phenyl-glycine;
   L-phenyl-glycine;
   D-H-4-hydroxy-phenyl-glycine;
   L-H-4-hydroxy-phenyl-glycine;
   D-3-(2-furyl)-alanine;
   L-3-(2-furyl)-alanine;
   L-homoPhenylalanine;
   D-homoPhenylalanine;
   D-3-(4-quinolyl)-alanine;
   L-3-(4-quinolyl)-alanine;
   D-napthyl-alanine;
   L-napthyl-alanine;
   their N-methyl-derivatives in L- or
   D-configuration;
   aniline;
   benzylamine;
   or 2-phenyl-ethyl-amine.

Alternatively Y₄ may be:
D-phenylalanine;
L-phenylalanine;
D-tryptophan;
L-tryptophan;
D-tyrosine;
L-tyrosine;
D-3,3-diphenyl-alanine;
L-3,3-diphenyl-alanine;
D-H-3-(4-pyridyl) alanine;
L-H-3-(4-pyridyl) alanine;
D-H-3-(3-pyridyl) alanine;
L-H-3-(3-pyridyl) alanine;
D-H-3-(2-pyridyl) alanine;
L-H-3-(2-pyridyl) alanine;
D-2-amino-4-phenyl-butyric acid;
L-2-amino-4-phenyl-butyric acid;
D-phenyl-glycine;
L-phenyl-glycine;
D-H-4-hydroxy-phenyl-glycine;
L-H-4-hydroxy-phenyl-glycine;
D-3-(2-furyl)-alanine;
L-3-(2-furyl)-alanine;
L-Cyclohexylalanine;
D-Cyclohexylalanine;
L-homoPhenylalanine;
D-homoPhenylalanine;
D-3-(4-quinolyl)-alanine;
L-3-(4-quinolyl)-alanine;
D-napthyl-alanine;
or L-napthyl-alanine.
According to certain preferred embodiments Y₁, Y₂, Y₃ and Y₄ are all as described above. According to certain embodiments Y₁, Y₂, Y₃ and Y₄ are all described above with the proviso that Y₂ is
   D-glutamic acid;
   L-glutamic acid;
   D-aspartic acid;
   L-aspartic acid;
   D-2-amino-heptanedioic acid;
   L-2-amino-heptanedioic acid;
   a methyl or ethyl ester of any thereof;
   L-homoserine;
   L-Leucine;
   D-Leucine;
   L-Glutamine;
   D-Glutamine;
   L-Methionine;
   D-Methionine;
   D-homoserine;
   or N-methyl-derivatives in L- or D-configuration of any of the above;
and Y₃ is
   D-arginine;
   L-arginine;
   D-histidine;
   L-histidine;
   D-lysine;
   L-lysine;
   L-Proline;
   D-Proline;
   D-α,β-diaminopropionic acid (D-Dap);
   L-α,β-diaminopropionic acid (L-Dap);
   D-α,δ-diaminobutyric acid (D-Dab);
   L-α,δ-diaminobutyric acid (L-Dab);
   D-ornithine;
   L-ornithine;
   or N-methyl-derivatives in L- or D-configuration of any of the above.

According to certain embodiments Y₁ and Y₂ are both as described above but one or both of Y₂ and Y₃ are absent. According to certain embodiments M is a peptide bond.

According to certain embodiments X₁ is a hydrogen or X₁ is one of the following groups added to the amino terminal of the oligopeptide sequence so as to form an amide bond:
Acetyl;
Benzyloxycarbonyl;
2-chloro-benzyloxycarbonyl;
3-methoxy-4-hydroxy-benzoyl;
3-hydroxy-4-methoxy-benzoyl;
Benzoyl;
or fluorenylmethoxycarbonyl;

X₂ is a hydroxyl group or is one of the following groups added to the carbonyl acid terminal of the oligopeptide sequence so as to form an amide bond:
amine;
D-phenylalanine;
L-phenylalanine;
D-tryptophan;
L-tryptophan;
D-tyrosine;
L-tyrosine;
D-3,3-diphenyl-alanine;
L-3,3-diphenyl-alanine;
D-H-3-(4-pyridyl)-alanine;
L-H-3-(4-pyridyl)-alanine;
D-H-3-(3-pyridyl)-alanine;
L-H-3-(3-pyridyl)-alanine;
D-H-3-(2-pyridyl)-alanine;
L-H-3-(2-pyridyl)-alanine;
D-2-amino-4-phenyl-butyric acid;
L-2-amino-4-phenyl-butyric acid;
D-phenyl-glycine;
L-phenyl-glycine;
D-H-4-hydroxy-phenyl-glycine;
L-H-4-hydroxy-phenyl-glycine;
D-3-(2-furyl)-alanine;
L-3-(2-furyl)-alanine;
L-Cyclohexylalanine;
D-Cyclohexylalanine;
L-homoPhenylalanine;
D-homoPhenylalanine;
D-3-(4-quinolyl)-alanine;
L-3-(4-quinolyl)-alanine;
D-napthyl-alanine;
L-napthyl-alanine;
or N-methyl-derivatives in L- or D-configuration of any of the above.

According to certain embodiments:
Z₁ is 4-hydroxy-benzoyl;
   (4-hydroxy-phenyl)-acetyl;
   3-(4-hydroxy-phenyl)-propionyl;
   Benzoyl;
   Benzyloxycarbonyl;
   2-phenyl-acetyl;
   3-phenyl-propionyl;
   3,3-diphenyl-propionyl;
   3-(1H-Indol-3yl)-propionyl;
   (1H-Indol-3-yl)-acetyl;
   Furan-2-yl-acetyl;
   Furan-3-yl-acetyl;
   3-pyridin-4-yl-propionyl;
   3-pyridin-3-yl-propionyl;
   3-pyridin-2-yl-propionyl;
   3-pyrimidin-4-yl-propionyl;
   3-pyridazin-4-yl-propionyl;
   3-[1,3,5]Triazin-2-yl-propionyl;
   2-pyridin-4-yl-acetyl;
   2-pyridin-3-yl-acetyl;
   2-pyridin-2-yl-acetyl;
   2-pyrimidin-4-yl-acetyl;
   2-pyridazin-4-yl-acetyl;
   2-[1,3,5]Triazin-2-yl-acetyl;
   Naphthalen-1-yl-acetyl;
   Naphthalen-2-yl-acetyl;
   2-Naphthalen-1-yl-propionyl;
   or 2-Naphthalen-2-yl-propionyl;
Y₂ is D-glutamic acid;
   L-glutamic acid;
   D-aspartic acid;
   L-aspartic acid;
   L-Leucine;
   D-Leucine;
   L-Glutamine;
   D-Glutamine;
   L-Methionine;
   D-Methionine;
   D-2-amino-heptanedioic acid;
   L-2-amino-heptanedioic acid;
   a methyl or ethyl ester of any thereof;
   L-homoserine;
   D-homoserine;
   or N-methyl-derivatives in L- or D-configuration of any of the above;
Y₃ is D-arginine;
   L-arginine;
   D-histidine;
   L-histidine;
   L-proline;
   D-proline;
   D-lysine;
   L-lysine;
   D-α,β-diaminopropionic acid (D-Dap);
   L-α,β-diaminopropionic acid (L-Dap);
   D-α,δ-diaminobutyric acid (D-Dab);
   L-α,δ-diaminobutyric acid (L-Dab);
   D-ornithine;
   L-ornithine;
   or N-methyl-derivatives in L- or D-configuration of any above;
Z₄ is Phenylamine;
   Benzylamine;
   Phenethylamine;
   Cyclohexyl-amine;
   2-cyclohexyl-ethylamine;
   3-cyclohexyl-propylamine;
   4-(2-amino-ethyl)-phenol;
   4-amino-phenol;
   4-aminomethyl-phenol;
   1H-Indol-3-yl-amine;
   2-(1H-Indol-3-yl)-ethylamine;
   C-(1H-Indol-3-yl)-methylamine;
   2,2-diphenyl-ethylamine;
   2,2-dipyridin-4-yl-ethylamine;
   2-pyridin-4-yl-ethylamine;
   2-pyridin-3-yl-ethylamine;
   2-pyridin-2-yl-ethylamine;
   2-pyrimidin-4-yl-ethylamine;
   2-[1,3,5]Triazin-2-yl-ethylamine;
   C-furan-2-yl-methylamine;
   C-furan-3-yl-methylamine;
   or C-Naphthalen-2-yl-methylamine.

According to the convention all peptides and peptoids and regions thereof are described from the N terminus to the C terminus.

n may be 0, 1, 2, 3, 4, 5, 6, 7, 8, 9, 10, 11, 12, 13, 14, 15, 16, 17 or 18. According to certain preferred embodiments n=0.

According to certain preferred embodiments A is A′. In such embodiments the compound is therefore essentially a tetrapeptide, a tripeptide, or a dipeptide (or a corresponding peptoid) with optional blocking groups X₁ and X₂ at one or more of the termini.

### Oligopeptides

Oligopeptides are short polymers formed by the condensation of α-amino acids (referred to herein as simply “amino acids”). The link between one amino acid residue and the next is known as a peptide bond or an amide bond.

### Amino-Acids

As used herein the term “amino acid” includes the 20 standard amino acids (Isoleucine, Alanine, Leucine, Asparagine, Lysine, Aspartic Acid, Methionine, Cysteine, Phenylalanine, Glutamic Acid, Threonine, Glutamine, Tryptophan, Glycine, Valine, Proline, Serine, Tyrosine, Arginine and Histidine) in both their D and L optical configurations. It also includes synthetic α-amino acids in both D and L forms. According to certain embodiments the D configuration is preferred.

### Amino Acid Derivatives

As used herein this term includes N-substituted glycines which differ from α-amino acids in that their side chains are appended to nitrogen atoms along the molecule's backbone, rather than to the α-carbons (as they are in amino acids). Also included in the term are methyl and ethyl esters of α-amino acids, β-amino acids and N-methylated α-amino acids.

### Oligopeptoids

Strictly speaking, the term “oligopeptide” relates to oligomers of α-amino acids only. An analogous oligomer incorporating (at all or some residue positions) an amino acid derivate (for example an N-substituted glycine) is known as an oligopeptoid.

### Derivatives

Derivatives of the Gadd45β/MKK7 inhibitors exemplified here may be functional derivatives. The term “functional derivative” is used herein to denote a chemical derivative of a compound of formula (I) having the same physiological function (as the corresponding unmodified compounds of formula (I) or alternatively having the same in vitro function in a functional assay (for example, in one of the assays described in one of the examples disclosed herein).

Derivatives of the compounds may comprise the structure of formula (I) modified by well-known processes including amidation, glycosylation, carbamylation, acylation, for example acetylation, sulfation, phosphorylation, cyclization, lipidization and pegylation. The structure of formula (I) may be modified at random positions within the molecule, or at predetermined positions within the molecule and may include one, two, three or more attached chemical moieties. Derivatives include compounds in which the N-terminal NH₂ group is replaced with another group, for example a methoxy group.

A Gadd45β/MKK7 inhibitor may be a fusion protein, whereby the structure of formula (I) is fused to another protein or polypeptide (the fusion partner) using methods known in the art. Any suitable peptide or protein can be used as the fusion partner (e.g., serum albumin, carbonic anhydrase, glutathione-S-transferase or thioredoxin, etc.). Preferred fusion partners will not have an adverse biological activity in vivo. Such fusion proteins may be made by linking the carboxy-terminus of the fusion partner to the amino-terminus of the structure of formula (I) or vice versa. Optionally, a cleavable linker may be used to link the structure of formula (I) to the fusion partner. A resulting cleavable fusion protein may be cleaved in vivo such that an active form of the inhibitor is released. Examples of such cleavable linkers include, but are not limited to, the linkers D-D-D-D-Y [SEQ ID NO.: 99], G-P-R, A-G-G and H—P-F-H-L [SEQ ID NO.: 100], which can be cleaved by enterokinase, thrombin, ubiquitin cleaving enzyme and renin, respectively. See, e.g., U.S. Pat. No. 6,410,707.

Examples of derivatives include esters, amides, and carbamates; preferably esters and amides. Pharmaceutically acceptable esters and amides of the compounds of formula (I) may comprise a C₁₋₂₀ alkyl-, C₂₋₂₀ alkenyl-, C₅₋₁₀ aryl-, C₅₋₁₀ or C₁₋₂₀ alkyl-, or amino acid-ester or -amide attached at an appropriate site, for example at an acid group. Examples of suitable moieties are hydrophobic substituents with 4 to 26 carbon atoms, preferably 5 to 19 carbon atoms. Suitable lipid groups include, but are not limited to, the following: lauroyl (Ci₂H₂₃), palmityl (Ci₅H₃₁), oleyl (C₁₅H₂₉), stearyl (C₁₇H₃₅), cholate; and deoxycholate.

Methods for lipidization of sulfhydryl-containing compounds with fatty acid derivatives are disclosed in U.S. Pat. No. 5,936,092; U.S. Pat. No. 6,093,692; and U.S. Pat. No. 6,225,445. Fatty acid derivatives of in inhibitor comprising an inhibitor linked to fatty acid via a disulfide linkage may be used for delivery of an inhibitor to neuronal cells and tissues. Lipidisation markedly increases the absorption of the compounds relative to the rate of absorption of the corresponding unlipidised compounds, as well as prolonging blood and tissue retention of the compounds. Moreover, the disulfide linkage in lipidised derivative is relatively labile in the cells and thus facilitates intracellular release of the molecule from the fatty acid moieties. Suitable lipid-containing moieties are hydrophobic substituents with 4 to 26 carbon atoms, preferably 5 to 19 carbon atoms. Suitable lipid groups include, but are not limited to, the following: palmityl (C₁₅H₃₁), oleyl (C₁₅H₂₉), stearyl (C₁₇H₃₅), cholate; and deoxycholate.

Cyclization methods include cyclization through the formation of a disulfide bridge and head-to-tail cyclization using a cyclization resin. Cyclized peptides may have enhanced stability, including increased resistance to enzymatic degradation, as a result of their conformational constraints. Cyclization may in particular be expedient where the uncyclized peptide includes an N-terminal cysteine group. Suitable cyclized peptides include monomeric and dimeric head-to-tail cyclized structures. Cyclized peptides may include one or more additional residues, especially an additional cysteine incorporated for the purpose of formation of a disulfide bond or a side chain incorporated for the purpose of resin-based cyclization.

A compound may be a pegylated structure of formula (I). Pegylated inhibitor compounds may provide additional advantages such as increased solubility, stability and circulating time of the polypeptide, or decreased immunogenicity (see U.S. Pat. No. 4,179,337).

Chemical moieties for derivitization of a compound may also be selected from water soluble polymers such as polyethylene glycol, ethylene glycol/propylene glycol copolymers, carboxymethylcellulose, dextran, polyvinyl alcohol and the like. A polymer moiety for derivatisation of an inhibitor may be of any molecular weight, and may be branched or unbranched. Polymers of other molecular weights may be used, depending on the desired therapeutic profile, for example the duration of sustained release desired, the effects, if any on biological activity, the ease in handling, the degree or lack of antigenicity and other known effects of the polyethylene glycol to a therapeutic protein or analog. For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 Da.

Salts and solvates of compounds that are suitable for use in a medicament are those wherein a counterion or associated solvent is pharmaceutically acceptable. However, salts and solvates having non-pharmaceutically acceptable counterions or associated solvents may also be used, for example, for use as intermediates in the preparation of the compounds of formula (I) and their pharmaceutically acceptable salts or solvates.

Suitable salts include those formed with organic or inorganic acids or bases. Pharmaceutically acceptable acid addition salts include those formed with hydrochloric, hydrobromic, sulfuric, nitric, citric, tartaric, acetic, phosphoric, lactic, pyruvic, acetic, trifluoroacetic, succinic, perchloric, fumaric, maleic, glycolic, lactic, salicylic, oxaloacetic, methanesulfonic, ethanesulfonic, p-toluenesulfonic, formic, benzoic, malonic, naphthalene-2-sulfonic, benzenesulfonic and isethionic acids. Other acids such as oxalic, while not in themselves pharmaceutically acceptable, may be useful as intermediates in obtaining the inhibitor compounds and their pharmaceutical acceptable salts. Pharmaceutically acceptable salts with bases include ammonium salts, alkali metal salts, for example potassium and sodium salts, alkaline earth metal salts, for example calcium and magnesium salts, and salts with organic bases, for example dicyclohexylamine and N-methyl-D-glucomine.

Those skilled in the art of organic chemistry will appreciate that many organic compounds can form complexes with solvents in which they are reacted or from which they are precipitated or crystallized. Such complexes are known as “solvates”. For example, a complex with water is known as a “hydrate”.

According to certain preferred embodiments, the Gadd45β/MKK7 inhibitor has a half-life in the human circulation of at least 0.1, 0.2, 0.5, 1, 2, 3, 4, 5, 6, 7, 8, 9, 10, 11 or most preferably at least 12 hours.

Preferably, the Gadd45β/MKK7 inhibitor retains at least 20, 30, 40, 50, 60, 70, 80, 90 or most preferably 99% of its capacity to bind to MKK7 as assessed in an in vitro binding assay, or at least 20, 30, 40, 50, 60, 70, 80, 90 or most preferably 99% of its capacity to block the Gadd45β interaction with MKK7 as assessed in an in vitro competitive binding assay following incubation in normal human serum for at 24 hours at 37 degrees Celsius.

Alternatively or additionally, the Gadd45β/MKK7 inhibitor has at least the ability to inhibit at least 20, 30, 40, 50, 60, 70, 80, 90 or most preferably 99% of the MKK7 interactions with Gadd45β under the assay conditions described in the examples below and/or under the assay conditions described in WO2011/048390.

According to certain preferred embodiments the oligopeptide core moiety of the compound, identified as A in Formula I, has an amino acid sequence selected from the group consisting of:

[SEQ ID NO.: 1]
(L-Tyr)-(L-Asp)-(L-His)-(L-Phe);
[SEQ ID NO.: 2]
(L-Tyr)-(L-Glu)-(L-Arg)-(L-Phe);
[SEQ ID NO.: 3]
(L-Tyr)-(L-Glu)-(L-His)-(L-Phe);
[SEQ ID NO.: 4]
(L-Trp)-(L-Asp)-(L-His)-(L-Phe);
[SEQ ID NO.: 5]
(L-Trp)-(L-Glu)-(L-His)-(L-Phe);
[SEQ ID NO.: 6]
(L-Tyr)-(L-Asp)-(L-Arg)-(L-Phe);
[SEQ ID NO.: 7]
(L-Tyr)-(L-Asp)-(L-Lys)-(L-Phe);
[SEQ ID NO.: 8]
(L-Tyr)-(L-Glu)-(L-Lys)-(L-Phe);
[SEQ ID NO.: 9]
(L-Trp)-(L-Glu)-(L-Lys)-(L-Phe);
[SEQ ID NO.: 10]
(L-Trp)-(L-Glu)-(L-Arg)-(L-Phe);
[SEQ ID NO.: 11]
(L-Trp)-(L-Asp)-(L-Lys)-(L-Phe);
[SEQ ID NO.: 12]
(L-Trp)-(L-Asp)-(L-Arg)-(L-Phe);
[SEQ ID NO.: 13]
(L-Tyr)-(L-Asp)-(L-His)-(L-Trp);
[SEQ ID NO.: 14]
(L-Tyr)-(L-Glu)-(L-His)-(L-Trp);
[SEQ ID NO.: 15]
(L-Trp)-(L-Asp)-(L-His)-(L-Trp);
[SEQ ID NO.: 16]
(L-Trp)-(L-Glu)-(L-His)-(L-Trp);
[SEQ ID NO.: 17]
(L-Tyr)-(L-Asp)-(L-Arg)-(L-Trp);
[SEQ ID NO.: 18]
(L-Tyr)-(L-Asp)-(L-Lys)-(L-Trp);
[SEQ ID NO.: 19]
(L-Tyr)-(L-Glu)-(L-Lys)-(L-Trp);
[SEQ ID NO.: 20]
(L-Tyr)-(L-Glu)-(L-Arg)-(L-Trp);
[SEQ ID NO.: 21]
(L-Trp)-(L-Glu)-(L-Lys)-(L-Trp);
[SEQ ID NO.: 22]
(L-Trp)-(L-Glu)-(L-Arg)-(L-Trp);
[SEQ ID NO.: 23]
(L-Trp)-(L-Asp)-(L-Lys)-(L-Trp);
[SEQ ID NO.: 24]
(L-Trp)-(L-Asp)-(L-Arg)-(L-Trp);
[SEQ ID NO.: 25]
(L-Tyr)-(L-Asp)-(L-His)-(L-Tyr);
[SEQ ID NO.: 26]
(D-Tyr)-(D-Glu)-(D-Arg)-(D-Phe);
[SEQ ID NO.: 27]
(D-Tyr)-(D-Asp)-(D-His)-(D-Phe);
[SEQ ID NO.: 28]
(D-Trp)-(D-Glu)-(D-Arg)-(D-Phe);
[SEQ ID NO.: 29]
(D-Trp)-(D-Asp)-(D-His)-(D-Phe);
[SEQ ID NO.: 30]
(D-Tyr)-(D-Asp)-(D-Arg)-(D-Phe);
[SEQ ID NO.: 31]
(D-Tyr)-(D-Asp)-(D-His)-(D-Tyr);
[SEQ ID NO.: 32]
(D-Tyr)-(D-Glu)-(D-Arg)-(D-Tyr);
[SEQ ID NO.: 33]
(D-Trp)-(D-Asp)-(D-His)-(D-Tyr);
[SEQ ID NO.: 34]
(D-Trp)-(D-Glu)-(D-Arg)-(D-Tyr);
[SEQ ID NO.: 35]
(D-Tyr)-(D-Asp)-(D-Lys)-(D-Phe);
[SEQ ID NO.: 36]
(D-Tyr)-(D-Glu)-(D-His)-(D-Phe);
[SEQ ID NO.: 37]
(D-Tyr)-(D-Asp)-(D-Lys)-(D-Phe);
[SEQ ID NO.: 38]
(D-Trp)-(D-Glu)-(D-His)-(D-Phe);
[SEQ ID NO.: 39]
(D-Tyr)-(D-Glu)-(D-Lys)-(D-Phe);
[SEQ ID NO.: 40]
(D-Trp)-(D-Glu)-(D-Lys)-(D-Phe);
[SEQ ID NO.: 41]
(D-Trp)-(D-Asp)-(D-Lys)-(D-Phe);
[SEQ ID NO.: 42]
(D-Tyr)-(D-Asp)-(D-His)-(D-Trp);
[SEQ ID NO.: 43]
(D-Tyr)-(D-Glu)-(D-His)-(D-Trp);
[SEQ ID NO.: 44]
(D-Trp)-(D-Asp)-(D-His)-(D-Trp);
[SEQ ID NO.: 45]
(D-Trp)-(D-Glu)-(D-His)-(D-Trp);
[SEQ ID NO.: 46]
(D-Tyr)-(D-Asp)-(D-Arg)-(D-Trp);
[SEQ ID NO.: 47]
(D-Tyr)-(D-Asp)-(D-Lys)-(D-Trp);
[SEQ ID NO.: 48]
(D-Tyr)-(D-Glu)-(D-Lys)-(D-Trp);
[SEQ ID NO.: 49]
(D-Tyr)-(D-Glu)-(D-Arg)-(D-Trp);
[SEQ ID NO.: 50]
(D-Trp)-(D-Glu)-(D-Lys)-(D-Trp);
[SEQ ID NO.: 51]
(D-Trp)-(D-Glu)-(D-Arg)-(D-Trp);
[SEQ ID NO.: 52]
(D-Trp)-(D-Asp)-(D-Lys)-(D-Trp);
[SEQ ID NO.: 53]
(D-Trp)-(D-Gln)-(D-Arg)-(D-Trp);
[SEQ ID NO.: 54]
(D-Trp)-(D-Asn)-(D-Lys)-(D-Trp);
(L-Tyr)-(L-Asp)-(L-Phe);
(D-Tyr)-(D-Asp)-(D-Phe);
(L-Tyr)-(L-Glu)-(L-Phe);
(L-Tyr)-(L-Arg)-(L-Phe);
(D-Tyr)-(D-Arg)-(D-Phe);
(D-Tyr)-(D-Glu)-(D-Phe);
(D-Tyr)-(D-Pro)-(D-Phe);
(D-Tyr)-(D-Leu)-(D-Phe);
(D-Tyr)-(D-Asp)-(D-Tyr);
(D-Tyr)-(D-Glu)-(D-Tyr);
(D-Tyr)-(D-Arg)-(D-Tyr);
(D-Tyr)-(D-Pro)-(D-Tyr);
(D-Tyr)-(D-Leu)-(D-Tyr);
(D-Phe)-(D-Pro)-(D-Phe);
(D-Phe)-(D-Leu)-(D-Phe);
(D-Phe)-(D-Arg)-(D-Tyr);
(D-Phe)-(D-Glu)-(D-Tyr);
(D-Phe)-(D-Asp)-(D-Tyr);
(D-Phe)-(D-Pro)-(D-Tyr);
(D-Phe)-(D-Leu)-(D-Tyr);
(D-Tyr)-(D-Pro)-(D-Trp);
(D-Tyr)-(D-Leu)-(D-Trp);
(D-Tyr)-(D-Asp)-(D-Trp);
(D-Tyr)-(D-Glu)-(D-Trp);
(D-Tyr)-(D-Arg)-(D-Trp);
(D-Tyr)-(D-Pro)-(D-Trp);
(D-Tyr)-(D-Leu)-(D-Trp);
(D-Phe)-(D-Pro)-(D-Trp);
(D-Phe)-(D-Leu)-(D-Trp);
(D-Phe)-(D-Arg)-(D-Trp);
(D-Phe)-(D-Glu)-(D-Trp);
(D-Phe)-(D-Asp)-(D-Trp);
(D-Phe)-(D-Pro)-(D-Trp);
and
(D-Phe)-(D-Leu)-(D-Trp).

In other embodiments the A moiety is selected from the group consisting of:
p-hydroxybenzoic acid-(L-Glu)-(L-Arg)-aniline;
p-hydroxybenzoic acid-(D-Glu)-(L-Arg)-aniline;
p-hydroxybenzoic acid-(L-Glu)-(D-Arg)-aniline;
p-hydroxybenzoic acid-(D-Glu)-(D-Arg)-aniline;
p-hydroxybenzoic acid-(L-Glu)-(L-Arg)-benzylamine;
p-hydroxybenzoic acid-(D-Glu)-(L-Arg)-benzylamine;
p-hydroxybenzoic acid-(L-Glu)-(D-Arg)-benzylamine;
p-hydroxybenzoic acid-(D-Glu)-(D-Arg)-benzylamine;
p-hydroxybenzoic acid-(L-Glu)-(L-Arg)-2-phenyl-ethyl-amine;
p-hydroxybenzoic acid-(D-Glu)-(L-Arg)-2-phenyl-ethyl-amine;
p-hydroxybenzoic acid-(L-Glu)-(D-Arg)-2-phenyl-ethyl-amine;
p-hydroxybenzoic acid-(D-Glu)-(D-Arg)-2-phenyl-ethyl-amine;
2-(4-hydroxy-phenyl) acetic acid-(L-Glu)-(L-Arg)-aniline;
2-(4-hydroxy-phenyl) acetic acid-(D-Glu)-(L-Arg)-aniline;
2-(4-hydroxy-phenyl) acetic acid-(L-Glu)-(D-Arg)-aniline;
2-(4-hydroxy-phenyl) acetic acid-(D-Glu)-(D-Arg)-aniline;
2-(4-hydroxy-phenyl) acetic acid-(L-Glu)-(L-Arg)-benzylamine;
2-(4-hydroxy-phenyl) acetic acid-(D-Glu)-(L-Arg)-benzylamine;
2-(4-hydroxy-phenyl) acetic acid-(L-Glu)-(D-Arg)-benzylamine;
2-(4-hydroxy-phenyl acetic acid-(D-Glu)-(D-Arg)-benzylamine;
2-(4-hydroxy-phenyl) acetic acid-(L-Glu)-(L-Arg)-2-phenyl-ethyl-amine;
2-(4-hydroxy-phenyl) acetic acid-(D-Glu)-(L-Arg)-2-phenyl-ethyl-amine;
2-(4-hydroxy-phenyl) acetic acid-(L-Glu)-(D-Arg)-2-phenyl-ethyl-amine;
2-(4-hydroxy-phenyl) acetic acid-(D-Glu)-(D-Arg)-2-phenyl-ethyl-amine;
3-(4-hydroxy-phenyl) propionic acid-(L-Glu)-(L-Arg)-aniline;
3-(4-hydroxy-phenyl) propionic acid-(D-Glu)-(L-Arg)-aniline;
3-(4-hydroxy-phenyl) propionic acid-(L-Glu)-(D-Arg)-aniline;
3-(4-hydroxy-phenyl) propionic acid-(D-Glu)-(D-Arg)-aniline;
3-(4-hydroxy-phenyl) propionic acid-(L-Glu)-(L-Arg)-benzylamine;
3-(4-hydroxy-phenyl) propionic acid-(D-Glu)-(L-Arg)-benzylamine;
3-(4-hydroxy-phenyl) propionic acid-(L-Glu)-(D-Arg)-benzylamine;
3-(4-hydroxy-phenyl) propionic acid-(D-Glu)-(D-Arg)-benzylamine;
3-(4-hydroxy-phenyl) propionic acid-(L-Glu)-(L-Arg)-2-phenyl-ethyl-amine;
3-(4-hydroxy-phenyl) propionic acid-(D-Glu)-(L-Arg)-2-phenyl-ethyl-amine;
3-(4-hydroxy-phenyl) propionic acid-(L-Glu)-(D-Arg)-2-phenyl-ethyl-amine;
3-(4-hydroxy-phenyl) propionic acid-(D-Glu)-(D-Arg)-2-phenyl-ethyl-amine;
p-hydroxybenzoic acid-(L-Arg)-aniline;
p-hydroxybenzoic acid-(D-Arg)-aniline;
p-hydroxybenzoic acid-(L-Glu)-aniline;
p-hydroxybenzoic acid-(D-Glu)-aniline;
p-hydroxybenzoic acid-(L-Arg)-benzylamine;
p-hydroxybenzoic acid-(D-Arg)-benzylamine;
p-hydroxybenzoic acid-(L-Glu)-benzylamine;
p-hydroxybenzoic acid-(D-Glu)-benzylamine;
p-hydroxybenzoic acid-(L-Arg)-2-phenyl-ethyl-amine;
p-hydroxybenzoic acid-(D-Arg)-2-phenyl-ethyl-amine;
p-hydroxybenzoic acid-(D-Glu)-2-phenyl-ethyl-amine;
p-hydroxybenzoic acid-(L-Glu)-2-phenyl-ethyl-amine;
2-(4-hydroxy-phenyl) acetic acid-(L-Arg)-aniline;
2-(4-hydroxy-phenyl) acetic acid-(D-Arg)-aniline;
2-(4-hydroxy-phenyl) acetic acid-(L-Glu)-aniline;
2-(4-hydroxy-phenyl) acetic acid-(D-Glu)-aniline;
2-(4-hydroxy-phenyl) acetic acid-(D-Arg)-benzylamine;
2-(4-hydroxy-phenyl) acetic acid-(L-Arg)-benzylamine;
2-(4-hydroxy-phenyl) acetic acid-(D-Glu)-benzylamine;
2-(4-hydroxy-phenyl) acetic acid-(L-Glu)-benzylamine;
2-(4-hydroxy-phenyl) acetic acid-(L-Arg)-2-phenyl-ethyl-amine;
2-(4-hydroxy-phenyl) acetic acid-(D-Arg)-2-phenyl-ethyl-amine;
2-(4-hydroxy-phenyl) acetic acid-(L-Glu)-2-phenyl-ethyl-amine;
2-(4-hydroxy-phenyl) acetic acid-(D-Glu)-2-phenyl-ethyl-amine;
3-(4-hydroxy-phenyl) propionic acid-(L-Arg)-aniline;
3-(4-hydroxy-phenyl) propionic acid-(D-Arg)-aniline;
3-(4-hydroxy-phenyl) propionic acid-(L-Glu)-aniline;
3-(4-hydroxy-phenyl) propionic acid-(D-Glu)-aniline;
3-(4-hydroxy-phenyl) propionic acid-(L-Arg)-benzylamine;
3-(4-hydroxy-phenyl) propionic acid-(D-Arg)-benzylamine;
3-(4-hydroxy-phenyl) propionic acid-(L-Glu)-benzylamine;
3-(4-hydroxy-phenyl) propionic acid-(D-Glu)-benzylamine;
3-(4-hydroxy-phenyl) propionic acid-(L-Arg)-2-phenyl-ethyl-amine;
3-(4-hydroxy-phenyl) propionic acid-(D-Arg)-2-phenyl-ethyl-amine;
3-(4-hydroxy-phenyl) propionic acid-(L-Glu)-2-phenyl-ethyl-amine; and
3-(4-hydroxy-phenyl) propionic acid-(D-Glu)-2-phenyl-ethyl-amine.

Alternatively, the moiety labelled as A′ in Formula I may be an oligopeptide having an amino acid sequence selected from the group listed directly above.

According to certain embodiments the A′ moiety is a peptide or peptoid moiety having the residues:
Xaa₁-Xaa₂-Xaa₃-Xaa₄ wherein:
Xaa₁ is L-Tyr, D-Tyr, N-methyl-L-Tyr, N-methyl-D-Tyr, p-hydroxybenzoic acid, 2-(4-hydroxy-phenyl) acetic acid, 3-(4-hydroxy-phenyl) propionic acid or acetyl;
Xaa₂ is L-Glu, D-Glu, L-Asp or D-Asp, N-methyl-L-Glu, N-methyl-D-Glu, N-methyl-L-Asp, N-methyl-D-Asp, L-Pro, D-Pro, N-methyl-L-Pro, N-methyl-D-Pro, L-Leu, D-Leu, N-methyl-L-Leu, N-methyl-D-Leu, or absent;
Xaa₃ is L-Arg, D-Arg, L-His or D-His, L-Lys, D-Lys, N-methyl-L-Arg, N-methyl-D-Arg, N-methyl-L-His, N-methyl-D-His, N-methyl-L-Lys, N-methyl-D-Lys, or absent; and
Xaa₄ is aniline, benzylamine, 2-phenyl-ethyl-amine, L-Phe or D-Phe, N-methyl-L-Phe, N-methyl-D-Phe, L-Trp, D-Trp, N-methyl-L-Trp, N-methyl-D-Trp.

According to certain embodiments either Xaa₂ or Xaa₃ are absent but not both Xaa₂ and Xaa₃. According to other embodiments Xaa₂ and Xaa₃ are both absent.

M may be simply an amide bond between adjacent peptide or peptoid moieties. Alternatively, it may be a molecular moiety introduced as a spacer and attached to adjacent peptide or peptoid moieties by amide bonds.

M may be an additional amino acid. Preferably it is an additional amino acid with a non-bulky side chain, for example glycine, alanine or serine or derivatives of any thereof. Alternatively M may be a non-amino acid moiety, for example, ε-aminocaproic acid, 3-amino-propionic acid, 4-amino-butyric acid. Other moieties can be methyl-amine, ethyl-amine, propyl-amine, butyl-amine, methylene, di-methylene, tri-methylene or tetra-methylene. In all cases M should be such that its presence does not materially interfere with binding between the A′ moiety and Gadd45β and/or MKK7. The extent of potential interference may be assessed by use of an in vitro binding assay.

### Oligomers and Multimers

Gadd45β/MKK7 inhibitors may encompass oligomers or multimers of molecules of the compound of formula I, said oligomers and multimers comprising two or more molecules of the compound of formula I each linked to a common scaffold moiety via an amide bond formed between an amine or carboxylic acid group present in molecules of the compound of formula I and an opposite amino or carboxylic acid group on a scaffold moiety said scaffold moiety participating in at least 2 amide bonds.

According to certain embodiments the common scaffold may be the amino acid lysine. Lysine is a tri-functional amino acid, having in addition to the functional groups which define it as an amino acid, an amino group on its side claim. This tri-functional nature allows it to form three amide bonds with peptides, peptoids or similar molecules. Other tri-functional amino acids which may be used as a common scaffold include D-α,β-diaminopropionic acid (D-Dap), L-α,β-diaminopropionic acid (L-Dap), L-α,δ-diaminobutyric acid (L-Dab), L-α,δ-diaminobutyric acid (L-Dab), and L-ornithine, D-ornithine. Other tri-functional non-standard amino acids may also be used. The common scaffold may also comprise branched peptides, peptoids or similar molecules which incorporate tri-functional amino acids within their sequence and have at least three functionally active terminal groups able to form amide bonds.

### Cell-Penetrating Peptides.

According to certain embodiments the compounds of formula I are conjugated to a cell penetrating peptide (CPP).

Such peptides may be attached to a compound of formula I either via one or more covalent bonds or by non-covalent associations.

CPPs may either directly penetrate the plasmalemma, for example the CPP may be Tat or a derivative, a peptide derived from the Antennapedia sequence, or a poly-arginine tag, a PTD-4 peptide, or a functionally equivalent cell-permeable peptide (Ho A, Schwarze S R, Mermelstein S J, Waksman G, Dowdy S F 2001 Synthetic protein transduction domains: enhanced transduction potential in vitro and in vivo. Cancer Res 61:474-477).

Alternatively, the CPP may enter the cell by mediating endocytosis or through mediating the formation of transitory membrane-spanning structures. For a discussion of cell penetrating peptides, the reader is directed to Wagstaff et al. (2006). Curr. Med. Chem. 13:171-1387 and references therein.

According to certain embodiments compounds may be conjugated to nano-particles (for example nano-Gold) in order to promote cellular uptake

### Fluorescent Dyes, Tag Moieties and Lipidated Derivatives.

Compounds of formula I may be conjugated to fluorescent dyes in order that their penetration into target tissues or cells may be monitored. Fluorescent dyes may be obtained with amino groups (i.e., succinimides, isothiocyanates, hydrazines), carboxyl groups (i.e., carbodiimides), thiol groups (i.e., maleimides and acetyl bromides) and azide groups which may be used to selectively react with the peptide moieties of compounds of formula I. Examples of fluorescent dyes include fluorescein and its derivatives, rhodamine and its derivatives.

Compounds of formula I may be conjugated to nanoparticles of discrete size such those described in Chithrani D B, Mol Membr Biol. 2010 Oct. 7, (Epub ahead of print) with a discrete size of up to 100 nm, whereby the peptides or their derivatives can be attached by a disulphide bridge to allow specific release within the reducing environment of the cytosol. Also peptide-nanoparticles conjugated via amide, ether, ester, thioether bonds can be used for the same purpose given the low toxicity of these compounds. Nanoparticles will favour cell uptake as well as will provide a mean to visualize and quantify cell uptake by fluorescence techniques (Schrand A M, Lin J B, Hens S C, Hussain S M., Nanoscale. 2010 Sep. 27, Epub ahead of print).

Tag moieties may be attached by similar means and similarly allow for monitoring of the success of targeting to tissues and cells.

Fatty acid derivatives of a compound comprising a compound of formula I linked to a fatty acid via a disulfide linkage may be used for delivery of an inhibitor compound to cells and tissues. Lipidisation markedly increases the absorption of the compounds relative to the rate of absorption of the corresponding unlipidised compounds, as well as prolonging blood and tissue retention of the compounds. Moreover, the disulfide linkage in lipidised derivative is relatively labile in the cells and thus facilitates intracellular release of the molecule from the fatty acid moieties. Suitable lipid-containing moieties are hydrophobic substituents with 4 to 26 carbon atoms, preferably 5 to 19 carbon atoms. Suitable lipid groups include, but are not limited to, the following: palmityl (C₁₅H₃i,), oleyl (C₁₅H₂₉), stearyl (C₁₇H₃₅), cholate; linolate, and deoxycholate.

### Ion Conjugates

Compounds of formula I may be functionally attached to metallic or radioactive ions. This attachment is typically achieved by the conjugation of an ion chelating agent (for example EDTA) which is chelated with the ion. By such means radioactive ions (for example ^{99m}Tc, ¹¹¹In, ⁶⁴Cu, ⁶⁷Cu, ⁸⁹Sr, ⁹⁰Y, ^{117m}Sn, ¹⁵³Sm, ¹⁸⁶Re, ¹⁸⁸Re, or ¹⁷⁷Lu) may be delivered to target cells as radiotherapy. Non-radioactive metallic ions (for example ions of gadolinium) may be used as a NMR-detectable marker.

Examples of preferred Gadd45β/MKK7 inhibitors of formula I are given below. Where the L/D configuration of an amino acid residue is not specified, both configurations are encompassed:

[SEQ ID NO: 55]
Acetyl-Tyr-Glu-Arg-Phe-NH₂;
para-hydroxybenzoic acid-Glu-Arg-aniline;
para-hydroxybenzoic acid-Glu-Arg-benzylamine;
para-hydroxybenzoic acid-Glu-Arg-2-phenyl-ethyl-
amine;
2-(4-hydroxyphenyl) acetic acid-Glu-Arg-aniline;
2-(4-hydroxyphenyl) acetic acid-Glu-Arg-
benzylamine;
2-(4-hydroxyphenyl) acetic acid-Glu-Arg-2-phenyl-
ethyl-amine;
3-(4-hydroxyphenyl) acetic acid-Glu-Arg-3-aniline;
3-(4-hydroxyphenyl) acetic acid-Glu-Arg-
benzylamine;
3-(4-hydroxyphenyl) acetic acid-Glu-Arg-2-phenyl-
ethyl-amine;
[SEQ ID NO.: 56]
Acetyl-Tyr-Asp-His-Phe-NH₂;
para-hydroxybenzoic-acid-Asp-His-aniline;
para-hydroxybenzoic-acid-Asp-His-benzylamine;
para-hydroxybenzoic-acid-Asp-His-3-phenyl-propyl-
amine;
2-(4-hydroxyphenyl) acetic acid-Asp-His-aniline;
2-(4-hydroxyphenyl) acetic acid-Asp-His-
benzylamine;
2-(4-hydroxyphenyl) acetic acid-Asp-His-2-phenyl-
ethyl-amine;
3-(4-hydroxyphenyl) propionic acid-Asp-His-
aniline;
3-(4-hydroxyphenyl) propionic acid-Asp-His-
benzylamine;
3-(4-hydroxyphenyl) propionic acid-Asp-His-2-
phenyl-ethyl-amine;
[SEQ ID NO.: 57]
Acetyl-Tyr-Asp-Lys-Phe-NH₂;
[SEQ ID NO.: 58]
Acetyl-Tyr-Glu-Lys-Phe-NH₂;
[SEQ ID NO.: 59]
Acetyl-Tyr-Glu-His-Phe-NH₂;
[SEQ ID NO.: 60]
Acetyl-Tyr-Asp-Arg-Phe-NH₂;
[SEQ ID NO.: 61]
Acetyl-Trp-Glu-His-Phe-NH₂;
[SEQ ID NO.: 62]
Acetyl-Trp-Glu-Lys-Phe-NH₂;
[SEQ ID NO.: 63]
Acetyl-Trp-Asp-His-Phe-NH₂;
[SEQ ID NO.: 64]
Acetyl-Trp-Asp-Lys-Phe-NH₂;
[SEQ ID NO.: 65]
Acetyl-Tyr-Glu-Arg-Tyr-NH₂;
[SEQ ID NO.: 66]
Acetyl-Tyr-Asp-Lys-Tyr-NH₂;
[SEQ ID NO.: 67]
Acetyl-Tyr-Glu-Lys-Tyr-NH₂;
[SEQ ID NO.: 68]
Acetyl-Tyr-Glu-His-Tyr-NH₂;
[SEQ ID NO.: 69]
Acetyl-Tyr-Asp-Arg-Tyr-NH₂;
[SEQ ID NO.: 70]
Acetyl-Trp-Glu-His-Tyr-NH₂;
[SEQ ID NO.: 71]
Acetyl-Trp-Glu-Lys-Tyr-NH₂;
[SEQ ID NO.: 72]
Acetyl-Trp-Asp-His-Tyr-NH₂;
[SEQ ID NO.: 73]
Acetyl-Trp-Asp-Lys-Tyr-NH₂;
[SEQ ID NO.: 74]
internal lactam of Acetyl-Tyr-Glu-Lys-Phe-NH₂;
[SEQ ID NO.: 75]
Acetyl-Tyr-Gln-Arg-Phe-NH₂;
[SEQ ID NO.: 76]
Acetyl-Tyr-Met-Arg-Phe-NH₂;
[SEQ ID NO.: 77]
Acetyl-Tyr-Leu-Arg-Phe-NH₂;
Acetyl-Tyr-Arg-Phe-NH₂;
Acetyl-Tyr-Arg-Tyr-NH₂;
Acetyl-Tyr-Glu-Phe-NH₂;
Acetyl-Tyr-Glu-Tyr-NH₂;
Acetyl-Tyr-Asp-Phe-NH₂;
Acetyl-Tyr-Asp-Tyr-NH₂;
Acetyl-Tyr-Pro-Phe-NH₂;
Acetyl-Tyr-Lys-Phe-NH₂;
Acetyl-Tyr-His-Phe-NH₂;
H-Tyr-Arg-Phe-NH₂;
H-Tyr-Arg-Tyr-NH₂;
H-Tyr-Glu-Phe-NH₂;
H-Tyr-Glu-Tyr-NH₂;
H-Tyr-Asp-Phe-NH₂;
H-Tyr-Asp-Tyr-NH₂;
H-Tyr-Pro-Phe-NH₂;
H-Tyr-Lys-Phe-NH₂;
H-Tyr-His-Phe-NH₂;
Benzyloxycarbonyl-Tyr-Arg-Phe-NH₂;
Benzyloxycarbonyl-Tyr-Arg-Tyr-NH₂;
Benzyloxycarbonyl-Tyr-Glu-Phe-NH₂;
Benzyloxycarbonyl-Tyr-Glu-Tyr-NH₂;
Benzyloxycarbonyl-Tyr-Asp-Phe-NH₂;
Benzyloxycarbonyl-Tyr-Asp-Tyr-NH₂;
Benzyloxycarbonyl-Tyr-Pro-Phe-NH₂;
Benzyloxycarbonyl-Tyr-Lys-Phe-NH₂;
Benzyloxycarbonyl-Tyr-His-Phe-NH₂;
[SEQ ID NO.: 78]
Benzyloxycarbonyl-Tyr-Glu-Arg-Phe-NH₂;
[SEQ ID NO.: 79]
Benzyloxycarbonyl-Tyr-Asp-His-Phe-NH₂;
[SEQ ID NO.: 80]
Benzyloxycarbonyl-Tyr-Asp(OMe)-His-Phe-NH₂;
Benzyloxycarbonyl-Tyr-Arg-Phe-NH₂;
Benzyloxycarbonyl-Tyr-Glu-Phe-NH₂;
Benzyloxycarbonyl-(N-methyl)Tyr-(N-methyl)Arg-(N-
methyl)Phe-NH₂;
Benzyloxycarbonyl-(N-methyl)Tyr-Glu-(N-methyl)Phe-
NH₂;
Benzyloxycarbonyl-Tyr-(N-methyl)Arg-(N-methyl)Phe-
NH₂;
Benzyloxycarbonyl-(N-methyl)Tyr-(N-methyl)Arg-Phe-
NH₂;
Benzyloxycarbonyl-Tyr-Glu-(N-methyl)Phe-NH₂;
Benzyloxycarbonyl-Tyr-(N-methyl)Glu-Phe-NH₂;
Benzyloxycarbonyl-(N-methyl)Tyr-Glu-Phe-NH₂;
Acetyl-(N-methyl)Tyr-(N-methyl)Arg-(N-methyl)Phe-
NH₂;
Acetyl-(N-methyl)Tyr-Glu-(N-methyl)Phe-NH₂;
Acetyl-Tyr-(N-methyl)Arg-(N-methyl)Phe-NH₂;
Acetyl-(N-methyl)Tyr-(N-methyl)Arg-Phe-NH₂;
Acetyl-Tyr-Glu-(N-methyl)Phe-NH₂;
Acetyl-Tyr-(N-methyl)Glu-Phe-NH₂;
Acetyl-(N-methyl)Tyr-Glu-Phe-NH₂;
H-(N-methyl)Tyr-(N-methyl)Arg-(N-methyl)Phe-NH₂;
H-(N-methyl)Tyr-Glu-(N-methyl)Phe-NH₂;
H-Tyr-(N-methyl)Arg-(N-methyl)Phe-NH₂;
H-(N-methyl)Tyr-(N-methyl)Arg-Phe-NH₂;
H-Tyr-Glu-(N-methyl)Phe-NH₂;
H-Tyr-(N-methyl)Glu-Phe-NH₂;
H-(N-methyl)Tyr-Glu-Phe-NH₂;
Acetyl-Tyr-Glu-(β-homo)Phe-NH₂;
Acetyl-Tyr-(β-homo)Glu-Phe-NH₂;
Acetyl-(β-homo)Tyr-Glu-Phe-NH₂;
Acetyl-Tyr-Phe-NH₂;
Acetyl-Phe-Tyr-NH₂;
Benzyloxycarbonyl-Tyr-Phe-NH₂;
Benzyloxycarbonyl-Phe-Tyr-NH₂;
H-Tyr-Phe-NH₂;
H-Phe-Tyr-NH₂;
[SEQ ID NO.: 81]
(3-Methoxy-4-hydroxy-benzoyl)-Tyr-Glu-Arg-Phe-NH₂;
[SEQ ID NO.: 82]
(3-Methoxy-4-hydroxy-benzoyl)-Tyr-Asp-His-Phe-NH₂;
[SEQ ID NO.: 83]
(3-Methoxy-4-hydroxy-benzoyl)-Tyr-Asp(OMe)-His-
Phe-NH₂;
(3-Methoxy-4-hydroxy-benzoyl)-Tyr-Arg-Phe-NH₂;
(3-Methoxy-4-hydroxy-benzoyl)-Tyr-Glu-Phe-NH₂;
[SEQ ID NO.: 84]
Fluorenylmethyloxycarbonyl-Tyr-Glu-Arg-Phe-NH₂;
[SEQ ID NO.: 85]
Fluorenylmethyloxycarbonyl-Tyr-Asp-His-Phe-NH₂;
[SEQ ID NO.: 86]
Fluorenylmethyloxycarbonyl-Tyr-Asp(OMe)-His-Phe-
NH₂;
[SEQ ID NO.: 87]
Fluorenylmethyloxycarbonyl-Tyr-Asp(OMe)-His-Phe-
NH₂;
Fluorenylmethyloxycarbonyl-Tyr-Arg-Phe-NH₂;
Fluorenylmethyloxycarbonyl-Tyr-Glu-Phe-NH₂;
[SEQ ID NO.: 88]
Myristyl-Tyr-Glu-Arg-Phe-NH₂;
[SEQ ID NO.: 89]
Myristyl-Tyr-Asp-His-Phe-NH₂;
Myristyl-Tyr-Arg-Phe-NH₂;
Myristyl-Tyr-Glu-Phe-NH₂;
[SEQ ID NO.: 90]
Myristyl-Tyr-Asp(OMe)-His-Phe-NH₂;
[SEQ ID NO.: 91]
Acetyl-Tyr-Glu-Arg-Phe-Gly-Tyr-Glu-Arg-Phe-NH₂;
[SEQ ID NO.: 92]
Acetyl-Tyr-Asp-His-Phe-Gly-Tyr-Asp-His-Phe-NH₂;
[SEQ ID NO.: 93]
Acetyl-Tyr-Arg-Phe-Gly-Tyr-Arg-Phe-NH₂;
[SEQ ID NO.: 94]
Acetyl-Tyr-Asp(OMe)-His-Phe-Gly-Tyr-Asp(OMe)-His-
Phe-NH₂;
[SEQ ID NO.: 95]
Benzyloxycarbonyl-Tyr-Glu-Arg-Phe-Gly-Tyr-Glu-Arg-
Phe-NH₂;
[SEQ ID NO.: 96]
Benzyloxycarbonyl-Tyr-Asp-His-Phe-Gly-Tyr-Asp-His-
Phe-NH₂;
[SEQ ID NO.: 97]
Benzyloxycarbonyl-Tyr-Arg-Phe-Gly-Tyr-Arg-Phe-NH₂;
and
[SEQ ID NO.: 98]
Benzyloxycarbonyl-Tyr-Asp(OMe)-His-Phe-Gly-Tyr-Asp
(OMe)-His-Phe-NH₂;

Further examples of Gadd45β/MKK7 inhibitors include:

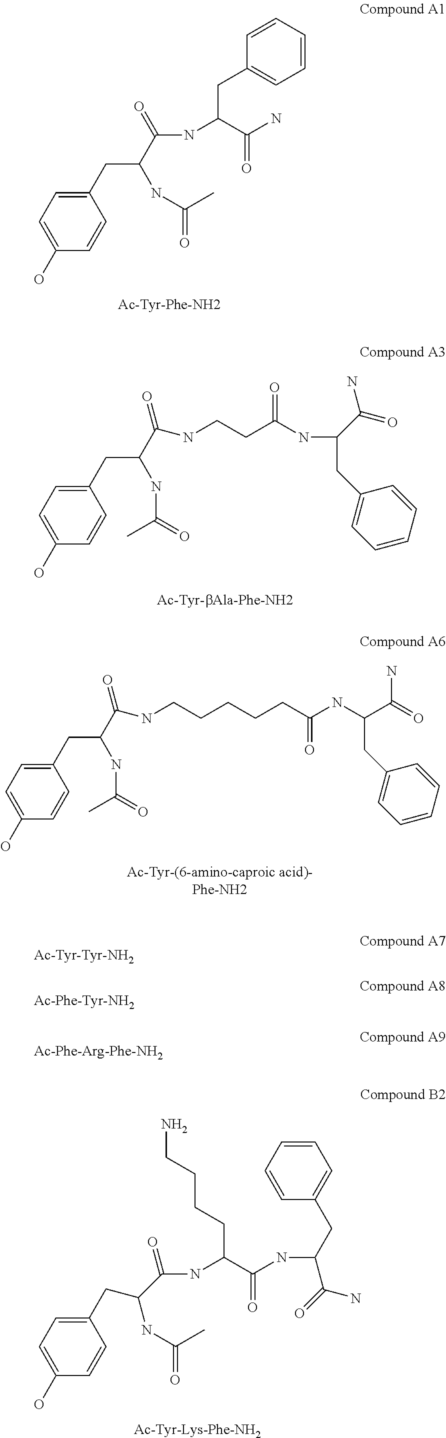

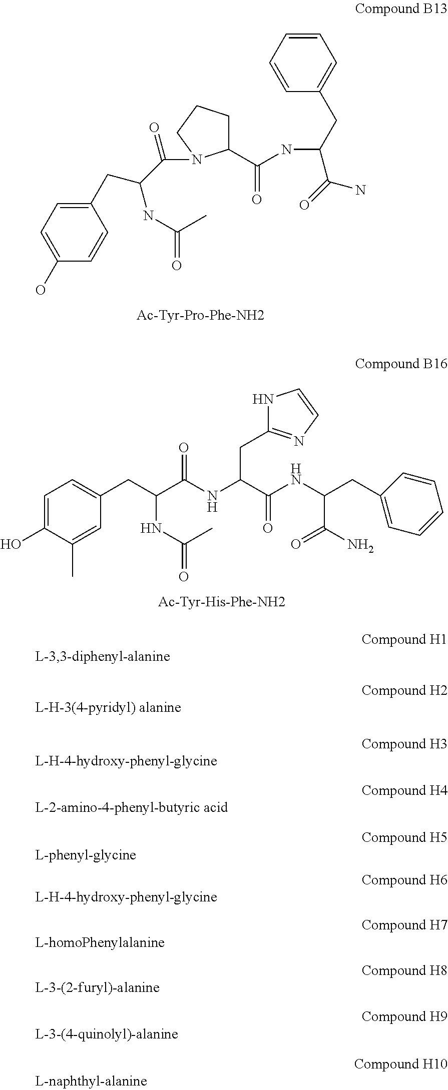 [SMILES: "Ac-Tyr-His-Phe-NH2" ; "Ac-Tyr-Pro-Phe-NH2" ; CC(=O)NC(Cc1ccc(O)c(C)c1)C(=O)NC(Cc1ncc[nH]1)C(=O)NC(Cc1ccccc1)C(N)=O ; CC(=O)NC(Cc1ccc(O)cc1)C(=O)N1CCCC1C(=O)NC(Cc1ccccc1)C(N)=O]

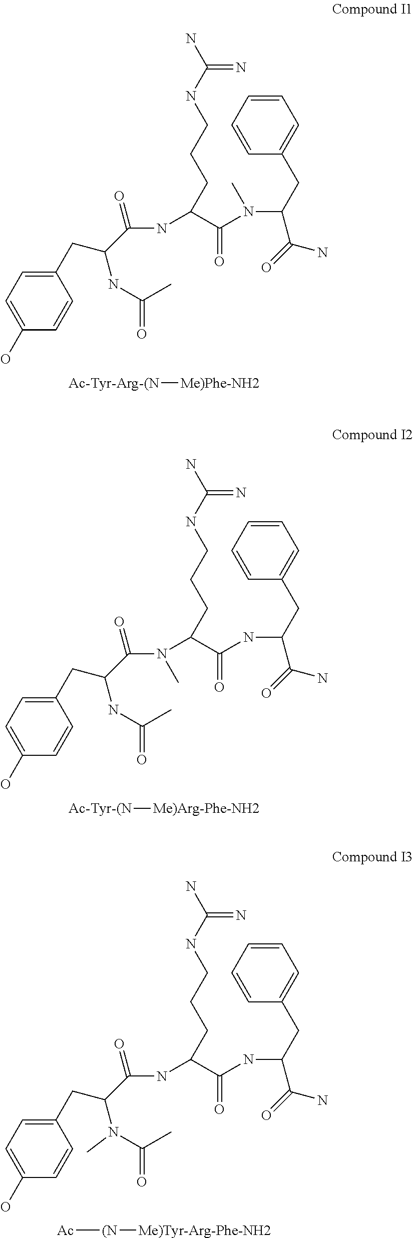

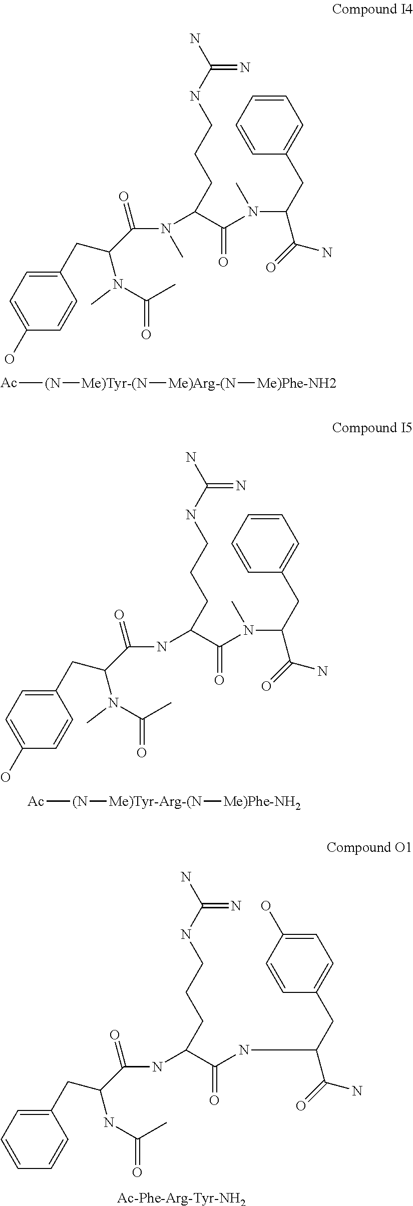

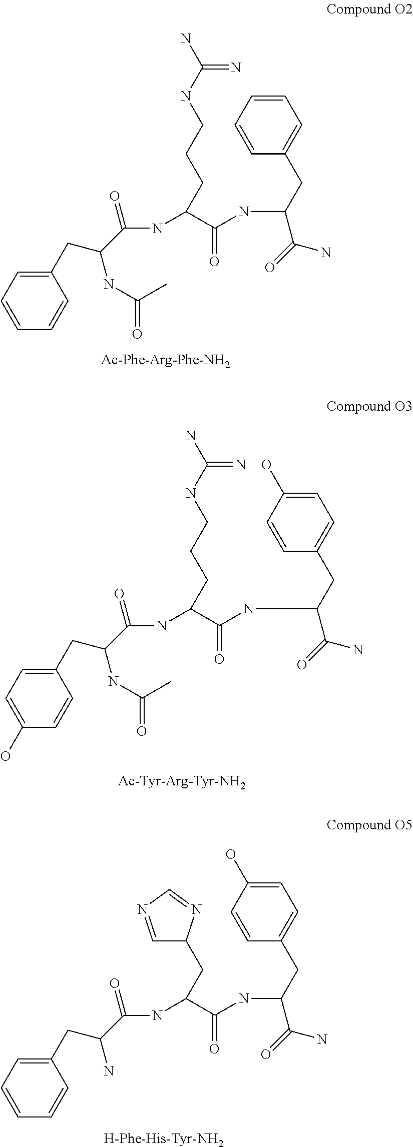 [SMILES: "H-Phe-His-Tyr-NH2" ; "Ac-Phe-Arg-Phe-NH2" ; "Ac-Tyr-Arg-Tyr-NH2" ; CC(=O)NC(Cc1ccccc1)C(=O)NC(CCCNC(=N)N)C(=O)NC(Cc1ccccc1)C(N)=O ; NC(=O)C(Cc1ccc(O)cc1)NC(=O)C(CC1C=NC=N1)NC(=O)C(N)Cc1ccccc1 ; CC(=O)NC(Cc1ccc(O)cc1)C(=O)NC(CCCNC(=N)N)C(=O)NC(Cc1ccc(O)cc1)C(N)=O]

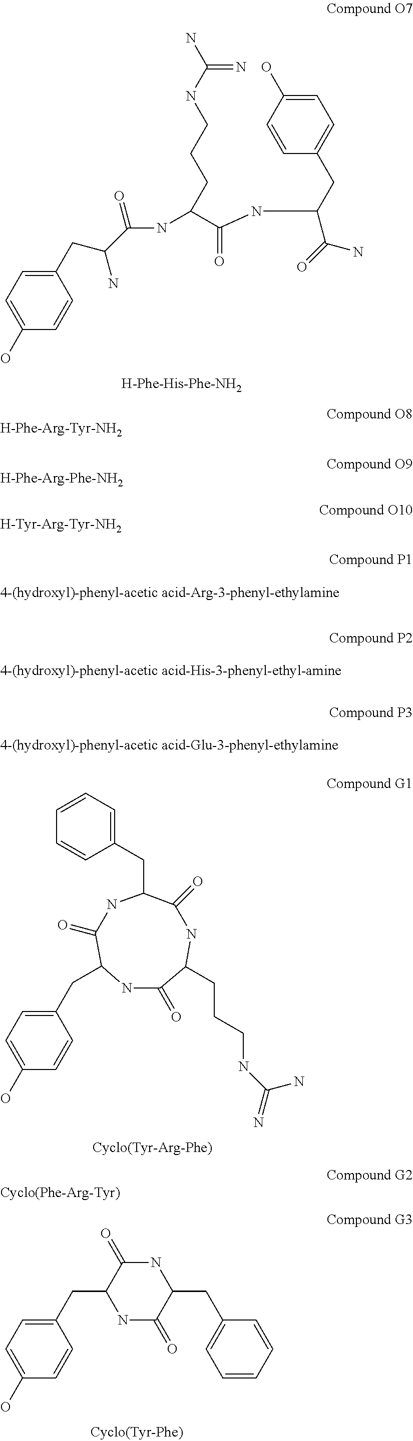

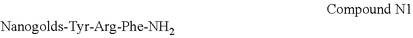

According to certain embodiments, compounds disclosed specifically herein, including in the examples, are preferred compounds or are preferred embodiments of the A′ moiety of formula I. Multimer versions or the specific compounds explicitly disclosed herein may be used. For example the 3 or 4 residue peptide or peptoid moieties of the specific compounds disclosed herein may correspond to the A, A′, A″, A′″ or A″″ moiety of compounds of formula I.

In a particularly preferred embodiment, the Gadd45β/MKK7 inhibitor is Acetyl-Tyr-Arg-Phe-NH₂, or a derivative thereof, or a salt thereof, including a salt of a derivative thereof. In an especially preferred embodiment, the Gadd45β/MKK7 inhibitor is DTP3, or a derivative thereof, or a salt thereof, including a salt of a derivative thereof, wherein DTP3 has the structure shown below:

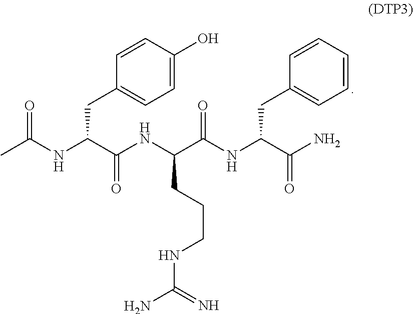 [SMILES: CC(=O)N[C@H](Cc1ccc(O)cc1)C(=O)N[C@H](CCCNC(=N)N)C(=O)N[C@H](Cc1ccccc1)C(N)=O]

In other words, DTP3 is Acetyl-Tyr-Arg-Phe-NH₂ in which all three of the amino acid residues are D-amino acid residues. In a particularly preferred embodiment, the Gadd45β/MKK7 inhibitor is DTP3, or a salt thereof.

### Haematological Malignancies

The disorders treated by the present invention are either characterised by i) high or increased expression or activity of Gadd45β and/or MKK7 or ii) are characterised by aberrant activation of the NF-κB pathway and are amenable to treatment by the induction of programmed cell death by the inhibition of Gadd45β activity and/or activation of MKK7. Haematological malignancies treated by the present invention may be lymphomas or leukaemias. According to certain embodiments the invention may relate to haematological malignancies wherein said malignancy is a member of one or more of the following groups:
1. Lymphoma
2. Leukaemia
3. Mature B-cell malignancies
4. Mature T-cell malignancies
5. Mature natural killer cell malignancies
6. Hodgkin's lymphoma
7. Chronic lymphocytic leukaemia
8. Acute lymphocytic leukaemia
9. Chronic myelogenous leukaemia
10. Acute myelogenous leukaemia

According to certain preferred embodiments the invention may relate to a haematological malignancy selected from one of the following groups:

myeloproliferative neoplasms (such as chronic myeloid leukaemia, chronic myeloproliferative neoplasms, primary myelofibrosis); myelodysplastic/myeloproliferative neoplasms; myelodysplastic syndromes; acute myeloid leukaemias (such as acute myeloid leukaemia, acute promyelocytic leukaemia); precursor lymphoid neoplasms (such as B-lymphoblastic leukaemia, T-lymphoblastic leukaemia); and mature lymphoid neoplasms (such as Monoclonal B-cell lymphocytosis, chronic lymphocytic leukaemia, hairy cell leukaemia, lymphoproliferative disorder NOS, monoclonal gammopathy of undetermined significance, plasmacytoma, myeloma, marginal zone lymphoma, follicular lymphoma, mantle cell lymphoma, diffuse large B-cell lymphoma, Burkitt's lymphoma, classical Hodgkin's lymphoma, lymphocyte predominant nodular Hodgkin's lymphoma, T-cell lymphoma, T-cell leukaemias).

According to certain preferred embodiments the invention may relate to a haematological malignancy selected from one of the following groups:
1. Burkitt's lymphoma, B-cell leukaemia, diffuse large B-cell lymphoma (DCBCL) T-cell leukaemia, acute myelogenic leukaemia (AML), acute lymphoblastic leukaemia (ALL), multiple myeloma (MM), small lymphocytic lymphoma (SLL), mantle cell lymphoma, marginal zone lymphoma, follicular lymphoma, MALT lymphoma, non-Hodgkin's lymphoma (NHL), Hodgkin's lymphoma (HL), hairy cell lymphoma (HCL), adult T-cell leukaemia, chronic lymphatic leukaemia (CLL), chronic myeloid leukaemia (CIVIL), cutaneous T-cell lymphoma, myelodysplastic syndrome and pro-monocytic leukaemia.
2. Burkitt's lymphoma, diffuse large B-cell lymphoma (DCBCL), T-cell leukaemia, acute myelogenic leukaemia (AML), acute lymphocytic leukaemia (ACL), multiple myeloma (MM), mantle cell lymphoma, marginal zone lymphoma, follicular lymphoma, non-Hodgkin's lymphoma (NHL), MALT lymphoma, Hodgkin's lymphoma (HL), adult T-cell leukaemia, chronic lymphatic leukaemia (CLL), chronic myeloid leukaemia (CML), cutaneous T-cell lymphoma, myelodysplastic syndrome and pro-monocytic leukaemia.
3. Promonocytic leukaemia, Burkitt's lymphoma, diffuse large B-cell lymphoma (DLBCL), mantle cell lymphoma, marginal zone lymphoma, follicular lymphoma, MALT lymphoma, non-Hodgkin's lymphoma NHL), Hodgkin's lymphoma (HL), B-cell leukaemia, multiple myeloma, chronic myeloid leukaemia (CIVIL).
4. Burkitt's lymphoma, diffuse large B-cell lymphoma, multiple myeloma, MALT lymphoma.
5. Diffuse large B-cell lymphoma, multiple myeloma, MALT lymphoma.
6. Diffuse large B-cell lymphoma, multiple myeloma, Hodgkin's lymphoma.

In one particularly preferred embodiment, the haematological malignancy is multiple myeloma. In one particularly preferred embodiment, the haematological malignancy is diffuse large B-cell lymphoma (DLBCL). In one particularly preferred embodiment, the haematological malignancy is MALT lymphoma. In one particularly preferred embodiment, the haematological malignancy is Hodgkin's lymphoma.

In the first aspect of the invention, the haematological malignancy is a resistant haematological malignancy. As discussed above, current therapies for haematological malignancies such as multiple myeloma are poorly effective in some patients. Patients may acquire or possess inherent resistance to treatment with standard therapies. For example, patients may not respond to therapy at all, or may only respond briefly and may then relapse. Accordingly, a resistant haematological malignancy is a haematological malignancy which is resistant to a therapy indicated for said haematological malignancy (such as an anti-cancer agent indicated for said haematological malignancy), and which is other than a Gadd45β/MKK7 inhibitor; more preferably a resistant haematological malignancy is a haematological malignancy which is resistant to therapy with an anti-cancer agent which exerts effects via the NF-κB pathway and which is other than a Gadd45β/MKK7 inhibitor.

As demonstrated herein, the Gadd45β/MKK7 inhibitor DTP3 has been shown to retain efficacy against cell lines resistant to a number of current therapies for multiple myeloma, i.e. cell lines resistant to the proteasome inhibitor bortezomib, to the IMiD lenalidomide (whose effects include downregulation of NF-κB) or to the glucocorticoid dexamethasone (which also inhibits the NF-κB pathway). The mechanism by which the Gadd45β/MKK7 complex inhibits apoptosis in cancer cells operates downstream of the proteasome, cerebron, BTK, PI3K, PKC, SYK, CARD11, TAK1, A20, CD79B/A, MALT1, MYD88 and the NF-κB pathway. The mechanism by which the Gadd45β/MKK7 complex inhibits apoptosis in cancer cells also operates downstream and independently of CD38, CD20 and SLAM7, down-regulation of which is understood to be associated with resistance. Accordingly, given the observed results in resistant cells, and given the difference between the mechanism of action by which Gadd45β/MKK7 inhibitors and other anti-cancer agents operate, Gadd45β/MKK7 inhibitors are expected to retain full anti-cancer activity in cancer cells, such as multiple myeloma cells, DLBCL cells, Hodgkin's lymphoma cells and MALT lymphoma cells, which are resistant to anti-cancer agents exerting their effects through these therapeutic targets.

In one preferred embodiment, the resistant haematological malignancy is a haematological malignancy which is resistant to therapy with an anti-cancer agent which is indicated for said haematological malignancy selected from the group consisting of a proteasome inhibitor (e.g. bortezomib, carfilzomib), an IMiD anti-cancer agent (e.g. lenalidomide, thalidomide, pomalidomide), a glucocorticoid (e.g. dexamethasone, prednisolone, prednisone), an anti-CD38 agent (e.g. daratumamab, for example an anti-CD 38 agent in combination with lenalidomide), an anti-SLAM7 agent (e.g. elotuzumab), a Bruton's tyrosine kinase inhibitor (e.g. ibrutinib), a protein kinase C inhibitor (e.g. sotrastaurin), an anti-CD20 agent (e.g. rituximab), a cytotoxic agent (e.g. cyclophosphamide), an alkylating agent (e.g. melphalan), an anthracycline antibiotic anti-cancer agent (e.g. doxorubicin), a mitotic inhibitor (e.g. vincristine), a purine analogue (e.g. fludarabine), a PI3Kdelta inhibitor (e.g. idelasib) and a Bcl-2 inhibitor (e.g. ABT-199). The resistant haematological malignancy may be a haematological malignancy which is resistant to therapy with a histone deacetylase (HDAC) inhibitor (eg Panobinostat).

In one preferred embodiment, the resistant haematological malignancy is multiple myeloma which is resistant to treatment with an anti-cancer agent selected from the group consisting of a proteasome inhibitor (e.g. bortezomib, carfilzomib), an IMiD anti-cancer agent (e.g. lenalidomide, thalidomide, pomalidomide), a glucocorticoid (e.g. dexamethasone, prednisolone, prednisone), a cytotoxic agent (e.g. cyclophosphamide), an alkylating agent (e.g. melphalan), an anthracycline antibiotic anti-cancer agent (e.g. doxorubicin), a mitotic inhibitor (e.g. vincristine), an anti-CD38 agent (e.g. daratumamab), and an anti-SLAM7 agent (e.g. elotuzumab). More preferably, the resistant haematological malignancy is multiple myeloma which is resistant to treatment with an anti-cancer agent selected from the group consisting of a proteasome inhibitor (e.g. bortezomib, carfilzomib), an IMiD anti-cancer agent (e.g. lenalidomide, thalidomide, pomalidomide), a glucocorticoid (e.g. dexamethasone, prednisolone, prednisone), a cytotoxic agent (e.g. cyclophosphamide), an anti-cancer alkylating agent (e.g. melphalan), an anthracycline antibiotic anti-cancer agent (e.g. doxorubicin) and a mitotic inhibitor (e.g. vincristine). In one preferred embodiment, the resistant haematological malignancy is multiple myeloma which is resistant to treatment with an anti-cancer agent selected from the group consisting of a proteasome inhibitor (e.g. bortezomib, carfilzomib), an IMiD anti-cancer agent (e.g. lenalidomide, thalidomide, pomalidomide), and a glucocorticoid (e.g. dexamethasone, prednisolone, prednisone).

In one preferred embodiment, the resistant haematological malignancy is multiple myeloma which is resistant to treatment with a proteasome inhibitor, more preferably the resistant haematological malignancy is bortezomib-resistant multiple myeloma, most preferably the resistant haematological malignancy is bortezomib-resistant multiple myeloma and the Gadd45β/MKK7 inhibitor is DTP3. In one preferred embodiment, the resistant haematological malignancy is multiple myeloma which is resistant to treatment with an IMiD anti-cancer agent, more preferably the resistant haematological malignancy is lenalidomide-resistant multiple myeloma, most preferably the resistant haematological malignancy is lenalidomide-resistant multiple myeloma and the Gadd45β/MKK7 inhibitor is DTP3. In one preferred embodiment the resistant haematological malignancy is multiple myeloma which is resistant to treatment with a glucocorticoid, more preferably the resistant haematological malignancy is dexamethasone-resistant multiple myeloma, most preferably the resistant haematological malignancy is dexamethasone-resistant multiple myeloma and the Gadd45β/MKK7 inhibitor is DTP3. In one preferred embodiment the resistant haematological malignancy is multiple myeloma which is resistant to treatment with a cytotoxic agent, more preferably the resistant haematological malignancy is cyclophosphamide-resistant multiple myeloma, most preferably the resistant haematological malignancy is cyclophosphamide-resistant multiple myeloma and the Gadd45β/MKK7 inhibitor is DTP3. In one preferred embodiment the resistant haematological malignancy is multiple myeloma which is resistant to treatment with an anti-cancer alkylating agent, more preferably the resistant haematological malignancy is melphalan-resistant multiple myeloma, most preferably the resistant haematological malignancy is melphalan-resistant multiple myeloma and the Gadd45β/MKK7 inhibitor is DTP3. In one preferred embodiment the resistant haematological malignancy is multiple myeloma which is resistant to treatment with an anthracycline antibiotic anti-cancer agent, more preferably the resistant haematological malignancy is doxorubicin-resistant multiple myeloma, most preferably the resistant haematological malignancy is doxorubicin-resistant multiple myeloma and the Gadd45β/MKK7 inhibitor is DTP3. In one preferred embodiment the resistant haematological malignancy is multiple myeloma which is resistant to treatment with a mitotic inhibitor, more preferably the resistant haematological malignancy is vincristine-resistant multiple myeloma, most preferably the resistant haematological malignancy is vincristine-resistant multiple myeloma and the Gadd45β/MKK7 inhibitor is DTP3.

In one preferred embodiment, the resistant haematological malignancy is multiple myeloma which is resistant to treatment with a combination of i) bortezomib, and ii) dexamethasone. In one preferred embodiment, the resistant haematological malignancy is multiple myeloma which is resistant to treatment with a combination of i) bortezomib, and ii) dexamethasone, and the Gadd45β/MKK7 inhibitor is DTP3. In one preferred embodiment, the resistant haematological malignancy is multiple myeloma which is resistant to treatment with a combination of i) lenalidomide, and ii) dexamethasone. In one preferred embodiment, the resistant haematological malignancy is multiple myeloma which is resistant to treatment with a combination of i) lenalidomide, and ii) dexamethasone, and the Gadd45β/MKK7 inhibitor is DTP3. In one preferred embodiment, the resistant haematological malignancy is multiple myeloma which is resistant to treatment with a combination of i) bortezomib, ii) lenalidomide or thalidomide, and iii) dexamethasone. In one preferred embodiment, the resistant haematological malignancy is multiple myeloma which is resistant to treatment with a combination of i) bortezomib, ii) lenalidomide or thalidomide, and iii) dexamethasone, and the Gadd45β/MKK7 inhibitor is DTP3. In one preferred embodiment, the resistant haematological malignancy is multiple myeloma which is resistant to treatment with a combination of i) bortezomib, ii) cyclophosphamide, and iii) dexamethasone. In one preferred embodiment, the resistant haematological malignancy is multiple myeloma which is resistant to treatment with a combination of i) bortezomib, ii) cyclophosphamide, and iii) dexamethasone, and the Gadd45β/MKK7 inhibitor is DTP3. In one preferred embodiment, the resistant haematological malignancy is multiple myeloma which is resistant to treatment with a combination of i) bortezomib, ii) lenalidomide, iii) cyclophosphamide, and iv) dexamethasone. In one preferred embodiment, the resistant haematological malignancy is multiple myeloma which is resistant to treatment with a combination of i) bortezomib, ii) lenalidomide, iii) cyclophosphamide, and iv) dexamethasone, and the Gadd45β/MKK7 inhibitor is DTP3. In one preferred embodiment, the resistant haematological malignancy is multiple myeloma which is resistant to treatment with a combination of i) lenalidomide, ii) cyclophosphamide, and iii) dexamethasone. In one preferred embodiment, the resistant haematological malignancy is multiple myeloma which is resistant to treatment with a combination of i) lenalidomide, ii) cyclophosphamide, and iii) dexamethasone, and the Gadd45β/MKK7 inhibitor is DTP3. In one preferred embodiment, the resistant haematological malignancy is multiple myeloma which is resistant to treatment with a combination of i) bortezomib, ii) melphalan, and iii) prednisone. In one preferred embodiment, the resistant haematological malignancy is multiple myeloma which is resistant to treatment with a combination of i) bortezomib, ii) melphalan, and iii) prednisone, and the Gadd45β/MKK7 inhibitor is DTP3. In one preferred embodiment, the resistant haematological malignancy is multiple myeloma which is resistant to treatment with a combination of i) bortezomib, ii) doxorubicin, and iii) dexamethasone. In one preferred embodiment, the resistant haematological malignancy is multiple myeloma which is resistant to treatment with a combination of i) bortezomib, ii) doxorubicin, and iii) dexamethasone, and the Gadd45β/MKK7 inhibitor is DTP3. In one preferred embodiment, the resistant haematological malignancy is multiple myeloma which is resistant to treatment with a combination of i) melphalan, ii) prednisone, and iii) thalidomide. In one preferred embodiment, the resistant haematological malignancy is multiple myeloma which is resistant to treatment with a combination of i) melphalan, ii) prednisone, and iii) thalidomide, and the Gadd45β/MKK7 inhibitor is DTP3. In one preferred embodiment, the resistant haematological malignancy is multiple myeloma which is resistant to treatment with a combination of i) melphalan, and ii) prednisone. In one preferred embodiment, the resistant haematological malignancy is multiple myeloma which is resistant to treatment with a combination of i) melphalan, and ii) prednisone, and the Gadd45β/MKK7 inhibitor is DTP3. In one preferred embodiment, the resistant haematological malignancy is multiple myeloma which is resistant to treatment with a combination of i) vincristine, ii) doxorubicin, and iii) dexamethasone. In one preferred embodiment, the resistant haematological malignancy is multiple myeloma which is resistant to treatment with a combination of i) vincristine, ii) doxorubicin, and iii) dexamethasone, and the Gadd45β/MKK7 inhibitor is DTP3.

In one preferred embodiment, the resistant haematological malignancy is diffuse large B-cell lymphoma which is resistant to treatment with an anti-cancer agent selected from the group consisting of a Bruton's tyrosine kinase inhibitor (e.g. ibrutinib), a protein kinase C inhibitor (e.g. sotrastaurin), an anti-CD20 agent (e.g. rituximab), a cytotoxic agent (e.g. cyclophosphamide), an anthracycline antibiotic anti-cancer agent (e.g. doxorubicin), a mitotic inhibitor (e.g. vincristine), a glucocorticoid (e.g. prednisone), a purine analogue (e.g. fludarabine), a PI3Kdelta inhibitor (e.g. idelasib) and a Bcl-2 inhibitor (e.g. ABT-199). In one preferred embodiment the resistant haematological malignancy is diffuse large B-cell lymphoma (DLBCL) which is resistant to treatment with an anti-cancer agent selected from the group consisting of an anti-CD20 agent (e.g. rituximab), a cytotoxic agent (e.g. cyclophosphamide), an anthracycline antibiotic anti-cancer agent (e.g. doxorubicin), a mitotic inhibitor (e.g. vincristine), a glucocorticoid (e.g. prednisone) and a Bruton's tyrosine kinase inhibitor (e.g. ibrutinib). In one preferred embodiment the resistant haematological malignancy is diffuse large B-cell lymphoma which is resistant to treatment with a Bruton's tyrosine kinase inhibitor, more preferably the resistant haematological malignancy is ibrutinib-resistant diffuse large B-cell lymphoma, most preferably the resistant haematological malignancy is ibrutinib-resistant diffuse large B-cell lymphoma and the Gadd45β/MKK7 inhibitor is DTP3. In one preferred embodiment the resistant haematological malignancy is diffuse large B-cell lymphoma which is resistant to treatment with a protein kinase C inhibitor, more preferably the resistant haematological malignancy is sotrastaurin-resistant diffuse large B-cell lymphoma, most preferably the resistant haematological malignancy is sotrastaurin-resistant diffuse large B-cell lymphoma and the Gadd45β/MKK7 inhibitor is DTP3. In one preferred embodiment the resistant haematological malignancy is diffuse large B-cell lymphoma which is resistant to treatment with an anti-CD20 agent, more preferably the resistant haematological malignancy is rituximab-resistant diffuse large B-cell lymphoma, most preferably the resistant haematological malignancy is rituximab-resistant diffuse large B-cell lymphoma and the Gadd45β/MKK7 inhibitor is DTP3. In one preferred embodiment the resistant haematological malignancy is diffuse large B-cell lymphoma which is resistant to treatment with a cytotoxic agent, more preferably the resistant haematological malignancy is cyclophosphamide-resistant diffuse large B-cell lymphoma, most preferably the resistant haematological malignancy is cyclophosphamide-resistant diffuse large B-cell lymphoma and the Gadd45β/MKK7 inhibitor is DTP3. In one preferred embodiment the resistant haematological malignancy is diffuse large B-cell lymphoma which is resistant to treatment with an anthracycline antibiotic anti-cancer agent, more preferably the resistant haematological malignancy is doxorubicin-resistant diffuse large B-cell lymphoma, most preferably the resistant haematological malignancy is doxorubicin-resistant diffuse large B-cell lymphoma and the Gadd45β/MKK7 inhibitor is DTP3. In one preferred embodiment the resistant haematological malignancy is diffuse large B-cell lymphoma which is resistant to treatment with a mitotic inhibitor, more preferably the resistant haematological malignancy is vincristine-resistant diffuse large B-cell lymphoma, most preferably the resistant haematological malignancy is vincristine-resistant diffuse large B-cell lymphoma and the Gadd45β/MKK7 inhibitor is DTP3. In one preferred embodiment the resistant haematological malignancy is diffuse large B-cell lymphoma which is resistant to treatment with a glucocorticoid, more preferably the resistant haematological malignancy is prednisone-resistant diffuse large B-cell lymphoma, most preferably the resistant haematological malignancy is prednisone-resistant diffuse large B-cell lymphoma and the Gadd45β/MKK7 inhibitor is DTP3. In one preferred embodiment the resistant haematological malignancy is diffuse large B-cell lymphoma which is resistant to treatment with a purine analogue, more preferably the resistant haematological malignancy is fludarabine-resistant diffuse large B-cell lymphoma, most preferably the resistant haematological malignancy is fludarabine-resistant diffuse large B-cell lymphoma and the Gadd45β/MKK7 inhibitor is DTP3. In one preferred embodiment the resistant haematological malignancy is diffuse large B-cell lymphoma which is resistant to treatment with a PI3K delta inhibitor, more preferably the resistant haematological malignancy is idelasib-resistant diffuse large B-cell lymphoma, most preferably the resistant haematological malignancy is idelasib-resistant diffuse large B-cell lymphoma and the Gadd45β/MKK7 inhibitor is DTP3. In one preferred embodiment the resistant haematological malignancy is diffuse large B-cell lymphoma which is resistant to treatment with a Bcl-2 inhibitor, more preferably the resistant haematological malignancy is ABT-199-resistant diffuse large B-cell lymphoma, most preferably the resistant haematological malignancy is ABT-199-resistant diffuse large B-cell lymphoma and the Gadd45β/MKK7 inhibitor is DTP3. In one preferred embodiment the resistant haematological malignancy is diffuse large B-cell lymphoma which is resistant to treatment with a combination of i) cyclophosphamide, ii) doxorubicin, iii) vincristine, and iv) prednisone. In one preferred embodiment the resistant haematological malignancy is diffuse large B-cell lymphoma which is resistant to treatment with a combination of i) cyclophosphamide, ii) doxorubicin, iii) vincristine, and iv) prednisone, and the Gadd45β/MKK7 inhibitor is DTP3. In one preferred embodiment the resistant haematological malignancy is diffuse large B-cell lymphoma which is resistant to treatment with a combination of i) cyclophosphamide, ii) doxorubicin, iii) vincristine, iv) prednisone, and v) rituximab. In one preferred embodiment the resistant haematological malignancy is diffuse large B-cell lymphoma which is resistant to treatment with a combination of i) cyclophosphamide, ii) doxorubicin, iii) vincristine, iv) prednisone, and v) rituximab, and the Gadd45β/MKK7 inhibitor is DTP3. In one preferred embodiment the resistant haematological malignancy is diffuse large B-cell lymphoma which is resistant to treatment with a combination of i) fludaribine, and ii) cyclophosphamide. In one preferred embodiment the resistant haematological malignancy is diffuse large B-cell lymphoma which is resistant to treatment with a combination of i) fludaribine, and ii) cyclophosphamide, and the Gadd45β/MKK7 inhibitor is DTP3. In one preferred embodiment the resistant haematological malignancy is diffuse large B-cell lymphoma which is resistant to treatment with a combination of i) fludaribine, and ii) rituximab. In one preferred embodiment the resistant haematological malignancy is diffuse large B-cell lymphoma which is resistant to treatment with a combination of i) fludaribine, and ii) rituximab, and the Gadd45β/MKK7 inhibitor is DTP3. In one preferred embodiment the resistant haematological malignancy is diffuse large B-cell lymphoma which is resistant to treatment with a combination of i) fludaribine, ii) cyclophosphamide, and iii) rituximab. In one preferred embodiment the resistant haematological malignancy is diffuse large B-cell lymphoma which is resistant to treatment with a combination of i) fludaribine, ii) cyclophosphamide, and iii) rituximab, and the Gadd45β/MKK7 inhibitor is DTP3.

In one preferred embodiment, the resistant haematological malignancy is mucosa-associated lymphoid tissue (MALT) lymphoma which is resistant to treatment with an anti-cancer agent selected from the group consisting of an anti-CD20 agent (e.g. rituximab), a cytotoxic agent (e.g. cyclophosphamide), an anthracycline antibiotic anti-cancer agent (e.g. doxorubicin), a mitotic inhibitor (e.g. vincristine), a glucocorticoid (e.g. prednisolone, dexamethasone), a purine analogue (e.g. fludarabine) and an anthracenedione antineoplastic agent (e.g. mitoxantrone). In one preferred embodiment the resistant haematological malignancy is MALT lymphoma which is resistant to treatment with an anti-CD20 agent, more preferably the resistant haematological malignancy is rituximab-resistant MALT lymphoma. In one preferred embodiment the resistant haematological malignancy is MALT lymphoma which is resistant to treatment with a cytotoxic agent, more preferably the resistant haematological malignancy is cyclophosphamide-resistant MALT lymphoma. In one preferred embodiment the resistant haematological malignancy is MALT lymphoma which is resistant to treatment with an anthracycline antibiotic anti-cancer agent, more preferably the resistant haematological malignancy is doxorubicin-resistant MALT lymphoma. In one preferred embodiment the resistant haematological malignancy is MALT lymphoma which is resistant to treatment with a mitotic inhibitor, more preferably the resistant haematological malignancy is vincristine-resistant MALT lymphoma. In one preferred embodiment the resistant haematological malignancy is MALT lymphoma which is resistant to treatment with a glucocorticoid, more preferably the resistant haematological malignancy is prednisolone-resistant MALT lymphoma or dexamethasone-resistant MALT lymphoma. In one preferred embodiment the resistant haematological malignancy is MALT lymphoma which is resistant to treatment with a purine analogue, more preferably the resistant haematological malignancy is fludarabine-resistant MALT lymphoma. In one preferred embodiment the resistant haematological malignancy is MALT lymphoma which is resistant to treatment with an anthracenedione antineoplastic agent, more preferably the resistant haematological malignancy is mitoxantrone-resistant MALT lymphoma. In one preferred embodiment the resistant haematological malignancy is MALT lymphoma which is resistant to treatment with a combination of i) cyclophosphamide, ii) doxorubicin, iii) vincristine, and iv) prednisolone, prednisone or dexamethasone. In one preferred embodiment the resistant haematological malignancy is MALT lymphoma which is resistant to treatment with a combination of i) cyclophosphamide, ii) doxorubicin, iii) vincristine, and iv) prednisolone, prednisone or dexamethasone, and the Gadd45β/MKK7 inhibitor is DTP3. In one preferred embodiment the resistant haematological malignancy is MALT lymphoma which is resistant to treatment with a combination of i) cyclophosphamide, ii) doxorubicin, iii) vincristine, iv) prednisone, prednisolone or dexamethasone, and v) rituximab. In one preferred embodiment the resistant haematological malignancy is MALT lymphoma which is resistant to treatment with a combination of i) cyclophosphamide, ii) doxorubicin, iii) vincristine, iv) prednisone, prednisolone or dexamethasone, and v) rituximab, and the Gadd45β/MKK7 inhibitor is DTP3. In one preferred embodiment the resistant haematological malignancy is MALT lymphoma which is resistant to treatment with a combination of i) fludaribine, ii) doxorubicin, and iii) dexamethasone, prednisone or prednisolone. In one preferred embodiment the resistant haematological malignancy is MALT lymphoma which is resistant to treatment with a combination of i) fludaribine, ii) doxorubicin, and iii) dexamethasone, prednisone or prednisolone, and the Gadd45β/MKK7 inhibitor is DTP3. In one preferred embodiment the resistant haematological malignancy is MALT lymphoma which is resistant to treatment with a combination of i) fludaribine, ii) doxorubicin, iii) dexamethasone, prednisone or prednisolone, and iv) rituximab. In one preferred embodiment the resistant haematological malignancy is MALT lymphoma which is resistant to treatment with a combination of i) fludaribine, ii) doxorubicin, iii) dexamethasone, prednisone or prednisolone, and iv) rituximab, and the Gadd45β/MKK7 inhibitor is DTP3. In one preferred embodiment the resistant haematological malignancy is MALT lymphoma which is resistant to treatment with a combination of i) fludaribine, ii) mitoxantrone, and iii) dexamethasone, prednisone or prednisolone. In one preferred embodiment the resistant haematological malignancy is MALT lymphoma which is resistant to treatment with a combination of i) fludaribine, ii) mitoxantrone, and iii) dexamethasone, prednisone or prednisolone, and the Gadd45β/MKK7 inhibitor is DTP3. In one preferred embodiment the resistant haematological malignancy is MALT lymphoma which is resistant to treatment with a combination of i) fludaribine, ii) mitoxantrone, iii) dexamethasone, prednisone or prednisolone, and iv) rituximab. In one preferred embodiment the resistant haematological malignancy is MALT lymphoma which is resistant to treatment with a combination of i) fludaribine, ii) mitoxantrone, iii) dexamethasone, prednisone or prednisolone, and iv) rituximab, and the Gadd45β/MKK7 inhibitor is DTP3. In one preferred embodiment the resistant haematological malignancy is MALT lymphoma which is resistant to treatment with a combination of i) cyclophosphamide, ii) vincristine, iii) dexamethasone, prednisone or prednisolone, and iv) rituximab. In one preferred embodiment the resistant haematological malignancy is MALT lymphoma which is resistant to treatment with a combination of i) cyclophosphamide, ii) vincristine, iii) dexamethasone, prednisone or prednisolone, and iv) rituximab, and the Gadd45β/MKK7 inhibitor is DTP3.

In one preferred embodiment, the resistant haematological malignancy is Hodgkin's lymphoma which is resistant to treatment with an anti-cancer agent selected from the group consisting of an anti-CD20 agent (e.g. rituximab), a cytotoxic agent (e.g. cyclophosphamide), an anthracycline antibiotic anti-cancer agent (e.g. doxorubicin), a mitotic inhibitor (e.g. vincristine), a glucocorticoid (e.g. prednisolone, dexamethasone), a purine analogue (e.g. fludarabine) and an anthracenedione antineoplastic agent (e.g. mitoxantrone). In one preferred embodiment the resistant haematological malignancy is Hodgkin's lymphoma which is resistant to treatment with an anti-CD20 agent, more preferably the resistant haematological malignancy is rituximab-resistant Hodgkin's lymphoma. In one preferred embodiment the resistant haematological malignancy is Hodgkin's lymphoma which is resistant to treatment with a cytotoxic agent, more preferably the resistant haematological malignancy is cyclophosphamide-resistant Hodgkin's lymphoma. In one preferred embodiment the resistant haematological malignancy is Hodgkin's lymphoma which is resistant to treatment with an anthracycline antibiotic anti-cancer agent, more preferably the resistant haematological malignancy is doxorubicin-resistant Hodgkin's lymphoma. In one preferred embodiment the resistant haematological malignancy is Hodgkin's lymphoma which is resistant to treatment with a mitotic inhibitor, more preferably the resistant haematological malignancy is vincristine-resistant Hodgkin's lymphoma. In one preferred embodiment the resistant haematological malignancy is Hodgkin's lymphoma which is resistant to treatment with a glucocorticoid, more preferably the resistant haematological malignancy is prednisolone-resistant Hodgkin's lymphoma or dexamethasone-resistant Hodgkin's lymphoma. In one preferred embodiment the resistant haematological malignancy is Hodgkin's lymphoma which is resistant to treatment with a purine analogue, more preferably the resistant haematological malignancy is fludarabine-resistant Hodgkin's lymphoma. In one preferred embodiment the resistant haematological malignancy is Hodgkin's lymphoma which is resistant to treatment with an anthracenedione antineoplastic agent, more preferably the resistant haematological malignancy is mitoxantrone-resistant Hodgkin's lymphoma. In one preferred embodiment the resistant haematological malignancy is Hodgkin's lymphoma which is resistant to treatment with a combination of i) cyclophosphamide, ii) doxorubicin, iii) vincristine, and iv) prednisolone, prednisone or dexamethasone. In one preferred embodiment the resistant haematological malignancy is Hodgkin's lymphoma which is resistant to treatment with a combination of i) cyclophosphamide, ii) doxorubicin, iii) vincristine, and iv) prednisolone, prednisone or dexamethasone, and the Gadd45β/MKK7 inhibitor is DTP3. In one preferred embodiment the resistant haematological malignancy is Hodgkin's lymphoma which is resistant to treatment with a combination of i) cyclophosphamide, ii) doxorubicin, iii) vincristine, iv) prednisone, prednisolone or dexamethasone, and v) rituximab. In one preferred embodiment the resistant haematological malignancy is Hodgkin's lymphoma which is resistant to treatment with a combination of i) cyclophosphamide, ii) doxorubicin, iii) vincristine, iv) prednisone, prednisolone or dexamethasone, and v) rituximab, and the Gadd45β/MKK7 inhibitor is DTP3. In one preferred embodiment the resistant haematological malignancy is Hodgkin's lymphoma which is resistant to treatment with a combination of i) fludaribine, ii) doxorubicin, and iii) dexamethasone, prednisone or prednisolone. In one preferred embodiment the resistant haematological malignancy is Hodgkin's lymphoma which is resistant to treatment with a combination of i) fludaribine, ii) doxorubicin, and iii) dexamethasone, prednisone or prednisolone, and the Gadd45β/MKK7 inhibitor is DTP3. In one preferred embodiment the resistant haematological malignancy is Hodgkin's lymphoma which is resistant to treatment with a combination of i) fludaribine, ii) doxorubicin, iii) dexamethasone, prednisone or prednisolone, and iv) rituximab. In one preferred embodiment the resistant haematological malignancy is Hodgkin's lymphoma which is resistant to treatment with a combination of i) fludaribine, ii) doxorubicin, iii) dexamethasone, prednisone or prednisolone, and iv) rituximab, and the Gadd45β/MKK7 inhibitor is DTP3. In one preferred embodiment the resistant haematological malignancy is Hodgkin's lymphoma which is resistant to treatment with a combination of i) fludaribine, ii) mitoxantrone, and iii) dexamethasone, prednisone or prednisolone. In one preferred embodiment the resistant haematological malignancy is Hodgkin's lymphoma which is resistant to treatment with a combination of i) fludaribine, ii) mitoxantrone, and iii) dexamethasone, prednisone or prednisolone, and the Gadd45β/MKK7 inhibitor is DTP3. In one preferred embodiment the resistant haematological malignancy is Hodgkin's lymphoma which is resistant to treatment with a combination of i) fludaribine, ii) mitoxantrone, iii) dexamethasone, prednisone or prednisolone, and iv) rituximab. In one preferred embodiment the resistant haematological malignancy is Hodgkin's lymphoma which is resistant to treatment with a combination of i) fludaribine, ii) mitoxantrone, iii) dexamethasone, prednisone or prednisolone, and iv) rituximab, and the Gadd45β/MKK7 inhibitor is DTP3. In one preferred embodiment the resistant haematological malignancy is Hodgkin's lymphoma which is resistant to treatment with a combination of i) cyclophosphamide, ii) vincristine, iii) dexamethasone, prednisone or prednisolone, and iv) rituximab. In one preferred embodiment the resistant haematological malignancy is Hodgkin's lymphoma which is resistant to treatment with a combination of i) cyclophosphamide, ii) vincristine, iii) dexamethasone, prednisone or prednisolone, and iv) rituximab, and the Gadd45β/MKK7 inhibitor is DTP3.

A skilled medical practitioner is able to determine whether a patient having a haematological malignancy is responsive to treatment with an anti-cancer agent, or whether the haematological malignancy is resistant to treatment with the anti-cancer agent, using routine techniques. For example, a biological sample obtained from a subject (e.g. a biopsy or sample of blood, spleen, lymph node or bone marrow) may be tested for the presence and/or level of a biomarker associated with resistance of the haematological malignancy. The level of a biomarker in a biological sample obtained from a subject having a suspected resistant haematological malignancy may for example be used to determine a probability that said subject has said resistant haematological malignancy, e.g. by using a statistical model constructed based on the levels of said biomarker present in a patient population having the resistant haematological malignancy and based on the levels of said biomarker present in a patient population having said haematological malignancy which is not resistant. In some embodiments, a subject is determined as having a resistant haematological malignancy if the probability that the subject has said resistant haematological malignancy is at least 55%, at least 60%, at least 65%, at least 70%, at least 75%, at least 80%, at least 85%, at least 90% or at least 95%.

Alternatively, a skilled medical practitioner is able to determine whether a patient having a haematological malignancy (for example, multiple myeloma) is responsive to treatment with an anti-cancer agent, or whether the haematological malignancy is resistant to treatment with the anti-cancer agent, by reviewing the clinical course of the disease and/or the subject's prognosis. For example, a patient may be determined to have a haematological malignancy which is resistant to treatment with the anti-cancer agent, if the patient has been treated with the anti-cancer agent yet shows less clinical improvement than would be expected in an equivalent patient who was responsive to treatment with the anti-cancer agent. Because some types of resistance typically develop by a process of selection in response to earlier exposure to the anti-cancer treatment, in some embodiments of the invention it may be appropriate to determine that a patient is resistant to treatment with an anti-cancer agent if that patient initially showed clinical improvement with that agent, but no longer does so. In other patients, resistance may be intrinsic rather than acquired, and in those patients resistance will be present before the patient is exposed to the agent. Clinical improvement, or the lack thereof, may be assessed using any appropriate clinical parameter. For example, “tumour load” may be used and may be obtained by calculating an estimated number of tumour cells in the body using biopsy cell count data.

In the case of multiple myeloma, in some embodiments a subject may be determined to have resistant multiple myeloma if they are classified as having relapse, refractory or high risk multiple myeloma. In some embodiments, a subject may be determined to have resistant multiple myeloma if they are classified as having relapse or refractory multiple myeloma. In some embodiments, a subject may be determined to have resistant multiple myeloma if they are classified as having refractory multiple myeloma.

The type of multiple myeloma may for example be determined using the International Staging System, which stages the myeloma based on two factors: amount of beta-2-microglobulin in the blood and the level of serum albumin in the blood (Greipp et al, Journal of Clinical Oncology, 2005, 23 (15), 3412-3420):

Stage I: Serum β2 microglobulin <3.5 mg/L; Serum albumin ≧3.5 g/dL
Stage II: Not I or III* Either Serum β2 microglobulin <3.5 mg/L; but Serum albumin <3.5 g/dL; or Either Serum β2 microglobulin 3.5-5.5 mg/L irrespective of serum albumin.
Stage III: Serum β2 microglobulin >5.5 mg/L

The staging of multiple myeloma may be used in order to determine if a subject has resistant multiple myeloma. For example, a patient who rapidly progresses through the stages when being treated with an anti-cancer agent, may be determined, in some embodiments, to have resistant multiple myeloma. Accordingly, in one embodiment a subject is determined as having resistant multiple myeloma if, following administration of a course of an anti-cancer agent indicated for multiple myeloma, the subject progresses from Stage I to Stage II or III, or from Stage II to Stage III as defined by the International Staging System. In one embodiment a subject is determined as having resistant multiple myeloma if, whilst during administration of a course of an anti-cancer agent indicated for multiple myeloma, the subject progresses from Stage I to Stage II or III, or from Stage II to Stage III as defined by the International Staging System.

In the case of diffuse large B-cell lymphoma (DLBCL), DLBCL can be divided into prognostically significant subgroups with germinal centre B-cell-like (GCB), activated B-cell-like (ABC) or type 3 gene expression profiles, using expression profiling techniques or by immunohistochemistry. DLBCL can also be classified into GC-group or non-GC group. (Mareschal et al, Haematologica, 2011; 96. (12): 1888-90; Hans et al, Blood, 2004; 103(1): 275-82; Alizadeh et al., Nature, 2000; 403(6769):503-11; Rosenwald et al, N. Engl. J. Med. 2002; 346(25): 1937-47).

In some embodiments, a subject is determined as having resistant DLBCL if they are classified as having activated B-cell-like (ABC) DLBCL. In some embodiments, a subject has resistant DLBL if they are classified as having GC-group DLBCL.

As mentioned above, the subject may be a subject who has already been administered a therapeutic agent other than a Gadd45β/MKK7 inhibitor, and has relapsed or is refractory. Accordingly, in some embodiments, prior to administration of the Gadd45β/MKK7 inhibitor to the subject, a course of a different therapeutic agent indicated for said haematological malignancy is administered to the subject. In some preferred embodiments, the Gadd45β/MKK7 inhibitor is for use in a method comprising: administering a therapeutic agent indicated for a haematological malignancy, determining whether the haematological malignancy is a haematological malignancy which is resistant to treatment with the therapeutic agent; and when the haematological malignancy is determined to be a haematological malignancy which is resistant to treatment with the anti-cancer agent, administering the Gadd45β/MKK7 inhibitor to the subject; or when the haematological malignancy is determined to be a haematological malignancy which is not resistant to treatment with the anti-cancer agent, not administering the Gadd45β/MKK7 inhibitor to the subject.

In other embodiments, the subject may not have been administered a course of a different therapeutic agent indicated for said haematological malignancy prior to administration of the Gadd45β/MKK7 inhibitor. For example, it may be that initial tests carried out on biological samples obtained from a subject lead to a determination that the haematological malignancy is resistant, in which case the first course of therapy will include administration of a Gadd45β/MKK7 inhibitor. In some preferred embodiments, the Gadd45β/MKK7 inhibitor is for use in a method comprising: analysing a biological sample obtained from a subject having a suspected resistant haematological malignancy and determining i) the level and/or ii) the presence or absence of a biomarker associated with the resistant haematological malignancy; determining whether the haematological malignancy is a resistant haematological malignancy; and when the haematological malignancy is determined to be a resistant haematological malignancy, administering the Gadd45β/MKK7 inhibitor to the subject; or when the haematological malignancy is determined to be a haematological malignancy which is not resistant, not administering the Gadd45β/MKK7 inhibitor to the subject.

In some embodiments of the invention, whether or not a haematological malignancy is resistant to treatment with an anti-cancer agent is determined by taking a biopsy sample of cells from the haematological malignancy and culturing them in vitro. The anti-cancer agent is then added to the culture at a clinically relevant concentration and the anti-cancer agent's effect on survival of the cells is assessed (for example based on the % of cells surviving at a given timepoint, or based on the IC50 value for the anti-cancer agent). If the cells are resistant in vitro, it may be determined that the haematological malignancy from which those cells derive is also resistant to the anti-cancer agent. Further guidance on suitable in vitro assays may be found elsewhere in this specification, for example, in the examples.

In some embodiments, whether or not a haematological malignancy is resistant to treatment with an anti-cancer agent may be determined by gene sequencing using routine techniques (see e.g. Mardis and Wilson, Human Molecular Generics, 2009, Volume 18, Issue 2, R163-R168; Meldrum et al, Clin. Biochem. Rev. 2011, Vol 32, p 177-195), in order to identify whether a sample obtained from a subject having a haematological malignancy has a genome associated with a resistant form of said haematological malignancy.

In the case of multiple myeloma, resistant multiple myeloma may be associated with IgH translocation t (4:14)(p16.3; q32.3). Other biomarkers associated with resistant multiple myeloma include increased expression of Bcl2, increased expression of inhibitors of apoptosis protein; increased expression of multidrug resistance gene (MDR); presence of growth-promoting cytokines within the bone marrow microenvironment such as IL-6 and IGF-1. Further biomarkers associated with resistant multiple myeloma include mutations of genes encoding for subunits of the proteasome or the IMiD therapeutic target, cerebron (CRBM). In addition, resistant multiple myeloma may be associated with mutations in genes encoding factors within the NF-κB pathway such as CARD11/CARMA1, TNFAIP3/A20, CD79A, CD79B and/or MYD88. A common mechanism of resistance in cancer involves downregulation of cell surface receptors or proteins, and so other biomarkers associated with resistant multiple myeloma may include downregulation of CD38 and/or SLAM7.

In the case of diffuse large B-cell lymphoma, resistant DLBCL may be associated with mutations in genes encoding factors within the NF-κB pathway such as CARD11/CARMA1, TNFAIP3/A20, CD79A, CD79B and/or MYD88 (e.g. L265P), as well as mutations in Bcl2 (e.g. Bcl2 translocation t(14:18) (q32:q21)), TAK1. Other biomarkers associated with resistant DLBCL include increased expression of XIAP (X-linked inhibitor of apoptosis), increased expression of 14-3-3ζ and amplification of c-Rel As mentioned above, downregulation of cell surface receptors common mechanism of resistance in cancer involves downregulation of cell surface receptors or proteins, and so other biomarkers associated with resistant DLBCL may include downregulation of CD20.

Resistant MALT lymphoma may be associated with mutations in genes encoding factors within the NF-κB pathway such as CARD11/CARMA1, TNFAIP3/A20, CD79A, CD79B and/or MYD88 In addition, downregulation of CD20 may be associated with having MALT lymphoma.

The invention also provides use of a Gadd45β/MKK7 inhibitor for the manufacture of a medicament for the treatment of a resistant haematological malignancy in a subject.

Preferences in relation to such uses (e.g. relating to the Gadd45β/MKK7 inhibitor, the haematological malignancy) are the same as those preferences indicated above in relation to a Gadd45β/MKK7 inhibitor for use in a method of treating a resistant haematological malignancy in a subject, the method comprising the step of administering the Gadd45β/MKK7 inhibitor to the subject.

The invention also provides a method of treating a resistant haematological malignancy in a subject, the method comprising the step of administering a therapeutically effective amount of a Gadd45β/MKK7 inhibitor to the subject. Preferences in relation to such methods (e.g. relating to the Gadd45β/MKK7 inhibitor, the haematological malignancy) are the same as those preferences indicated above in relation to a Gadd45β/MKK7 inhibitor for use in a method of treating a resistant haematological malignancy in a subject, the method comprising the step of administering the Gadd45β/MKK7 inhibitor to the subject.

### Combination Therapies

As discussed above, the Gadd45β/MKK7 inhibitor DTP3 has been found to retain full therapeutic efficacy against a range of multiple myeloma cell lines, including those which are resistant to current multiple myeloma treatments such as bortezomib, dexamethasone and lenalidomide. These results suggest that therapies based on the combination of Gadd45β/MKK7 inhibitors such as DTP3, which operate by a different mechanism to known therapeutic agents useful for treating haematological malignancies, with other anti-cancer agents, will be particularly effective in treating haematological malignancies. Further, the inventors have found that the combination of DTP3 with bortezomib displayed synergistic activity in different multiple myeloma cell lines. In other words, use of a combination therapy including DTP3 as a component provides unexpectedly effective results.

Accordingly, the present invention also provides a Gadd45β/MKK7 inhibitor for use as described herein, wherein the Gadd45β/MKK7 inhibitor administered in combination with another anti-cancer agent. The other anti-cancer agent may, for example, be one or more of an agent selected from the group consisting of a proteasome inhibitor (e.g. bortezomib, carfilzomib), an IMiD anti-cancer agent (e.g. lenalidomide, thalidomide, pomalidomide), a glucocorticoid (e.g. dexamethasone, prednisolone, prednisone), an anti-CD38 agent (e.g. daratumamab, for example an anti-CD 38 agent in combination with lenalidomide), an anti-SLAM7 agent (e.g. elotuzumab), a Bruton's tyrosine kinase inhibitor (e.g. ibrutinib), a protein kinase C inhibitor (e.g. sotrastaurin), an anti-CD20 agent (e.g. rituximab), a cytotoxic agent (e.g. cyclophosphamide), an alkylating agent (e.g. melphalan), an anthracycline antibiotic anti-cancer agent (e.g. doxorubicin), a mitotic inhibitor (e.g. vincristine), a purine analogue (e.g. fludarabine), a PI3Kdelta inhibitor (e.g. idelasib), a Bcl-2 inhibitor (e.g. ABT-199) and histone deacetylase (HDAC) inhibitor (eg Panobinostat). The other anti-cancer agent may, for example, be a therapeutic agent approved for the treatment of a haematological malignancy. The other anti-cancer agent may, for example, be one or more of an agent selected from the group consisting of a therapeutic agent approved for the treatment of multiple myeloma, a therapeutic agent approved for the treatment of diffuse large B-cell lymphoma, a therapeutic agent approved for the treatment of mucosa-associated lymphoid tissue (MALT) lymphoma, a therapeutic agent approved for the treatment of Hodgkin's lymphoma, a therapeutic agent approved for the treatment of marginal zone lymphoma, a therapeutic agent approved for the treatment of chronic lymphocytic leukaemia or a therapeutic agent approved for the treatment of acute myelogenic leukaemia.

The present invention further provides a combination comprising i) DTP3, or a derivative or a salt thereof

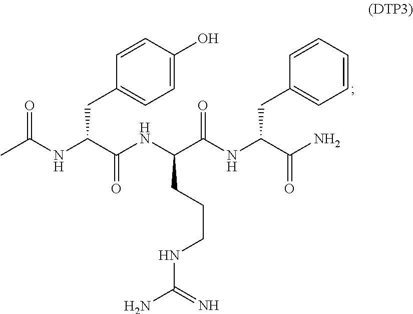 [SMILES: CC(=O)N[C@H](Cc1ccc(O)cc1)C(=O)N[C@H](CCCNC(=N)N)C(=O)N[C@H](Cc1ccccc1)C(N)=O]

and
ii) a further anti-cancer agent which is a proteasome inhibitor. Preferably the proteasome inhibitor is selected from the group consisting of bortezomib and carfilzomib, more preferably the proteasome inhibitor is bortezomib.

The invention also provides a combination comprising i) DTP3, or a salt thereof; and ii) a further anti-cancer agent which is an IMiD anti-cancer agent. Preferably, the IMiD anti-cancer agent is selected from the group consisting of lenalidomide, thalidomide and pomalidomide, more preferably the IMiD anti-cancer agent is lenalidomide.

The invention also provides a combination comprising i) DTP3, or a salt thereof; and ii) a further anti-cancer agent which is a glucocorticoid. Preferably the glucocorticoid is selected from the group consisting of dexamethasone, prednisolone and prednisone, more preferably the glucocorticoid is dexamethasone or prednisone. In one embodiment the glucocorticoid is dexamethasone. In another embodiment the glucocorticoid is prednisone.

The invention also provides a combination comprising i) DTP3, or a salt thereof; ii) bortezomib or cafilzomib; iii) dexamethasone, prednisone or prednisolone; and iv) lenalidomide or pomalidomide. The invention also provides a combination comprising i) DTP3, or a salt thereof; ii) bortezomib; iii) dexamethasone; and iv) lenalidomide.

The combinations of the invention listed above are useful as medicaments, e.g. for the treatment of haematological malignancies such as those listed above. The combinations find use in treating resistant and non-resistant forms of haematological malignancies. The hematological malignancy is preferably multiple myeloma.

The invention also provides DTP3 or a salt thereof for use in a method of treating a haematological malignancy, wherein DTP3 or a salt thereof is administered in combination with a proteasome inhibitor, and wherein DTP3 or a salt thereof increases the efficacy of said proteasome inhibitor. Preferably the proteasome inhibitor is bortezomib. Preferably the haematological malignancy is multiple myeloma.

The invention also provides DTP3 or a salt thereof for use in a method of treating a haematological malignancy, wherein DTP3 or a salt thereof is administered in combination with a glucocorticoid, and wherein DTP3 or a salt thereof increases the efficacy of said glucocorticoid. Preferably the glucocorticoid is dexamethasone. Preferably the haematological malignancy is multiple myeloma.

The invention also provides DTP3 or a salt thereof for use in a method of treating a haematological malignancy, wherein DTP3 or a salt thereof is administered in combination with an IMiD anti-cancer agent, and wherein DTP3 or a salt thereof increases the efficacy of said IMiD anti-cancer agent. Preferably the IMiD anti-cancer agent is lenalidomide.

The invention also provides DTP3 or a salt thereof for use in a method of treating a haematological malignancy, wherein DTP3 or a salt thereof is administered in combination with bortezomib, dexamethasone and lenalidomide, and wherein DTP3 or a salt thereof increases the efficacy of bortezomib, dexamethasone and lenalidomide.

The invention also provides methods of treating haematological malignancies in a subject, comprising administering to the subject a therapeutically effective amount of any one of the combinations described above.

The invention also provides use of any one of the combinations described above for the manufacture of a medicament for the treatment of a haematological malignancy.

Where a Gadd45β/MKK7 inhibitor is used in combination with a further therapeutic agent, the further therapeutic agent may be used, for example, in those amounts indicated in the Physicians' Desk Reference (PDR) or as otherwise determined by one of ordinary skill in the art.

Where the compound of the invention is administered in combination with a further therapeutic agent, the individual components of such a combination may be administered simultaneously, sequentially, or separately (e.g. at different times during the course of therapy). The present invention is therefore to be understood as embracing all such regimes of simultaneous or alternating treatment and the term “administering” is to be interpreted accordingly.

### Reduction of Side Effects

It has been found that Gadd45β/MKK7 inhibitors such as DTP3 are particularly selective for cancer cells. In addition, no side-effects were observed upon administration at the effective dose of the Gadd45β/MKK7 inhibitor DTP3 to mice in xenograft models. Thus combination therapies involving the use of Gadd45β/MKK7 inhibitors together with other established anti-cancer agents which are less selective, offer the prospect of using those other agents at lower doses, thus providing improved therapies with fewer side-effects.

Accordingly, the invention provides DTP3 or a salt thereof for use in a method of treating a haematological malignancy, wherein DTP3 or a salt thereof is administered in combination with a proteasome inhibitor, and wherein DTP3 or a salt thereof is for use in preventing and/or reducing one or more side-effects associated with administration of said proteasome inhibitor. Preferably the proteasome inhibitor is bortezomib.

The invention also provides DTP3 or a salt thereof for use in a method of treating a haematological malignancy, wherein DTP3 or a salt thereof is administered in combination with an IMiD anti-cancer agent, and wherein DTP3 or a salt thereof is for use in preventing and/or reducing one or more side-effects associated with administration of said IMiD anti-cancer agent. Preferably the IMiD anti-cancer agent is lenalidomide.

The invention also provides DTP3 or a salt thereof for use in a method of treating a haematological malignancy, wherein DTP3 or a salt thereof is administered in combination with a glucocorticoid, and wherein DTP3 or a salt thereof is for use in preventing and/or reducing one or more side-effects associated with administration of said glucocorticoid. Preferably the glucocorticoid is dexamethasone.

Preferably the haematological malignancy is multiple myeloma, DLBCL or MALT lymphoma; more preferably multiple myeloma or DLBCL; most preferably multiple myeloma.

Side-effects associated with administration of bortezumib include nausea, vomiting, constipation, diarrhoea, reduction of white blood cells (increased risk of infection), reduction of platelets (increased risk of bruising or bleeding), anaemia, dizziness and fatigue.

Side-effects associated with administration of lenalidomide include diarrhoea, constipation, nausea, vomiting, and fatigue.

Side-effects associated with administration of dexamethasone include acne, amenorrhoea, bone fractures, bruising, Cushing's syndrome, diabetes, nausea, osteoporosis, raised blood pressure and thromboembolism.

The invention also provides a method of treating a haematological malignancy in a subject, the method comprising administering a therapeutically effective amount of DTP3 or a salt thereof, wherein DTP3 or a salt thereof is administered in combination with a proteasome inhibitor, and wherein DTP3 or a salt thereof prevents and/or reduces one or more side-effects associated with administration of said proteasome inhibitor.

The invention also provides a method of treating a haematological malignancy in a subject, the method comprising administering a therapeutically effective amount of DTP3 or a salt thereof, wherein DTP3 or a salt thereof is administered in combination with an IMiD anti-cancer agent, and wherein DTP3 or a salt thereof prevents and/or reduces one or more side-effects associated with administration of said IMiD anti-cancer agent.

The invention also provides a method of treating a haematological malignancy in a subject, the method comprising administering a therapeutically effective amount of DTP3 or a salt thereof, wherein DTP3 or a salt thereof is administered in combination with a glucocorticoid, and wherein DTP3 or a salt thereof prevents and/or reduces one or more side-effects associated with administration of said glucocorticoid.

The invention also provides use of DTP3 or a salt thereof for the manufacture of a medicament for the treatment of a haematological disorder, wherein DTP3 or a salt thereof is administered in combination with a proteasome inhibitor, and wherein DTP3 or a salt thereof prevents and/or reduces one or more side-effects associated with administration of said proteasome inhibitor.

The invention also provides use of DTP3 or a salt thereof for the manufacture of a medicament for the treatment of a haematological disorder, wherein DTP3 or a salt thereof is administered in combination with an IMiD anti-cancer agent, and wherein DTP3 or a salt thereof prevents and/or reduces one or more side-effects associated with administration of said IMiD anti-cancer agent.

The invention also provides use of DTP3 or a salt thereof for the manufacture of a medicament for the treatment of a haematological disorder, wherein DTP3 or a salt thereof is administered in combination with a glucocorticoid, and wherein DTP3 or a salt thereof prevents and/or reduces one or more side-effects associated with administration of said glucocorticoid.

### Pharmaceutical Compositions

The Gadd45β/MKK7 inhibitor used in the present invention is typically present in the form of a pharmaceutical composition comprising the Gadd45β/MKK7 inhibitor and a pharmaceutically acceptable carrier. Where the Gadd45β/MKK7 inhibitor is used in combination with one or more further therapeutic agents, the further therapeutic agent is also typically present in the form of a pharmaceutical composition comprising the further therapeutic agent and a pharmaceutically acceptable carrier. In one embodiment, the Gadd45β/MKK7 inhibitor and said further therapeutic agent are present in the same pharmaceutical composition. In another embodiment, the Gadd45β/MKK7 inhibitor and said further therapeutic agent are present in different pharmaceutical compositions.

Pharmaceutical compositions used in the present invention may take the form of a pharmaceutical formulation as described below.

The pharmaceutical formulations according to the invention include those suitable for oral, parenteral (including subcutaneous, intradermal, intramuscular, intravenous, and intraarticular), inhalation (including fine particle dusts or mists which may be generated by means of various types of metered dose pressurized aerosols, nebulizers or insufflators), rectal and topical (including dermal, transdermal, transmucosal, buccal, sublingual, and intraocular) administration, although the most suitable route may depend upon, for example, the condition and disorder of the recipient.

The formulations may conveniently be presented in unit dosage form and may be prepared by any of the methods well known in the art of pharmacy. All methods include the step of bringing an active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2S, 1988.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The active ingredient can, for example, be administered in a form suitable for immediate release or extended release. Immediate release or extended release can be achieved by the use of suitable pharmaceutical compositions comprising the active ingredient, or, particularly in the case of extended release, by the use of devices such as subcutaneous implants or osmotic pumps. The active ingredient can also be administered liposomally.

Preferably, compositions according to the invention are suitable for subcutaneous administration, for example by injection.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate and/or lactose and/or other excipients, binders, extenders, disintegrants, diluents and lubricants such as those known in the art. The active ingredient can also be delivered through the oral cavity by sublingual and/or buccal administration. Molded tablets, compressed tablets or freeze-dried tablets are exemplary forms which may be used. Exemplary compositions include those formulating the active ingredient with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor. An aqueous carrier may be, for example, an isotonic buffer solution at a pH of from about 3.0 to about 8.0, preferably at a pH of from about 3.5 to about 7.4, for example from 3.5 to 6.0, for example from 3.5 to about 5.0. Useful buffers include sodium citrate-citric acid and sodium phosphate-phosphoric acid, and sodium acetate/acetic acid buffers. The composition preferably does not include oxidizing agents and other compounds that are known to be deleterious to the compound of formula I and related molecules. Excipients that can be included are, for instance, other proteins, such as human serum albumin or plasma preparations. If desired, the pharmaceutical composition may also contain minor amounts of non-toxic auxiliary substances, such as wetting or emulsifying agents, preservatives, and pH buffering agents and the like, for example sodium acetate or sorbitan monolaurate.

Exemplary compositions for nasal aerosol or inhalation administration include solutions in saline, which can contain, for example, benzyl alcohol or other suitable preservatives, absorption promoters to enhance bioavailability, and/or other solubilizing or dispersing agents such as those known in the art. Conveniently in compositions for nasal aerosol or inhalation administration the compound of the invention is delivered in the form of an aerosol spray presentation from a pressurized pack or a nebulizer, with the use of a suitable propellant, e.g., dichlorodifluoro-methane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated to contain a powder mix of the compound and a suitable powder base, for example lactose or starch. In one specific, non-limiting example, a compound of the invention is administered as an aerosol from a metered dose valve, through an aerosol adapter also known as an actuator. Optionally, a stabilizer is also included, and/or porous particles for deep lung delivery are included (e.g., see U.S. Pat. No. 6,447,743).

Formulations for rectal administration may be presented as a retention enema or a suppository with the usual carriers such as cocoa butter, synthetic glyceride esters or polyethylene glycol. Such carriers are typically solid at ordinary temperatures, but liquefy and/or dissolve in the rectal cavity to release the drug.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase (mineral oil gelled with polyethylene).

Preferred unit dosage formulations are those containing an effective dose, as hereinbefore recited, or an appropriate fraction thereof, of active ingredient.

It should be understood that in addition to the ingredients particularly mentioned above, the formulations of this invention may include other agents conventional in the art having regard to the type of formulation in question, for example those suitable for oral administration may include flavouring agents.

The active ingredient is also suitably administered as a sustained-release system. Suitable examples of sustained-release systems of the invention include suitable polymeric materials, for example semi-permeable polymer matrices in the form of shaped articles, e.g., films, or microcapsules; suitable hydrophobic materials, for example as an emulsion in an acceptable oil; or ion exchange resins; and sparingly soluble derivatives of the active ingredient, for example, a sparingly soluble salt. Sustained-release systems may be administered orally; rectally; parenterally; intravaginally; intraperitoneally; topically, for example as a powder, ointment, gel, drop or transdermal patch; bucally; or as an oral or nasal spray.

Preparations for administration can be suitably formulated to give controlled release of active ingredient. For example, the pharmaceutical compositions may be in the form of particles comprising one or more of biodegradable polymers, polysaccharide jellifying and/or bioadhesive polymers, amphiphilic polymers, agents capable of modifying the interface properties of the particles of the active ingredient. These compositions exhibit certain biocompatibility features which allow a controlled release of the active substance. See U.S. Pat. No. 5,700,486.

Active ingredient may be delivered by way of a pump (see Langer, supra; Sefton, CRC Crit. Ref Biomed. Eng. 14:201, 1987; Buchwald et al., Surgery 88:507, 1980; Saudek et al., N. Engl. J. Med. 321:574, 1989) or by a continuous subcutaneous infusions, for example, using a mini-pump. An intravenous bag solution may also be employed. Other controlled release systems are discussed in the review by Langer (Science 249:1527-1533, 1990). In another aspect of the disclosure, active ingredient is delivered by way of an implanted pump, described, for example, in U.S. Pat. No. 6,436,091; U.S. Pat. No. 5,939,380; U.S. Pat. No. 5,993,414.

Implantable drug infusion devices are used to provide patients with a constant and long term dosage or infusion of active ingredient. Essentially such device may be categorized as either active or passive. An active ingredient may be formulated as a depot preparation. Such a long acting depot formulation can be administered by implantation, for example subcutaneously or intramuscularly; or by intramuscular injection. Thus, for example, active ingredient can be formulated with suitable polymeric or hydrophobic materials, for example as an emulsion in an acceptable oil; or ion exchange resins; or as a sparingly soluble derivatives, for example, as a sparingly soluble salt.

A therapeutically effective amount of an active ingredient may be administered as a single pulse dose, as a bolus dose, or as pulse doses administered over time. Thus, in pulse doses, a bolus administration of an active ingredient is provided, followed by a time period wherein no dose of that active ingredient is administered to the subject, followed by a second bolus administration. In specific, non-limiting examples, pulse doses of active ingredient are administered during the course of a day, during the course of a week, or during the course of a month.

The therapeutically effective amount of a Gadd45β/MKK7 inhibitor will be dependent on the molecule utilized, the subject being treated, the severity and type of the affliction, and the manner and route of administration.

The invention is further illustrated by the following non-limiting examples.

### Example 1: Gadd45β/MKK7 Inhibitors have Potent and Cancer Selective Activity

The Gadd45β/MKK7 inhibitors z-DTP1 (a tetrapeptide having the sequence Tyr-Asp-His-Phe, with amino acids in the D-configuration, and conjugated to an NH₂ group at the C terminal and to a benzyloxycarbonyl group at the N terminal) and z-DTP2 (a tetrapeptide having the sequence Tyr-Glu-Arg-Phe, with amino acids in the D configuration, and conjugated to an NH₂ group at the C terminal and to a benzyloxycarbonyl group at the N terminal) were tested for their activity against multiple myeloma cell lines.

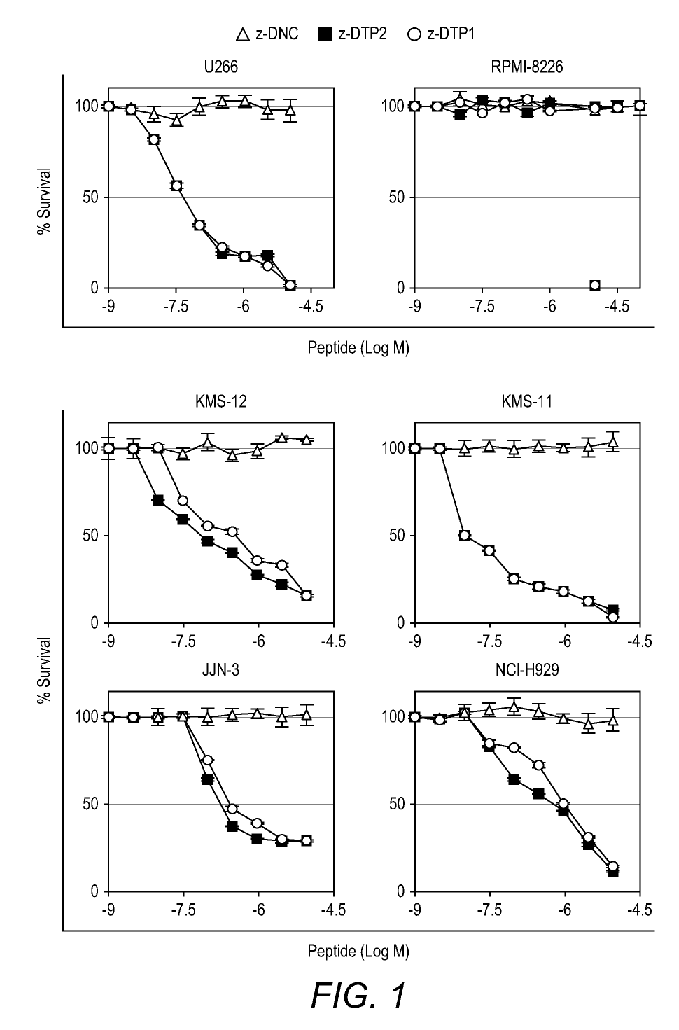

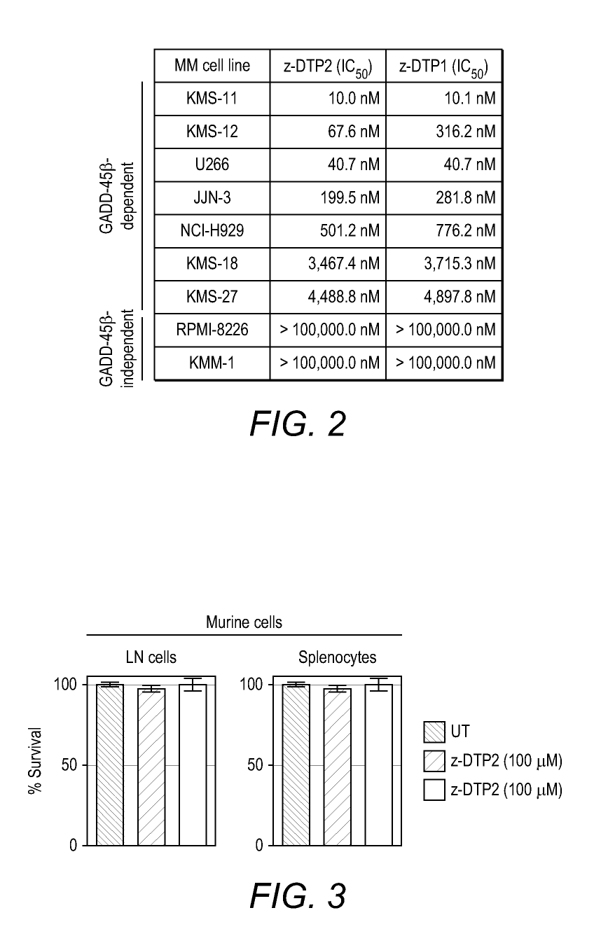

FIG. 1 shows the results of [³H]thymidine incorporation assays showing the survival of U266, KMS-12, KMS-11, JJN-3, NCI-H929 and RPMI-8226 multiple myeloma cell lines after a 6-day treatment with the indicated concentrations of z-DTP1, z-DTP2, or Z-protected (z)-DNC. FIG. 2 shows the IC₅₀ values of z-DTP1 and z-DTP2 at 144 hr, as determined by [³H]thymidine incorporation assays, in genetically heterogeneous multiple myeloma cell lines that either depend or do not depend on Gadd45β for survival.

As shown in FIGS. 1 and 2, the compounds exhibited potent cytotoxic activity across a panel of genetically heterogeneous multiple myeloma cell lines. Significantly, z-DTP1 and z-DTP2, but not the control D-tetrapeptide (z-DNC), induced potent and dose-dependent toxicity in all of the multiple myeloma cell lines tested, apart from the two which expressed nearly undetectable levels of GADD45β and low levels of MKK7 (i.e. the RPMI-8226 and KMM-1 cell lines)—exhibiting IC₅₀ values in the sensitive multiple myeloma cell lines in the low nM to low μM range.

FIG. 3 shows the survival of healthy mouse splenocytes and lymph node (LN) cells after treatment with z-DTP1 or z-DTP2 for 72 hr. Cell viability was measured using [³H]thymidine incorporation assays.

Importantly, both active D-peptides also exhibited an apparently complete lack of toxicity to normal cells, even when used at very high concentrations (i.e. 100 μM; FIG. 3). Hence, D-tetrapeptide antagonists of the Gadd45β/MKK7 complex show high activity and cancer-cell specificity in terms of apoptosis induction in multiple myeloma cells, without displaying any apparent toxicity to normal cells.

### Example 2: DTP3 is a Potent and Selective Gadd45β/MKK7 Inhibitor

The Gadd45β/MKK7 inhibitor DTP3 (a tripeptide having the sequence Tyr-Arg-Phe, with amino acids in the D-configuration, and conjugated to an NH₂ group at the C terminal and to an acetyl group at the N terminal) was tested for its activity against multiple myeloma cell lines.

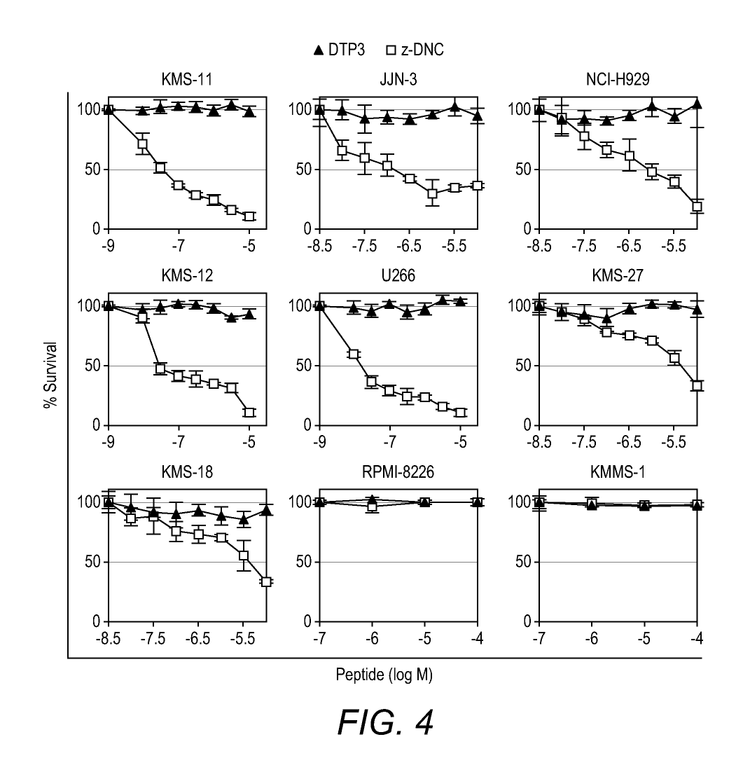

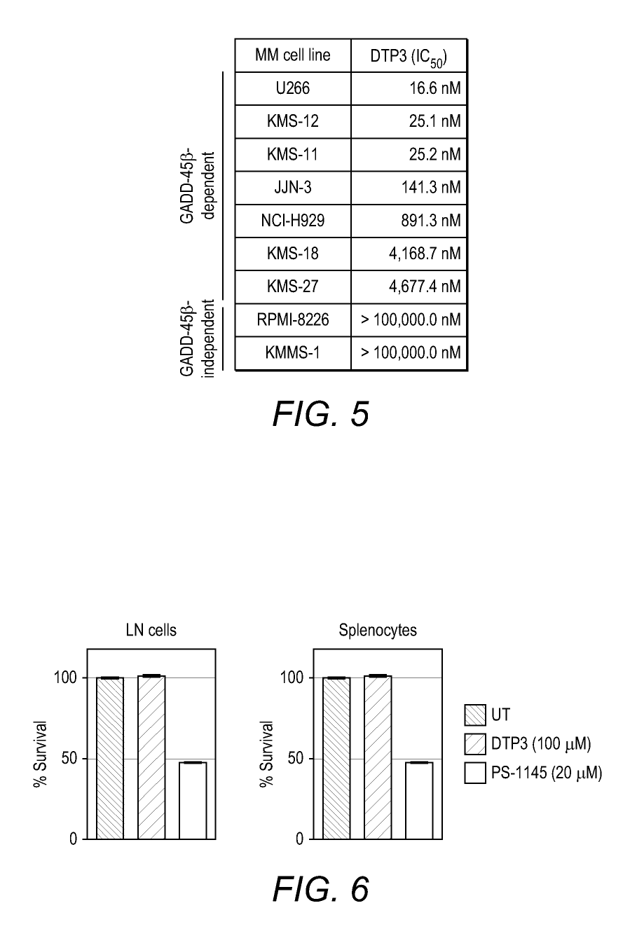

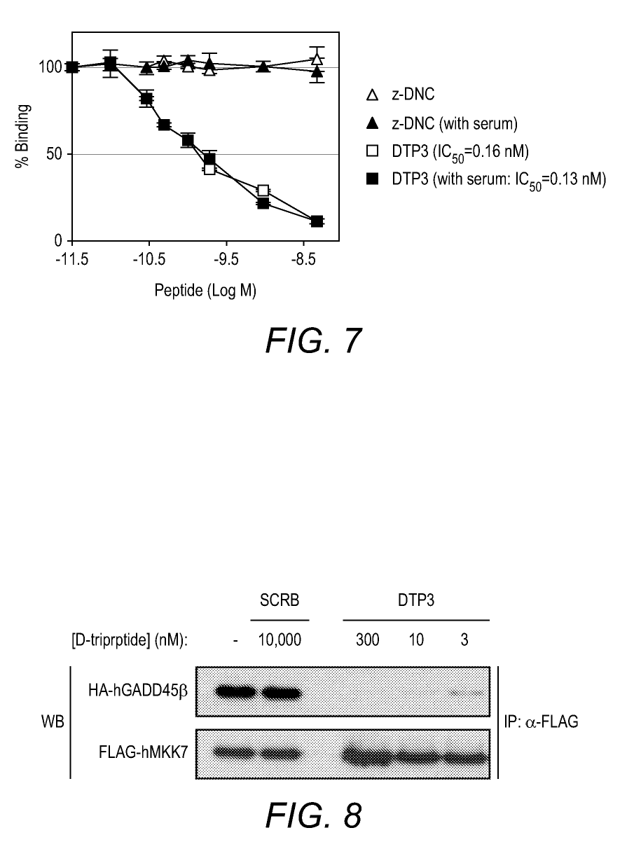

FIG. 4 shows the results of [³H]thymidine incorporation assays showing the survival of Gadd45β-dependent (top 2 rows and left of bottom row) and Gadd45β-independent (bottom row, middle and right) multiple myeloma cell lines after a 6-day treatment with the indicated concentrations of DTP3 or a negative control D-peptide (z-DNC). FIG. 5 shows the IC₅₀ values of DTP3 at 144 hr for the experiment shown in FIG. 4. FIG. 6 shows trypan blue exclusion assays showing the survival of mouse LN cells and splenocytes after treatment with DTP3 (100 μM) or PS-1145 (20 μM) for 144 hr. Values express the percentage of live cells present in the treated cultures relative to the live cells present in the respective untreated cultures and denote means±SD ([WM]; n=3). UT, untreated. FIG. 7 shows ELISA Gadd45β/MKK7 competition assays showing the IC₅₀ values of DTP3 and the scrambled control D-tripeptide, SCRB, before and after a 48-hr pre-incubation with human serum, at 37° C., as indicated. Values express the percentage of inhibition of Gadd45β binding to MKK7 relative to the binding measured in the absence of peptide. FIG. 8 shows co-immunoprecipitation assays showing the disruption of the Gadd45β/MKK7 complex by DTP3, but not by the SCRB control D-peptide, at the indicated concentrations. Co-immunoprecipitations (IP) were performed using an anti-FLAG antibody (a-FLAG); western blots (WB) were developed using an anti-HA or an anti-MKK7 antibody, as shown. The first column marked “-” shows incubation without D-tripeptide.

As shown in FIGS. 4 to 8, DTP3 is a potent and selective Gadd45β/MKK7 inhibitor, having sub-nM activity, high stability in vitro, and potent and selective capacity to kill multiple myeloma cells via apoptosis. DTP3 also displayed far lesser toxicity to normal cells than the IKKβ inhibitor PS-1145 (note the different concentrations of DTP3 and PS-1145 used).

### Example 3: The Gadd45β/MKK7 Inhibitor DTP3 is Effective in Killing Diffuse Large B-Cell Lymphoma Cell Lines Resistant to Current Therapies

Current treatment of diffuse large B-cell lymphoma typically involves treatment with combination therapy, such as cyclophosphamide, doxorubicin, vincristine and prednisone (CHOP), or rituximab, cyclophosphamide, doxorubicin, vincristine and prednisone (R-CHOP). With those current treatments, many patients relapse and/or develop drug resistance at some point. The potential of the Gadd45β/MKK7 inhibitor DTP3 to operate in these settings was evaluated, by determining levels of apoptosis in diffuse large B-cell lymphoma cell lines known to be resistant to current therapies (HT, SU-DHL-8, U-2932, RC-K8 and RIVA, see for example Kaneko et al, Clinical Cancer Research, 2014, 20, p 1814-1820; Naylor et al, Cancer Research, 2011, Volume 71, p 2643-2653; Lyu et al, Cancer Research, 2013, Volume 73, Issue 8, Supplement 1), following treatment of those cells with DTP3. These cells have CARD11, A20 and/or MYD88 mutations and are also known to be resistant to agents such as Bruton's tyrosine kinase inhibitors, spleen tyrosine kinase inhibitors, PI3K inhibitors and/or protein kinase C inhibitors.

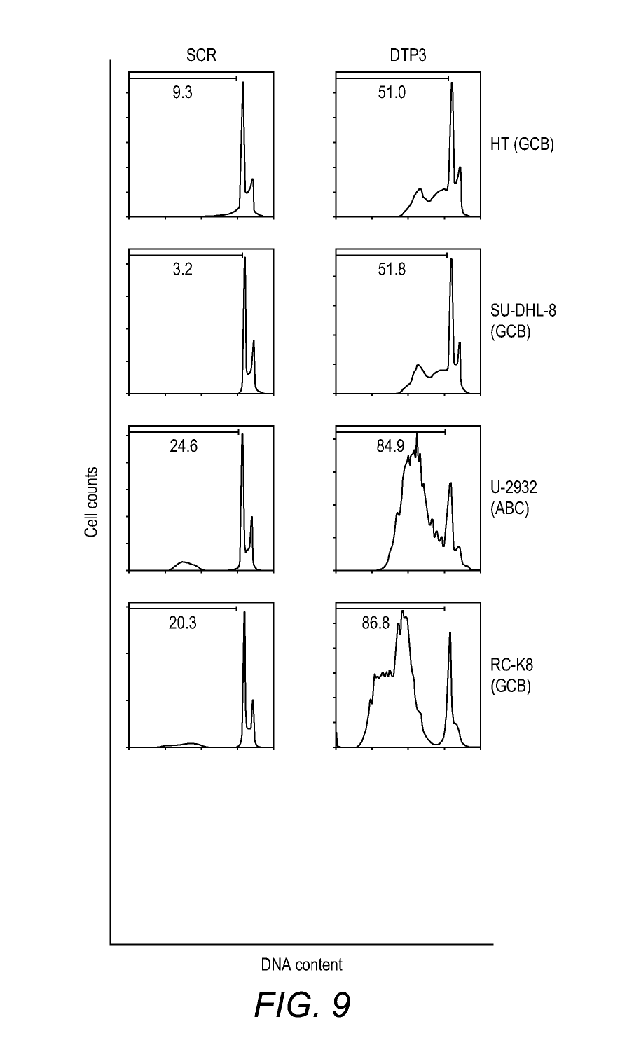

FIG. 9 shows PI nuclear staining assays showing apoptotic cells in diffuse large B-cell lymphoma (DLBCL) cell lines (HT, SU-DHL-8, U-2932 and RC-K8) following treatment with 10 μM of either DTP3 or the scrambled control D-tripeptide, SCR for 6 days. The percentages of apoptotic cells are depicted.

As shown in FIG. 9, DTP3 was effective in causing apoptosis in DLBCL cell lines that are resistant to conventional DLBCL treatments, such as rituximab, cyclophosphamide, doxorubicin, vincristine and prednisone, as well as to newer agents such as Bruton's tyrosine kinase inhibitors and protein kinase C inhibitors. These results provide strong evidence of the high therapeutic potential of DTP3 in DLBCL patients.

### Example 4: The Gadd45β/MKK7 Inhibitor DTP3 is Effective in Killing Multiple Myeloma Cell Lines Resistant to Current Therapies

With current standard multiple myeloma treatment, many patients will relapse and/or develop drug resistance at some point. The potential of the Gadd45β/MKK7 inhibitor DTP3 to operate in these settings was evaluated.

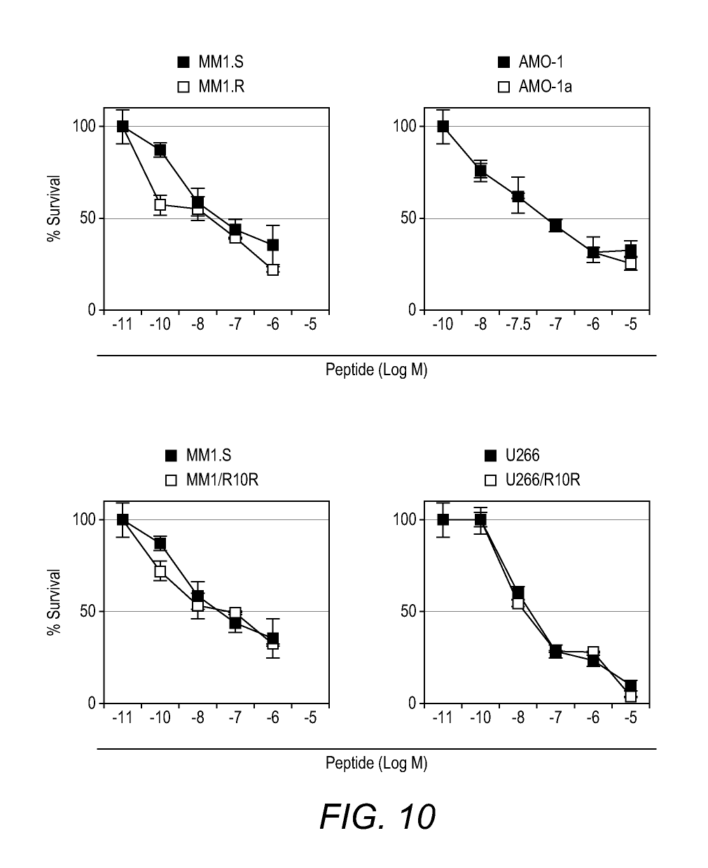

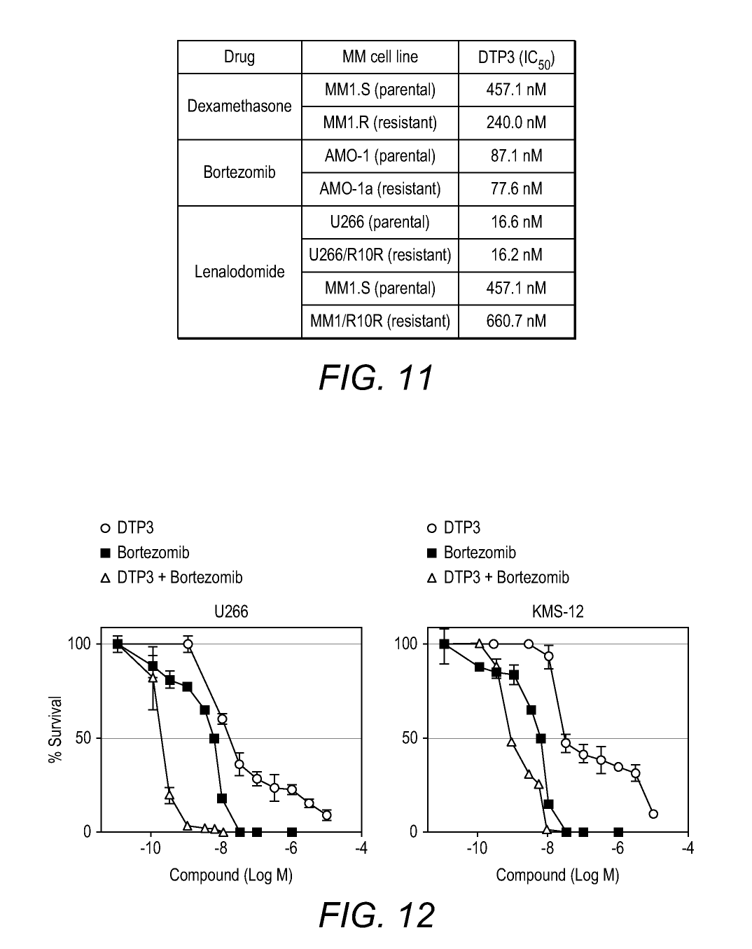

FIG. 10 shows the results of [³H]thymidine incorporation assays showing the survival of sensitive and drug-resistant multiple myeloma cell lines following a 6-day treatment with the indicated concentrations of DTP3. Values express the percentage of the cpm measured with the treated cultures relative to the cpm measured with the respective untreated cultures and denote means±SD (n=3). Matching pairs of sensitive (parental) and drug-resistant multiple myeloma cell lines were as follows: MM1.S (parental) and MM1.R (dexamethasone-resistant); AMO-1 (parental) and AMO-1a (bortezomib-resistant); MM1.S (parental) and MM1/R10R (lenalidomide-resistant); U266 (parental) and U266/R10R (lenalidomide-resistant). Data with the parental U266 MM multiple myeloma cell line were from the experiment shown in FIGS. 4 and 5 (5 out of 7 DTP3 concentrations only). For clarity purposes, the same data for the parental MM1.S MM multiple myeloma cell line are shown as control for the dexamethasone-resistant (MM1.R) MM multiple myeloma cell line and the lenalidomide-resistant (MM1/R10R) MM multiple myeloma cell line. FIG. 11 shows IC₅₀ values of DTP3 for the experiment shown in FIG. 10.

As shown in FIGS. 10 and 11, DTP3 retained full therapeutic efficacy in MM multiple myeloma cell lines that were resistant to conventional multiple myeloma treatments, such as dexamethasone, bortezomib and lenalidomide (Bjorklund et al., 2011; Bjorklund et al., 2014; Ruckrich et al., 2009). These results provide compelling evidence of the high therapeutic potential of DTP3 in multiple myeloma patients.

### Example 5: The Gadd45β/MKK7 Inhibitor DTP3 Synergises with the Proteasome Inhibitor Bortezomib

Current standard multiple myeloma treatment consists of combination therapy and, as discussed above, many patients will relapse and/or develop drug resistance to those therapies at some point. The effectiveness of the Gadd45β/MKK7 inhibitor DTP3 as a combination therapy with the proteasome inhibitor bortezomib was investigated.

FIG. 12 shows the results of [³H]thymidine incorporation assays showing the survival of representative GADD45β-dependent multiple myeloma cell lines after treatment with the indicated concentrations of DTP3 and bortezomib, used either as single agents or in combination. Treatments with DTP3 were for 6 days; bortezomib was added to the cell cultures 48 hr prior to the measurement of cell viability. For the combination treatment, DTP3 was used at the concentrations of 3 nM in U266 cells and of 10 nM in KMS-12 cells, whereas bortezomib was used at increasing concentrations, as shown. Based on the data shown, the IC₅₀ value of bortezomib as single agent at 48 hr was 6 nM in each of the two multiple myeloma cell lines. The survival curves of U266 and KMS-12 cells following treatment with DTP3 as single agent are from the experiment shown in FIGS. 4 and 5.

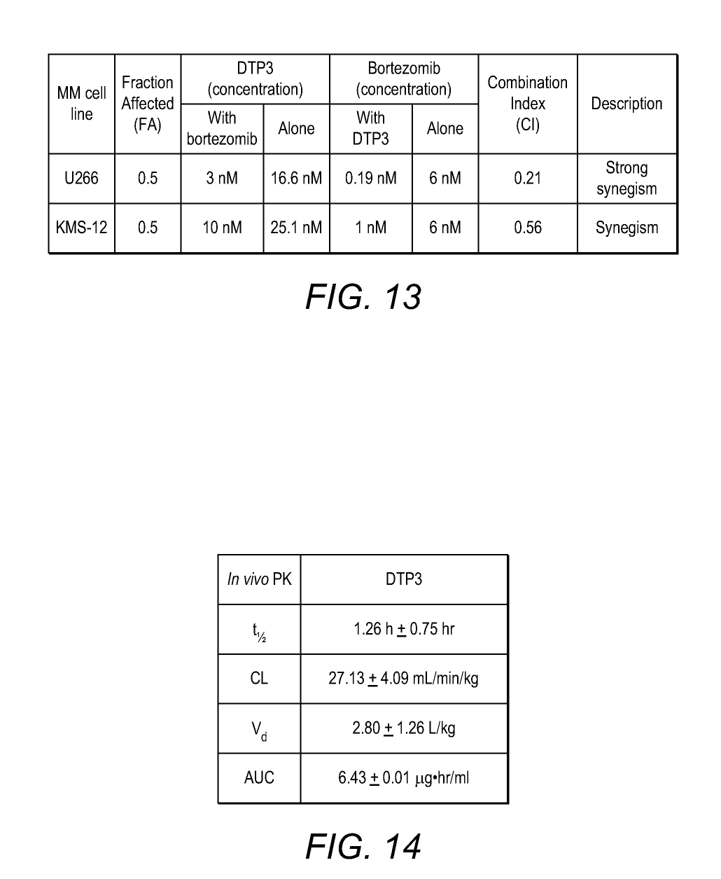

FIG. 13 shows the combination index (CI) of DTP3 and bortezomib for the experiment shown in FIG. 12. Viability data from the experiment shown in FIG. 12 were converted into values representing the fraction of cells affected (FA) equaling 0.5 in the drug-treated cultures compared with untreated cultures, and the interaction of DTP3 with bortezomib was analysed according to the Chou-Talalay method (Chou, 2006). Also shown are the drug concentrations giving the FA value of 0.5 and the description of the CI. Based on the Chou-Talalay equation (Chou, 2006), synergy is present when the CI is less than 1.0 (where values between 0.3 and 0.7 denote synergism, and values between 0.1 and 0.3 denote strong synergism); the combination is additive when CI equals 1.0, and antagonistic when it is more than 1.0 (Chou, 2006).

As shown in FIGS. 12 and 13, DTP3 displayed synergistic activity with bortezomib in two different multiple myeloma cell lines, exhibiting a combination index (CI) of 0.21 (strong synergism) in US266 cells and of 0.56 (synergism) in KNS-12 cells, suggesting it could find indication in the clinic in combination with bortezomib (Chou et al., 2006). These results provide further compelling evidence of the high therapeutic potential of DTP3 in multiple myeloma patients.

### Example 6: The Gadd45β/MKK7 Inhibitor DTP3 has Good Pharmacokinetic Properties

The pharmacokinetic properties of DTP3 were investigated.

FIG. 14 shows pharmacokinetic (PK) values of DTP3 after single intravenous injection at the dose of 10 mg/kg. t_{1/2}, terminal half-life; CL, plasma clearance; V_{d}, volume of distribution; AUC, area under the plasma concentration versus time curve. Values denote means±SD (n=3).

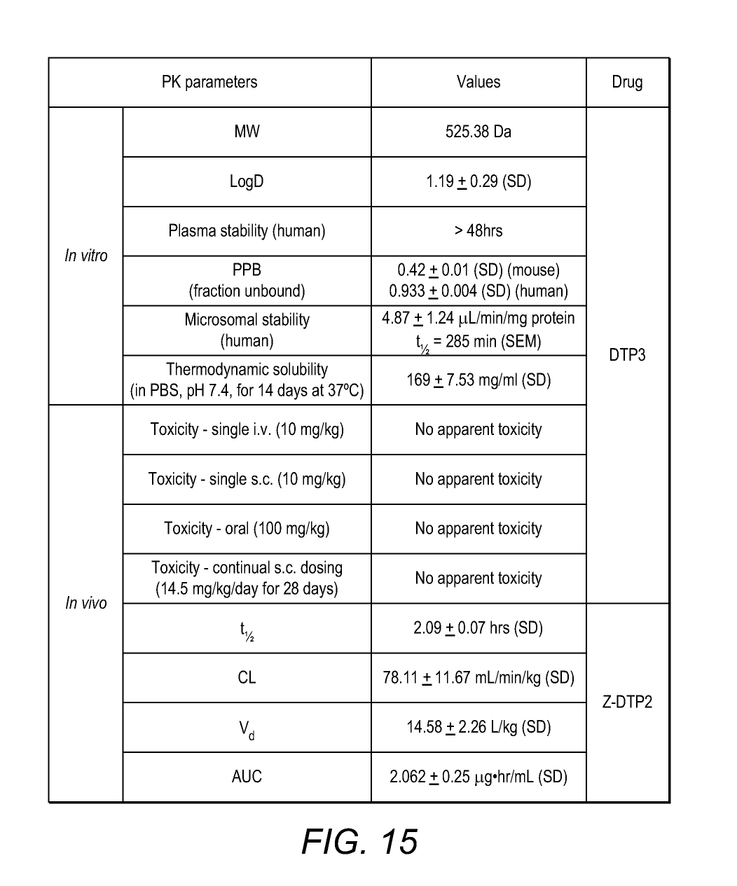

FIG. 15 shows the values of the main in vitro pharmacokinetic (PK) parameters of DTP3, including distribution coefficient (Log D), plasma stability, plasma protein binding (PPB), microsomal stability, and thermodynamic solubility. Also shown is the excellent tolerability of DTP3 in mice after either a single intravenous (i.v.) injection, a subcutaneous (s.c.) injection, or a per os administration at the doses indicated. Additionally, shown are the excellent tolerability and lack of any apparent side effects of DTP3 after prolonged administration via osmotic pumps over a period of 28 days at the therapeutic dose of 14.5 mg/kg/day. Reported at the bottom are the values of the main in-vivo pharmacokinetic parameters of z-DTP2 after a single intravenous injection at the dose of 10 mg/kg, including terminal half-life (t_{1/2}), plasma clearance (CL), volume of distribution (V_{d}), and area under the plasma concentration versus time curve (AUC). DTP3 exhibited somewhat shorter t_{1/2}, but significantly lower CL and much lower V_{d} values than z-DTP2. As a result of these properties, the predicted doses of DTP3 and z-DTP2 required to achieve the therapeutic plasma concentration of 1 μM at the steady state, on the basis of the measured pharmacokinetic values and using the equation described in the Supplemental Experimental Procedures, were 0.81 mg/kg/hr and 3.61 mg/kg/hr, respectively, thus demonstrating the superior pharmacokinetic profile of DTP3 as compared with z-DTP2. Values denote means±SD (n=3) or SEM (n=5), as indicated. MW, molecular weight.

DTP3 showed high aqueous solubility and very high stability in human serum, owing to its resistance to proteolysis, together with a good pharmacokinetic profile and excellent in-vivo tolerability, suitable for a therapeutic purpose. Data for a further Gadd45β/MKK7 inhibitor z-DTP2 were also determined.

### Example 7: The Gadd45β/MKK7 Inhibitor DTP3 is Effective in Xenograft Models in Mice

DTP3 was tested in a plasmacytoma model.

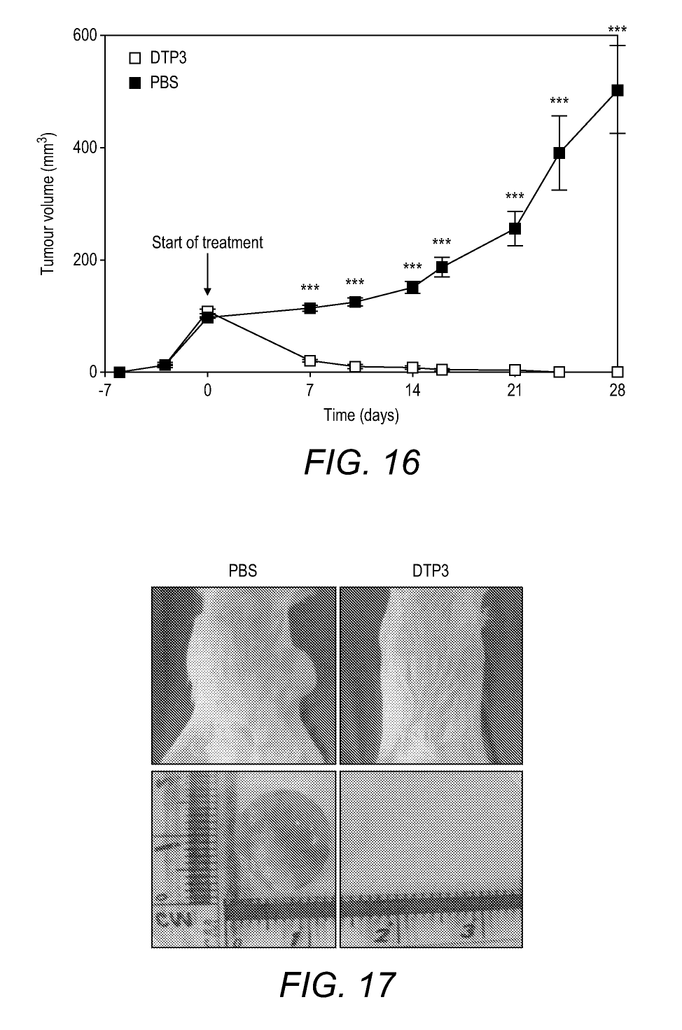

FIG. 16 shows the volumes of subcutaneous U266 myeloma xenografts in mice treated by continual infusion with DTP3 at the dose of 14.5 mg/kg/day or PBS for the times shown. Values denote means±SEM (n=16). ***, p<0.001. FIG. 17 shows images of representative myeloma-bearing mice (top) and isolated tumours (bottom) from the experiment shown in FIG. 16 at day 28.

Remarkably, in the plasmacytoma model, treatment with DTP3 at the dose of 14.5 mg/kg/day virtually eradicated established subcutaneous myeloma xenografts in mice, in the absence of any apparent side effects (see FIGS. 16 and 17). Similar results were obtained in a second plasmacytoma model, generated using a different multiple myeloma cell line (results not shown). At the experimental end-point, on day 28, all the control mice had developed large local tumors, whereas all the mice in the DTP-3 treated cohort had shown a dramatic shrinkage of the tumors (see FIGS. 16 and 17). This therapeutic effect of DTP3 was due to the potent and tumor-selective induction of JNK activation and apoptosis (results not shown), with the appearance of phosphorylated JNK, as early as 24 hr. after the onset of treatment with DTP3, but not with PBS, followed by the appearance, starting on day 3, of caspase-3 and PARP-1 proteolysis products. Coincident with these events, apoptotic cells became evident in the tumor tissue at day 3. As well as the tumour-ablative effects of DTP3, the extent of this JNK-associated, tumor-cell apoptosis markedly increased in magnitude over time.

DTP3 was also tested in an orthotopic xenograft model of multiple myeloma in mice.

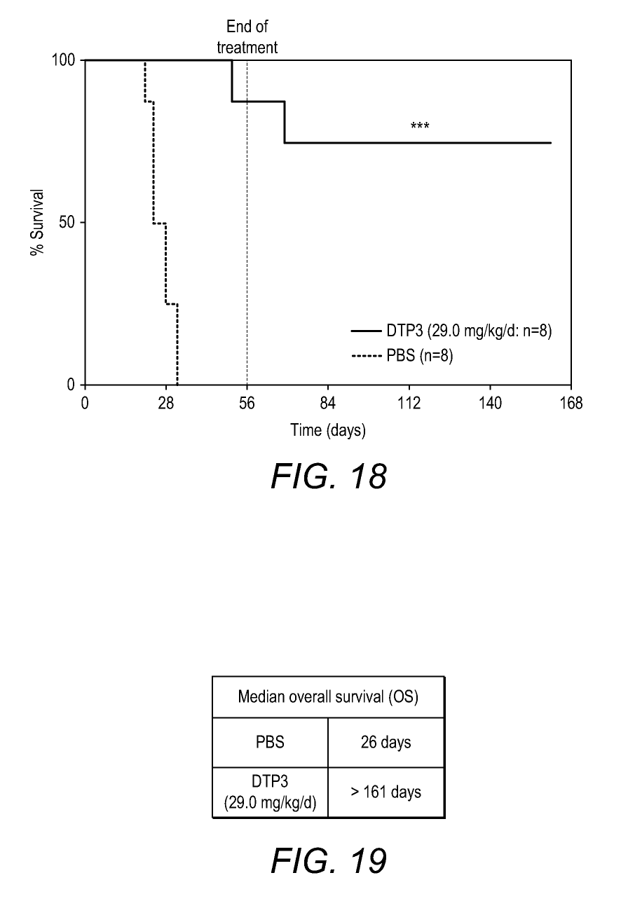

FIG. 18 shows percent survival of mice bearing medullary KMS-12 multiple myeloma xenografts and treated intermittently by infusion with DTP3 at the dose of 29.0 mg/kg/day or PBS (left; n=8, each group) for 8 weeks. FIG. 19 shows the median OS of each animal cohort from the experiment shown in FIG. 18. ***, p<0.0001.

Importantly, DTP3 retained potent anti-cancer activity in the orthotopic xenograft model of MM, which more faithfully recapitulates the human disease. All the control mice developed severe limb paralysis and died within 32 days of treatment start, resulting in a median OS of 26 days (see FIG. 19). Strikingly, DTP3 administration over a period of 8 weeks, at the dose of 29 mg/kg/day, extended the median OS of the mice past the experimental end-point on day 161, without producing any apparent side-effect, this demonstrating the potent therapeutic efficacy of DTP3 against MM, in vivo, and the excellent tolerability of this agent at doses that achieve full therapeutic efficacy.

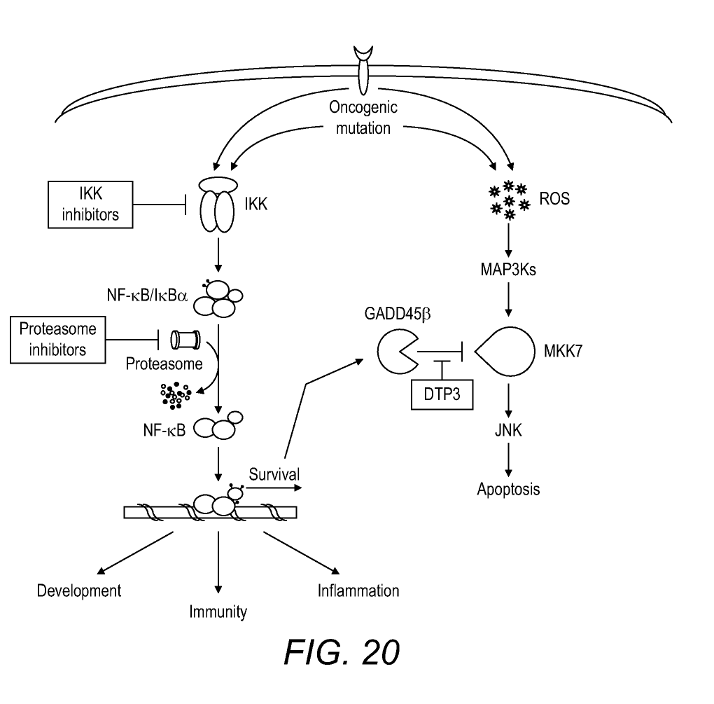

Collectively, these results underscore the potency, safety, and cancer-cell specificity of the pharmacological approach targeting the Gadd45β/MKK7 complex in multiple myeloma (e.g. FIG. 20).

### Example 8: GADD45β and MKK7 are Highly Expressed in Select DLBCL: Cell Lines

Quantitative RT-PCR assays were used to assay the relative levels of mRNA for GADD45 and MKK7 in a panel of genetically heterogeneous DLBCL cell line of both the ABC and GCB subtypes. PMBCs and HEK-293 T-cells and a multiple myeloma cell line were used, respectively, as negative and positive controls.

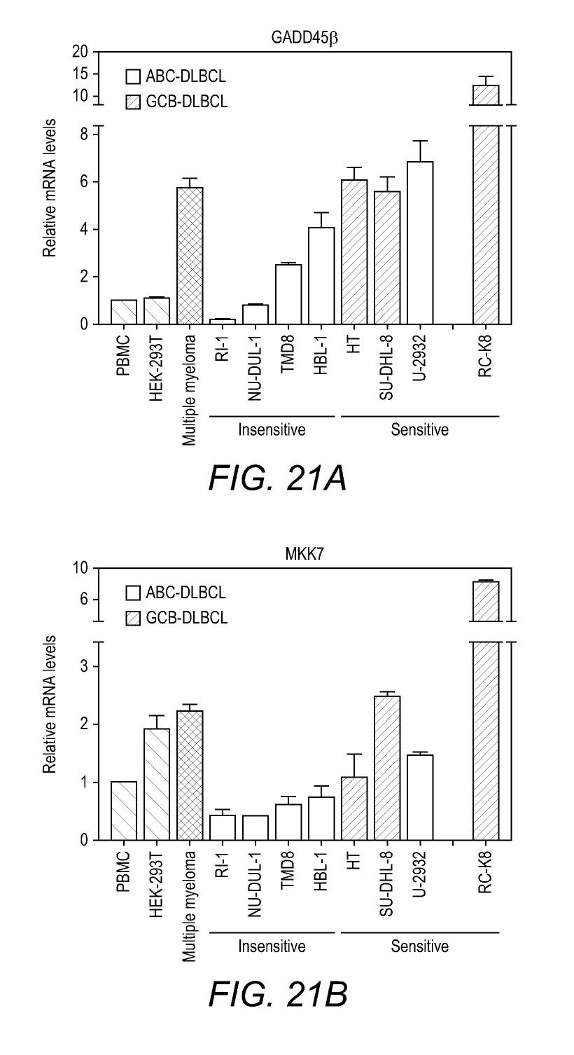

FIG. 21 A shows the GADD45β mRNA levels and FIG. 21 B shows the MKK7 mRNA levels. It can be seen that cell lines known to be sensitive to DTP3 tend to show higher levels of GADD45β mRNA and MKK7 mRNA.

### Example 9: Correlation Between High GADD45β mRNA Expression and Poor Clinical Outcome in Diffuse Large B-Cell Lymphoma (DLBCL) and Hodgkin's Lymphoma (HL) Patients

Overall survival (OS) was measured in newly diagnosed, previously untreated patients with DLBCL or HL. Their levels of GADD45β mRNA expression was measured using the Affymetrix Human Genome U133 Plus 2.0 Array platform, in fresh-frozen biopsy Specimens. DLBCL patients were uniformly treated with R-CHOP-like therapy (i.e. rituximab-containing, anthracycline-based combination chemotherapy), and the series included the 68 patients with this treatment who had available survival information (GSE34171; n=68) (35). HL patients were treated with either ABVD chemotherapy (i.e. doxorubicin, bleomycin, vinblastine, and dacarbazine) or an ABVD-like regimen and, where indicated, radiotherapy (GSE17920; n=130) (36). The relative levels of GADD45β mRNA expression were obtained from the normalised values present in the Oncomine® Research Premium Edition platform. Patients were stratified into two groups in each case on the basis of the GADD45β mRNA levels, using as cut-off the 75th percentile GADD45β expression value as follows: low GADD45β, bottom 75th percentile; high GADD45β top 25th percentile. The OS values of the two cohorts of patients were calculated using the Kaplan-Meier method. The comparisons between the two groups were analysed using the log-rank test.

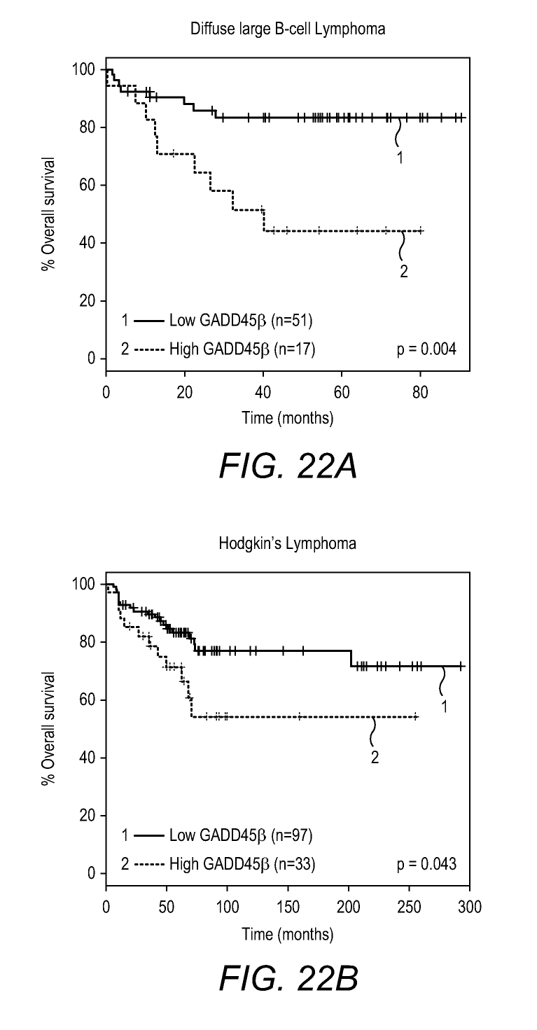

FIG. 22A shows the survival data for the DLBCL cohort and FIG. 22B shows equivalent data for the HL cohort. As can be seen from both cohorts, high GADD45β expression correlates with very-significantly poorer survival.

### Example 10: DTP3 Displays Potent and Selective Therapeutic Activity in DLBCL Cell Lines

A comparison was made between the ability of DTP3 to kill various DLBCL cell lines by apoptosis in vitro. Cell lines HT, SU-DHL-8, U-2932 and RC-K8 which are known to be sensitive to DTP3; and insensitive cell lines RI-1, NU-DUL-1, TMD8, and HBL1 were used. Cell death was assessed using a standard propidium iodide (PI) nuclear staining assay followed by flow cytometry whereby the proportion of cells with a sub G₁-DNA content was measured after cells had been exposed to DTP3 for 6 days at 10 μM. As controls, further populations of cells were left untreated (UT) or were treated with a scrambled D-tripeptide (SCR) for 6 days.

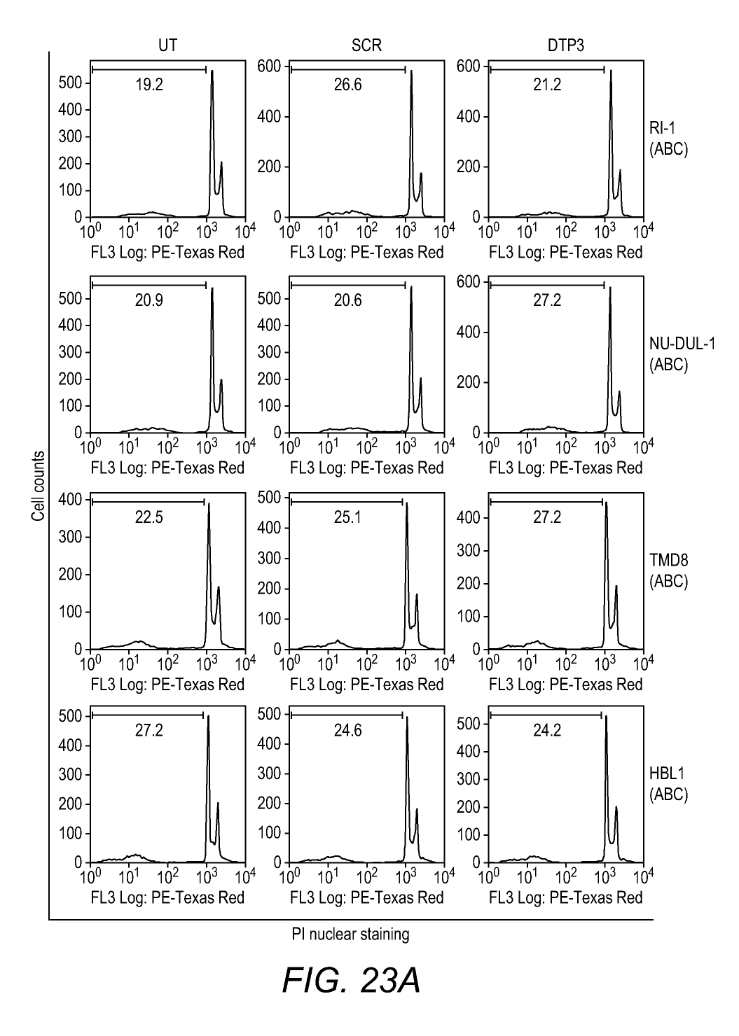

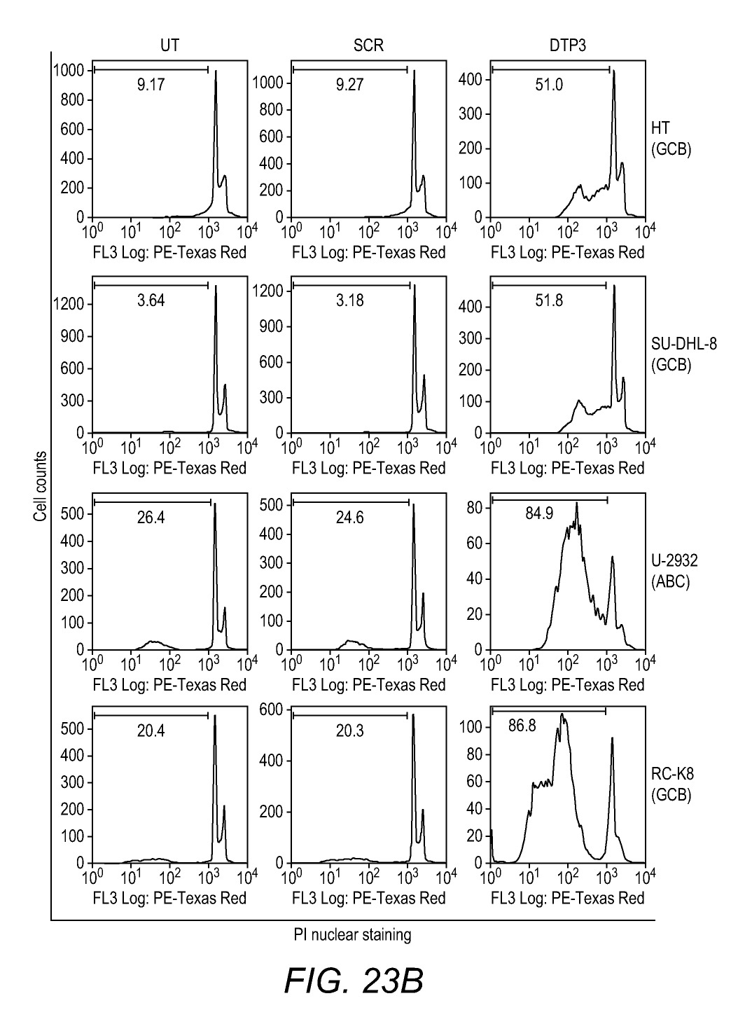

The results are presented in FIG. 23A (insensitive cell lines) and FIG. 23B (sensitive cell lines). They show that the percentage of apoptotic cells in untreated cell and cell treated with the scrambled peptide, is at a baseline level of no more than 27.2%. Insensitive cell lines showed no increase in apoptosis when treated with DTP3. However sensitive cell lines showed substantial increases in apoptosis when treated with DTP3.

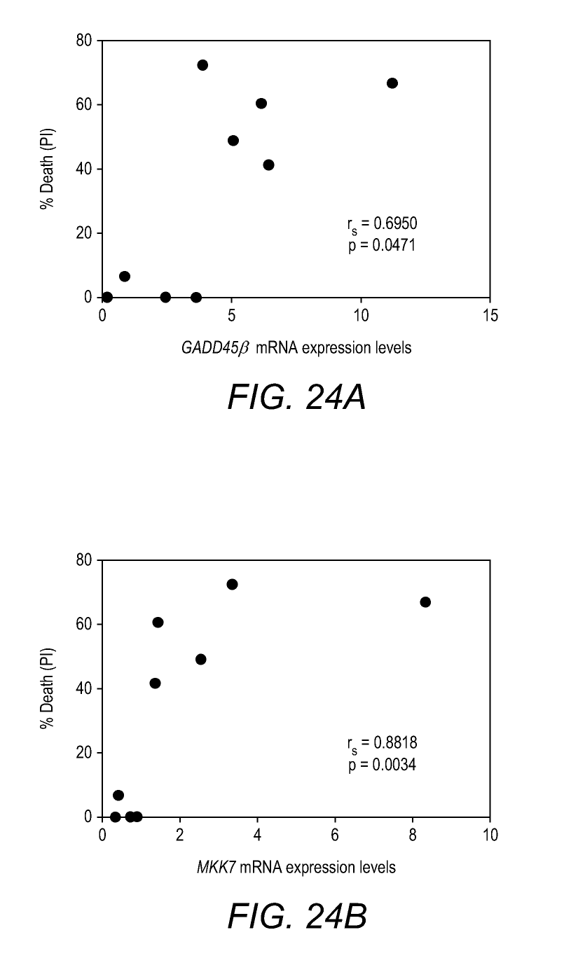

FIG. 24 shows the results of Spearman correlation analysis between relative mRNA levels of Gadd45β (A) and MKK7 (B). mRNA levels are as determined by qRT-PCR, and the percentage of apoptotic cells after treatment with DTP3 (10 μM) are as determined by PI nuclear staining assay in DLBCL cell lines. rₛ=Spearman correlation coefficient. It can be seen that there is a strong correlation between GADD45β mRNA expression and DTP3 induced apoptosis, and between MKK7 mRNA expression and DTP3 induced apoptosis.

### Example 11: Correlation Between DTP3 Therapeutic Activity and GADD45β mRNA Expression in Various Tumour Cell Lines

Relative GADD45β mRNA expression levels for a panel of tumour cell lines having a range of tissues of origin were obtained by qRT-PCR. IC₅₀ values for DTP3 at 144 hrs of 10 μM were also measured for each of the cell lines using a [³H]-thymidine incorporation assay.

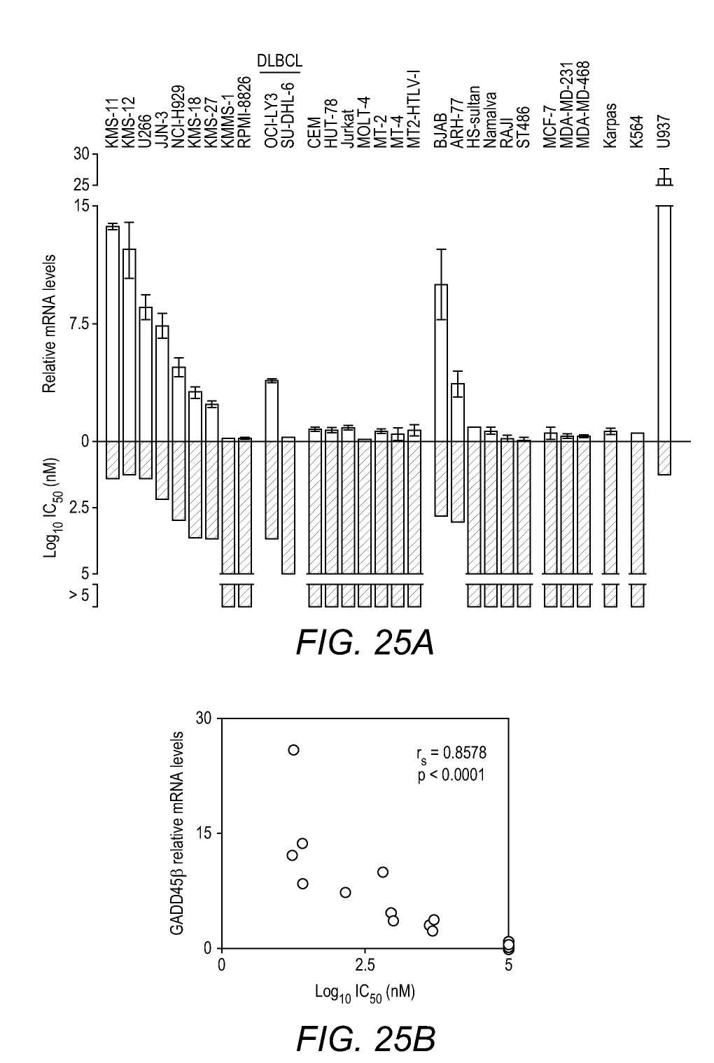

FIG. 25A shows the measured values with the IC₅₀ value expressed as the log₁₀ value. Values denote the mean value from 3 replications (n=3).

FIG. 25B shows a Spearman correlation between the two axis of FIG. 25A where rₛ is the Spearman correlation coefficient. The results show a strong correlation between the two variables and confirm the high target specificity of DTP3 for the GADD45β/MKK7 complex in cells.

### Example 12: Mutational Analysis of the NF-κB Pathway in the DLBCL Cell Lines Tested with DTP3

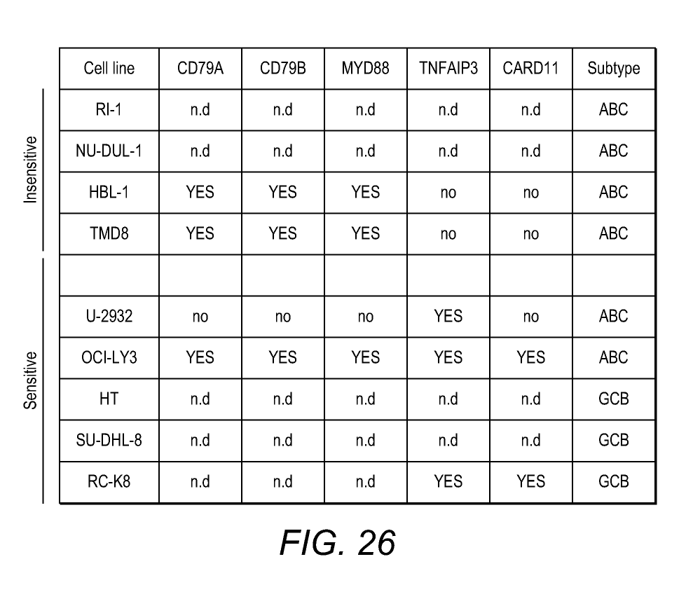

FIG. 26 shows the mutational status of the oncogenic alterations of upstream regulators of the NF-κB pathway most frequently found in primary human DLBCL in the ABC-DLBCL and GCB-DLBCL cell lines that were used to generate the preliminary data shown in this application.

### Experimental Procedures

### Cell Purification and Culture

Cells were cultured according to standard protocols (Mauro et al., 2011; Piva et al., 2008). Additional details are provided in the Supplemental Experimental Procedures.

### Biochemical Assays

Western blots and co-immunoprecipitations were performed as described previously (Papa et al., 2004; Papa et al., 2008; Mauro et al., 2011). Additional details are provided in the Supplemental Experimental Procedures.

### Cellular Assays

[³H]Thymidine incorporation and trypan blue exclusion assays were performed using standard methods (Mauro et al., 2011). IC₅₀ values were defined as the mean concentration of compound inducing 50% inhibition of cell viability relative to the viability in the untreated cultures. Apoptosis analyses were performed using PI staining as described in Mauro et al., 2011. Additional details are provided in the Supplemental Experimental Procedures.

### Peptide Synthesis

Proteins were purified as described in Tornatore et al., 2008. Individual peptides were synthesised as reported in Sandomenico et al., 2012. N-terminal acetylation was carried out by treatment with 10% acetic anhydride in dimethylformamide (DMF; Sigma-Aldrich) containing 5% diisopropyl-ethylamine (DIEA; Sigma-Aldrich). The Z group was introduced by on-resin treatment of the peptides with 0.5 M benzyloxycarbonyl-N-hydroxysuccinimide (Z-OSu; GL-Biochem) in DMF with 5% DIEA. The carboxylic acids used to derivatize the N-terminus of some peptides were pre-activated with 2-(1H-7-azabenzotriazol-1-yl)-1,1,3,3-tetramethyluronium hexafluorophosphate (HATU; GL-Biochem) and DIEA, and subsequently coupled to the resin. Peptides were purified to homogeneity by semi-preparative reverse phase (RP)-HPLC. Peptide identity and purity were determined using liquid chromatography-mass spectrometry (LC-MS). The methods used to assess peptide identity and purity were described previously (Sandomenico et al., 2012; Tornatore et al., 2008). Peptides were then lyophilized and dissolved in dimethylsulphoxide (DMSO; Sigma-Aldrich) at a concentration of 10 mg/mL (stock solution) and stored in aliquots at −20° C. until they were used. IC₅₀ values were defined as the mean concentration of peptide inducing 50% inhibition of GADD45β binding to MKK7 relative to the binding measured in the absence of peptide.

### Animal Studies

Mice were housed in the animal facilities at Hammersmith. All experiments were conducted under PPL 70/6874, after approval by the Imperial College Ethical Review Process and the Home Office. For the plasmacytoma model, NOD/SCID mice (NOD.CB17-Prkdc^{scid}/IcrCrl; Charles River) were injected subcutaneously with 1.0×10⁷ U266 or KMS-11 multiple myeloma cells, and then randomized into treatment groups and treated by infusion, as shown. Tumour volumes were measured as described in Mauro et al., 2011. For the orthotopic multiple myeloma model, mice of the same strain were sub-lethally irradiated and then injected intravenously with 1.0×10⁷ KMS-12-BM multiple myeloma cells, as described in Rabin et al., 2007). Mice were then randomized into treatment groups and treated by infusion for 8 weeks, as shown. Animals were monitored daily and euthanized on day 161 of treatment start or when they reached any of the end-points in the PPL. Additional details are provided in the Supplemental Experimental Procedures.

### Supplemental Experimental Procedures

### Antibodies, Reagents and Western Blots

Western blots were performed as described previously (Papa et al., 2008; Mauro et al., 2011). The antibodies used were: anti-IκBα (sc-1643; 1:5,000), anti-PARP-1 (sc-56197; 1:1,000), anti-β-actin (sc-1616; 1:3,000), anti-MEK-7 (sc-7104; 1:1,000), anti-HA (sc-805; 1:1,000), goat anti-rabbit IgG-HRP (sc-2030; 1:2,500), donkey anti-goat IgG-HRP (sc-2020; 1:5,000), goat anti-mouse IgG-HRP (sc-2005; 1:2,500), anti-Gadd45β (sc-133606; 1:500) (Santa Cruz Biotechnology); anti-PARP-1 (5625P; cleaved product; 1:1,000), anti-caspase-3 (9662S; 1:1,000), anti-JNK (9258S 1:1,000), anti-P-JNK (9251S 1:1,000), anti-p38 (9212; 1:1,000), anti-P-p38 (9215S; 1:1,000), anti-ERK1/2 (4695S; 1:1,000), anti-P-ERK1/2 (4377S; 1:1,000), anti-MKK7 (4172S; 1:1,000), anti-MKK4 (9152S; 1:1,000) (Cell Signalling); anti-RelA (ADI-KASTF110; 1:5,000) (ENZO); anti-FLAG (F-7425; 1:2,000) (Sigma-Aldrich). The anti-FLAG M2 Affinity Gel used for immunoprecipitations and kinase assays was from Sigma-Aldrich. The propidium iodide (PI; 50 μg/mL) and ribonuclease A (50 μg/mL) reagents used for the FACS analysis of apoptosis were also from Sigma-Aldrich. The reagents used for the cell treatments were: human TNFα (2,000 U/mL; Peprotech); 12-O-tetradecanoylphorbol-13-acetate (TPA; 50 ng/mL; Sigma-Aldrich); ionomycin (1 μM; Sigma-Aldrich); IL-6 (10 ng/mL; R&D Systems); IGF-1 (50 ng/mL; R&D Systems); PS-1145 dihydrochloride (20 μM; Sigma-Aldrich); SC-514 (30 μM; Calbiochem); SP600125 (10 μM; Calbiochem); Velcade (bortezomib; ranging from 0.01 nM to 10 μM; Janssen-Cilag).

### Cell Culture and Primary Cell Purification

The human multiple myeloma (KMS-11, KMS-12, KMS-18, KMS-27, U266, JJN-3, NCI-H929, AMO-1, MM1.S, KMM-1, RPMI-8226 [parental]; U266/R10R, MM1/R10R, MM1.R, AMO-1a [drug resistant]), diffuse large B-cell lymphoma (HT, SU-DHL-8, U-2932, RC-K8) cell lines were cultured in RPMI-1640 medium (Invitrogen) supplemented with 10% foetal bovine serum (FBS; Sigma-Aldrich), antibiotics (100 μg/mL penicillin, 100 μg/mL streptomycin), and 1 mM L-glutamine (Invitrogen). Cells were cultured in a humidified incubator in 5% CO₂ at 37° C. The NCI-H929, RPMI-8226, MM1.S and MM1.R cell lines were purchased from the ATCC. The U266 and KMS-12 cell lines were purchased from the Istituto Zooprofilattico “Ugo Umbertini” (Brescia, Italy). The KMS-11, KMS-18, KMS-27, JJN-3, KMM-1 and SU-DHL-6 cell lines were obtained from G. Inghirami (Turin, Italy). The lenalidomide-resistant U266/R10R and MM1/R10R cell lines were kindly provided by R. Orlowski (Houston, Tex., USA) (Bjorklund et al., 2011; Bjorklund et al., 2014). The AMO-1 and bortezomib-resistant AMO-1a cell lines were from the C. Driessen's laboratory (Ruckrich et al., 2009). All the other cell lines were from the G. Franzoso's laboratory.

Primary murine cells were isolated from the spleen or lymph nodes of C57BL/6 mice using standard protocols (Ahmad et al., 2011), after removal of the red blood cells with RBC lysis buffer (Sigma-Aldrich), and then seeded into wells of 96-well plates at a concentration of 6×10⁴ cells/well and cultured in RPMI-1640 medium supplemented with 10% FBS (Sigma-Aldrich), antibiotics (100 μg/mL penicillin and 100 μg/mL streptomycin) and 1 mM L-glutamine (Invitrogen).

### Cell Proliferation, Cell Death, and Apoptosis Assays

[³H]Thymidine incorporation assays were performed using standard protocols (Griffiths and Sundaram, 2011). Briefly, cell lines were seeded into wells of 96-well plates at a concentration of 1.0×10⁴ cells/well and then left untreated or treated daily with the indicated concentrations of peptides and maintained in complete RPMI-1640 medium at 37° C. in 5% CO₂, splitting them with medium as necessary. At 24, 72 or 144 hr, cells were incubated for an additional 16 hr with 0.037 MBq/well of [³H]thymidine (Amersham), and then harvested onto glass fibre filter mats using a 96-well plate automated Tomtec cell harvester (Receptor Technologies) and analysed by liquid scintillation spectroscopy with a LKB Wallac Trilux Microbeta 3 b-counter (PerkinElmer). Values were expressed as the percentage of the counts per minute (cpm) measured in the treated cultures relative to the cpm measured in the respective untreated cultures. The half-maximum inhibitory concentration (IC₅₀) values were calculated using either 5 or 7 concentrations of compound and were defined as the mean concentration of compound inducing 50% inhibition of [³H]thymidine uptake relative to the uptake measured in untreated cells. Trypan blue exclusion assays were performed as reported previously (Mauro et al., 2011; Arnold et al., 2008). Briefly, cells from lentivirus-infected cell lines were seeded into wells of 48-well plates in complete medium at a concentration of 2.0×10⁵ cells/well, and then cultured at 37° C. in 5% CO₂, splitting them as necessary during the assays. Cell viability was monitored over a period of up to 8 days by cell counting using trypan blue, and the numbers of live infected cells in the cultures were extrapolated, where appropriate, from the cell counts by accounting for the percentages of eGFP⁺ cells, using flow cytometry. Values were expressed as the percentage of live infected cells present in the cultures at the times indicated relative to the number of live infected cells present in the same cultures at day 0.

Apoptosis analyses were performed using propidium iodide (PI) nuclear labelling, as described previously (Riccardi and Nicoletti, 2006). Briefly, cells were seeded at a density of 2×10⁵ cells/well into wells of either 24- or 48-well plates and cultured in the presence or absence of treatment, as shown. Cells were then fixed in 70% ice-cold ethanol for 16 hr, washed in PBS, and treated with ribonuclease A (50 μg/mL) for 30 min at room temperature. Samples were finally stained with PI (50 μg/mL) in 0.1% sodium citrate buffer for 2 to 4 hr at 4° C. in the dark, and data were acquired using a FACSCalibur automated system (Becton Dickinson) and analysed with FlowJo.

Splenocytes were treated with Ultra-pure lipopolysaccharide (LPS; 1 ng/mL; ENZO) for 16 hr, and, then, both splenocytes and lymph node cells were treated with D-peptide, PS-1145 or bortezomib for 72 hr. Cell viability was measured by using trypan blue exclusion or [³H]thymidine incorporation assays performed in the manner described above (Mauro et al., 2011; Griffiths and Sundaram, 2011; Arnold et al., 2008), as indicated.

### Drug Synergism

The activities of DTP3 and bortezomib in the U266 and KMS-12 myeloma cell lines, both as single agents and in combination, were determined using [³H]thymidine incorporation assays, performed as described above in “Cell Proliferation, Cell Death, and Apoptosis Assays”. The IC₅₀ values of bortezomib as single agent were determined at 48 hr using 9 concentrations of drug, ranging from 0.1 to 1,000 nM. The IC₅₀ values of DTP3 at day 6 as single agent were from the experiment in FIGS. 4 and 5. The concentrations of DTP3 for the drug interaction studies with bortezomib were selected in pilot experiments. For these studies, U266 and KMS-12 multiple myeloma cells were treated with DTP3 for 6 days at the concentrations of 3 nM and 10 nM, respectively, and bortezomib was added to the cultures at increasing concentrations, ranging from 0.1 to 1,000 nM, 48 hr prior to the measurement of cell viability by [³H]thymidine incorporation. Viability data were converted into values representing the fraction of cells affected (FA) in the drug-treated cultures compared with untreated cultures, and the interaction between DTP3 and bortezomib was analysed using the Chou-Talalay method (Chou, 2006). The combination index (CI) was calculated based upon this method according to the following equation:

CI=(D)₁/(Dx)₁+(D)₂/(Dx)₂

Where (D)₁ and (D)₂ are the doses of drug 1 (DTP3) and drug 2 (bortezomib), respectively, having x effect when used in combination, and (Dx)₁ and (Dx)₂ are the doses of drug 1 and drug 2, respectively, having the same x effect when used alone. Synergy is present when the CI is less than 1.0 (where values between 0.3 and 0.7 denote synergism, and values between 0.1 and 0.3 denote strong synergism); the combination is additive when CI equals 1.0, and antagonistic when it is more than 1.0 (Chou, 2006).

### Co-Immunoprecipitation Assays

Co-immunoprecipitation assays were performed as described previously (Papa et al., 2004). Briefly, human HEK-293T cells were seeded at a density of 1.8-2.2×10⁶ cells/dish in 100 mm² tissue-culture dishes and then transfected with pcDNA-FLAG-hMKK7 and pcDNA-HA-hGADD45β by using the calcium phosphate precipitation method (Papa et al., 2004). 48 hr after transfection, cells were washed with PBS and then lysed in lysis buffer supplemented with protease inhibitors (1 mM phenylmethylsulfonylfluoride, 10 μM chymostatin, 2 μg/mL aprotinin, 2 μg/mL leupeptin; Roche) for 30 min in ice. Cell lysates were cleared by centrifugation at 45,000×g for 40 min and then used for co-immunoprecipitations with anti-FLAG M2 Affinity Gel (Sigma-Aldrich), binding to FLAG-tagged MKK7. Western blots were developed using an anti-HA (binding to HA-hGADD45β) or an anti-MKK7 antibody. For competition assays, the anti-FLAG immunoprecipitates were incubated for 2-4 hr at 4° C. in the presence or absence of bioactive or control inactive D-peptides, before loading them onto SDS-PAGE gels.

The amino acid sequences of the active and inactive control D-peptides used in the co-immunoprecipitation assays were as follows: DTP3, Ac-Tyr-Arg-Phe-NH₂; scrambled (SCRB), Ac-Arg-Phe-Tyr-NH₂. All peptides were in the D configuration.

### Protein Purification and ELISA

Recombinant proteins were purified as described previously (Tornatore et al., 2008). Briefly, full-length human (h)GADD45β was expressed as a protein fused to a poly-histidine (His₆) tag and purified by affinity chromatography using His-Trap columns (GE Healthcare). The His₆-GADD45β protein was used for ELISA analyses. Full-length human (h)MKK7 was expressed as a protein fused to an N-terminal GST tag (GST-MKK7), purified using GST-Trap as above, and then used for ELISA analyses. Purified GST was used as a control in all the experiments. The kinase domain (KD) of hMKK7 (amino acids 101-405) was expressed as a protein fused to an N-terminal His₆ tag in E. coli (BL21[DE3] strain) using the pETDuet-1 vector (Novagen). The resulting His₆-hMKK7-KD protein was purified by affinity chromatography using a His-Trap column, followed by gel filtration, and utilised for CD and MALDI-TOF-MS analyses, following dilution in water or 1 mM ammonium bicarbonate, pH 7.0, as appropriate, from a stock solution prepared at 0.5 mg/mL (13.7 μM) in 25 mM TRIS, 150 mM NaCl buffer at pH 7.0.

The methods used for the ELISA binding and competition assays were described previously (Tornatore et al., 2008). Briefly, His₆-GADD45β was biotinylated using the EZ Link® NHS-LC-biotin kit (Pierce), according to the manufacturer's instructions (Tornatore et al., 2008). Human GST-MKK7 was coated onto wells of 96-well plates by incubation at 42 nM in ELISA buffer (25 mM Tris pH 7.5, 150 mM NaCl, 1 mM DTT, 1 mM EDTA) for 16 hr at 4° C., followed by blocking with 2% non-fat dry milk in PBS. Biotin-labelled His₆-hGADD45β was then added to the wells at increasing concentrations ranging from 5.2 to 168 nM and incubated in the dark for 1 hr at 37° C., followed by washing with Tween (T)-PBS. Bound His₆-hGADD45β was revealed using horseradish peroxidase (HRP)-conjugated streptavidin (1:1,000) and the HRP chromogenic substrate, o-phenylenediamine (OPD). GST-coated wells were used as controls.

### Pharmacokinetic Analyses

In vitro pharmacokinetic assays were performed according to established, in-house protocols and included: plasma stability (human), thermodynamic solubility (PBS pH 7.4, at 37° C. for 14 days), microsomal stability (human), plasma protein binding (PPB; mouse and human), and distribution coefficient (Log D). In vivo pharmacokinetic analyses were also performed at Cyprotex according to established, in-house protocols. Briefly, the peptides were dissolved in PBS and administered by single intravenous injection to CD1 male mice at the dose of 10 mg/kg (25-30 g of total body weight; n=3 mice per time point per compound). Blood samples were collected in heparinized tubes at up to 7 time points after this injection (i.e. 0.08, 0.25, 0.5, 1, 2, 4 and 8 hr), and the plasma concentrations of the test peptide were determined at each time point by LC-MS/MS, using a five-point standard curve covering a peptide concentration range from 3 to 3,000 ng/mL. Standard curves were prepared using blank plasma matrices and treated in the same manner as the test samples. The main pharmacokinetic parameters determined in these analyses were: terminal half-life (t_{1/2}), plasma clearance (CL), volume of distribution (V_{d}), and area under the plasma concentration versus time curve (AUC) (FIGS. 14 and 15). Values were calculated from the data of the peptide plasma concentration versus time curves using a non-compartmental model analysis, as described elsewhere (Groulx, 2006; Landaw and Di Stefano 3^{rd}, 1982). The doses of z-DTP2 and DTP3 predicted to achieve the therapeutic plasma concentration of 1 μM at the steady state were calculated from the measured pharmacokinetic values using the following equation: k₀=k₁×V_{d}×Cₚ (where V_{d} is expressed in L/kg; Cₚ is the plasma concentration at the steady state expressed in mg/L; k₀ is the zero order input expressed in mg/kg/hr; k₁ is the first order elimination constant [0.693/t_{1/2}]). The calculated k₀ values of DTP3 and z-DTP2 on the basis of this equation were 0.81 mg/kg/hr and 3.61 mg/kg/hr, respectively. The measured plasma concentration of DTP3 after continual administration via osmotic pumps was very similar to the predicted plasma concentration of 1 μM.

### Mouse Xenografts

Mice were housed in the animal facilities at the Hammersmith Campus, Imperial College London, and experiments were conducted under the Home Office Authority (PPL 70/6874). The PPL was reviewed by the Imperial College Ethical Review Process (ERP) and the Home Office. For experiments in the subcutaneous xenograft models (FIGS. 16-17), 6 to 8-week old male NOD/SCID mice (NOD.CB17-Prkdc^{scid}/IcrCrl; Charles River) were injected intraperitoneally with anti-CD122 monoclonal antibody (TM-β1 clone; 200 μg/mouse) (Tanaka et al., 1991) and, 24 hr later, subcutaneously in the left flank with 1.0×10⁷ U266 or KMS-11 human multiple myeloma cells resuspended in 200 μL of sterile PBS and Matrigel (1:1) (BD Biosciences), using a 1-mL Luer-Lok™ Tip syringe (BD Biosciences) with a 25-gauge needle (Bhutani et al., 2007). U266 and KMS-11 cells were harvested from exponentially growing cultures, washed once with serum-free medium, and resuspended in PBS immediately before injection (Bhutani et al., 2007). The anti-CD122 antibody was affinity purified using HiTrap Protein G HP columns (GE Healthcare), according to the manufacturer's instructions. One week after injection of the cells, when tumour volumes had reached approximately 100 mm³ (U266 cells) or tumours had yet to be palpable (KMS-11 cells), mice were randomized into treatment groups (n=16 or n=4 per group, respectively; day 0) (Bhutani et al., 2007) and, then, treated by continual infusion of DTP3 at a dose of 14.5 mg/kg/d, yielding a plasma concentration of 0.70±0.09 μM (SEM; n=16), or PBS. Treatments were administered using Alzet osmotic pumps (model 1004; 28-day delivery at the rate of 0.11 mL/hr; Charles River), implanted surgically subcutaneously into the right flank of each mouse, according to the manufacturer's instructions (Gullapalli et al., 2012). For systemic administration, DTP3 was dissolved at the concentration of 147 mg/mL in PBS, and mice were euthanized on day 28 of the start of treatment with CO₂. Tumour diameters were measured at the indicated times using a vernier calliper, and volumes were estimated from calliper measurements using the following equation: volume=A×B²/2 (where A is the larger diameter, and B is the smaller diameter of the tumour) (Mauro et al., 2011). Results were reproduced at least twice. The plasma concentration of DTP3 was measured at the steady state by LC-MS/MS at Cyprotex, as described above.

For experiments in the medullary xenograft model, 6 to 8-week old male NOD/SCID mice (NOD.CB17-Prkdc^{scid}/IcrCrl; Charles River) were sub-lethally irradiated (3.50 Gy) and, then, injected intraperitoneally with purified anti-CD122 monoclonal antibody (200 μg/mouse), as above. 24 hr later, mice were injected intravenously with KMS-12-BM human multiple myeloma cells (1.0×10⁷ cells/mouse) resuspended in 100 μL of PBS, as described previously (Rabin et al., 2007). As for subcutaneous xenografts, KMS-12-BM cells were harvested from exponentially growing cultures, washed once with serum-free medium, and resuspended in PBS immediately before the injection into mice. Three days after injection of the cells, mice were randomized into two treatment groups (n=8 per group; day 0). Treatments were maintained for a period of 8 weeks, and the experiment was stopped on day 161 of treatment start. DTP3 was administered intermittently by infusion using Alzet osmotic pumps (model 1002; 14-day delivery at the rate of 0.25 mL/hr; Charles River) implanted subcutaneously, as described above, two-week on/two-week off, at the dose of 29.0 mg/kg/day. PBS in the control group was also administered for 8 weeks. Animals were monitored daily for weight loss and other clinical signs of medullary disease, such as hind limb paralysis and behavioural changes, and euthanized with CO₂ on day 161 of treatment start or when the disease severity reached any of the end-points stated in the PPL. Results were reproduced at least twice.

### Statistical Analyses

Results were expressed as the mean of the indicated number of samples. The statistical error was calculated as either standard deviation (SD) or standard error of the mean (SEM), as indicated, according to the typology of the experiments. Unless otherwise stated, the statistical significance of the values was determined using the Student's t-test. A value of p<0.05 was considered statistically significant. The median OS for the cohorts of tumour-bearing mice shown in FIG. 19 was calculated using GraphPad software, and comparisons between the cohorts were analysed using the log-rank test. The sample size for the analyses shown was not pre-determined. No samples or animals were excluded from the analyses. No specific method of randomization was used for the animal studies. The animal experiments were not blinded.

### REFERENCES

Ahmad, F., et al. Activation of anti-tumour immune response and reduction of regulatory T cells with Mycobacterium indicus pranii (MIP) therapy in tumour bearing mice. PLoS One 6, e25424-e25424 (2011).
Arnold, A. A., et al. Preclinical studies of Apogossypolone: a new nonpeptidic pan small-molecule inhibitor of Bcl-2, Bcl-x_{L} and Mcl-1 proteins in Follicular Small Cleaved Cell Lymphoma model. Mol. Cancer. 14, 7-20 (2008).
Bhutani, M., et al. Capsaicin Is a Novel Blocker of Constitutive and Interleukin-6-Inducible STAT3 Activation. Clin. Cancer Res. 13, 3024-3032 (2007).
Bjorklund, C. C., Ma, W., Wang, Z. Q., Davis, R. E., Kuhn, D. J., Kornblau, S. M., Wang, M., Shah, J. J., and Orlowski, R. Z. (2011). Evidence of a role for activation of Wnt/beta-catenin signaling in the resistance of plasma cells to lenalidomide. J. Biol. Chem. 286, 11009-11020.
Bjorklund, C. C., Baladandayuthapani, V., Lin, H. Y., Jones, R. J., Kuiatse, I., Wang, H., Yang, J., Shah, J. J., Thomas, S. K., Wang, M., et al. (2014). Evidence of a role for CD44 and cell adhesion in mediating resistance to lenalidomide in multiple myeloma: therapeutic implications. Leukemia 28, 373-383.
Chou, T. C. (2006). Theoretical basis, experimental design, and computerized simulation of synergism and antagonism in drug combination studies. Pharmacol. Rev. 68, 621-681.
Di Donato, J. A., Mercurio, F., and Karin, M. (2012). NF-κB and the link between inflammation and cancer. Immunol. Rev. 246, 379-400.
Greipp, P. R., et al. International staging system for multiple myeloma. J. Clin. Oncol. 23, 3412-3420 (2005).
Griffiths, M., and Sundaram, H. Drug design and testing: profiling of antiproliferative agents for cancer therapy using a cell-based methyl-[³H]-thymidine incorporation assay. Methods Mol. Biol. 731, 451-65 (2011).
Groulx, A. Introduction to Pharmacokinetics. Scian. News. 9, 1-5 (2006).
Gullapalli, R., et al. Development of ALZET® osmotic pump compatible solvent compositions to solubilize poorly soluble compounds for preclinical studies. Drug Deliv. 19, 239-246 (2012).
Landaw, E. M., and Di Stefano 3^{rd}, J. J. Multiexponential, multicompartmental, and noncompartmental modeling. II. Data analysis and statistical considerations. Am. J. Physiol. Regul. Integr. Comp. Physiol. 243, 1-6 (1982).
Mahindra, A., Laubach, J., Raje, N., Munshi, N., Richardson, P. G., and Anderson, K. (2012). Latest advances and current challenges in the treatment of multiple myeloma. Nat. Rev. Clin. Oncol. 9, 135-143.
Mauro, C., Leow, S. C., Anso E., Rocha, Thotakura, A. K., Tornatore, Moretti, M., De Smaele, E., Tergaonkar, A. B. V., et al. (2011). NF-κB controls energy homeostasis and metabolic adaptation by upregulating mitochondrial respiration. Nat. Cell Biol. 13, 1272-1279.
Papa, S., Monti, S. M., Vitale, R. M., Bubici, C., Jayawardena, S., Alvarez, K., De Smaele, E., Dathan, N., Pedone, C., Ruvo, M., and Franzoso, G. (2007). Insights into the structural basis of the GADD45β-mediated inactivation of the JNK kinase, MKK7/JNKK2. J Biol. Chem. 282, 19029-19041.
Papa, S., Zazzeroni, F., Bubici, C., Jayawardena, S., Alvarez, K., Matsuda, S., Nguyen, D. U., Pham, C. G., Nelsbach, A. H., Melis, T., et al. (2004). GADD45β mediates the NF-□B suppression of JNK signalling by targeting MKK7/JNKK2. Nat. Cell Biol. 6, 146-153.
Papa, S., Zazzeroni, F., Fu, Y. X., Bubici, C., Alvarez, K., Dean, K., Christiansen, P. A., Anders, R. A., and Franzoso, G. (2008). GADD45β promotes hepatocyte survival during liver regeneration in mice by modulating JNK signaling. J Clin. Invest. 118, 1911-1923.
Piva, R., Ruggeri, B., Williams, M., Costa, G., Tamagno, I., Ferrero, D., Giai, V., Coscia, M., Peola, S., Massaia, M., et al. (2008). CEP-18770: A novel, orally active proteasome inhibitor with a tumor-selective pharmacologic profile competitive with bortezomib. Blood 111, 2765-2775.
Rabin, N., Kyriakou, C., Coulton, L., Gallagher, O. M., Buckle, C., Benjamin, R., Singh, N., Glassford, J., Otsuki, T., Nathwani, A. C., et al. (2007). A new xenograft model of myeloma bone disease demonstrating the efficacy of human mesenchymal stem cells expressing osteoprotegerin by lentiviral gene transfer. Leukemia 21, 2181-2191.
Rajkumar, S. V. (2011). Treatment of multiple myeloma. Nat. Rev. Clin. Oncol. 8, 479-491.
Riccardi, C., and Nicoletti, I. Analysis of apoptosis by propidium iodide staining and flow cytometry. Nat. Protoc. 1, 1458-1461 (2006).
Richardson, P. G. (2010). Improving the therapeutic index in myeloma. Blood 116, 4733-4734.
Robinson, S. C., et al. A chemokine receptor antagonist inhibits experimental breast tumor growth. Cancer Res. 63, 8360-8365 (2003).
Ruckrich, T., Kraus, M., Gogel, J., Beck, A., Ovaa, H., Verdoes, M., Overkleeft, H. S., Kalbacher, H., and Driessen, C. (2009). Characterization of the ubiquitin-proteasome system in bortezomib-adapted cells. Leukemia 23, 1098-1105.
Sandomenico, A., Russo, A., Palmieri, G., Bergamo, P., Gogliettino, M., Falcigno, L., and Ruvo, M. (2012). Small peptide inhibitors of Acetyl-Peptide Hydrolase having an uncommon mechanism of inhibition and a stable bent conformation. J. Med. Chem. 55, 2102-2111.
Sandomenico, A., et al. A comparative structural and bioanalytical study of IVIG clinical lots. Mol. Biotechnol. 54, 983-95 (2013).
Tanaka, T., et al. A novel monoclonal antibody against murine IL-2 receptor beta-chain. Characterization of receptor expression in normal lymphoid cells and EL-4 cells. J. Immunol. 147, 2222-2228 (1991).
Tornatore, L., Marasco, D. Dathan, N., Vitale, R. M., Benedetti, E., Papa, S., Franzoso, G., and Ruvo, M. (2008). GADD45β forms a homodimeric complex that binds tightly to MKK7. J. Mol. Biol. 378, 97-111.
Yang, H., et al. TNF-inhibits asbestos-induced cytotoxicity via a NF-κB-dependent pathway, a possible mechanism for asbestos-induced oncogenesis. Proc. Natl. Acad. Sci. USA 103, 10397-10402 (2006).
Yang, J., et al. Anti-b2-microglobulin monoclonal antibodies induce apoptosis in myeloma cells by recruiting MHC class I to and excluogrowth and survival cytokine receptors from lipid rafts. Blood. 110, 3028-35. (2007).

1.-68. (canceled)

69. A method of treating a resistant haematological malignancy in a subject, the method comprising administering a therapeutically effective amount of a Gadd45β/MKK7 inhibitor to the subject.

70. The method of claim 69, wherein the Gadd45β/MKK7 inhibitor is a compound of formula I:
X₁-A-X₂ I:
wherein,
A is A″″,
or A″-[M-A′-]ₙM-A′″;
A″″ is A″,
A′″,
or Z₁—Y₂—Y₃—Z₄, wherein Y₂-Y₃ is an oligopeptide moiety or an oligopeptoid moiety having the residues Y₂-Y₃ and Z₁ is attached to the N-terminal nitrogen of Y₂-Y₃ and Z₄ is attached to the C-terminal carbon of Y₂-Y₃;
A″ is A′,
or Y₁-Y₂—Y₃—Z₄, wherein Y₁-Y₂—Y₃ is an oligopeptoid moiety or an oligopeptoid moiety comprising the residues: Y₁-Y₂—Y₃ and Z₄ is attached to the C-terminal carbon of Y₁-Y₂—Y₃;
A′″ is A′,
or Z₁—Y₂-Y₃—Y₄, wherein Y₂-Y₃-Y₄ is an oligopeptoid moiety or an oligopeptoid moiety comprising the residues Y₂-Y₃—Y₄ and Z₁ is attached to the N-terminal nitrogen of Y₂-Y₃—Y₄;
each occurrence of A′ is independently an oligopeptide moeity or an oligopeptoid moiety comprising the residues Y₁-Y₂—Y₃—Y₄;
n is an integer from zero to 18;
Y₁ and Y₄ are independently amino acid residues or residues of amino acid derivatives having aromatic side chains;
Y₂ is an amino acid residue or a residue of an amino acid derivative or is absent;
Y₃ is an amino acid residue or a residue of an amino acid derivative or is absent;
Z₁ is a group of formula IL 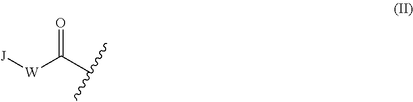
which is linked to the N-terminal nitrogen of Y₂,
W is absent, or an oxygen, or a nitrogen, or an alkylene group of from one to three carbons,
which alkylene group of from one to three carbons is optionally substituted by at least one substituent selected from alkyl of from one to four carbons, or 5-10 membered carbocyclic or heterocyclic aromatic group;
J is a 5-10 membered carbocyclic or heterocyclic aromatic group,
which aromatic group is optionally substituted by at least one substituent selected from hydroxyl, halogen, alkyl of from one to four carbons, or alkoxy of from one to four carbon atoms;
Z₄ represents a group of formula III: 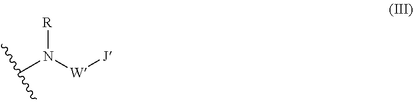
which is linked to the C-terminal carbon of Y₃,
R is hydrogen or alkyl of from one to four carbons;
W′ is absent or an alkylene group of from one to three carbons;
which alkylene group of from one to three carbons is optionally substituted by at least one substituent selected from alkyl of from one to four carbons, or 5-10 membered carbocyclic or heterocyclic aromatic group;
J′ is a 3-10 membered aliphatic carbocyclic group or a 5-10 membered carbocyclic or heterocyclic aromatic group;
which aliphatic or aromatic group is optionally substituted by at least one substituent selected from hydroxyl, halogen, alkyl of from one to four carbons, or alkoxy of from one to four carbon atoms;
M is a peptide bond between preceding oligopeptide or oligopeptoid moiety (A′, A″ or A′″) and following oligopeptide or oligopeptide moiety (A′, A″ or A′″) or a linker moiety attached via an amide bond, an ester bond, an ether bond, or a thioether bond to the terminal carboxylic group of preceding oligopeptide or oligopeptoid moiety (A′, A″ or A′″) and via an amide bond, an ester bond, an ether bond, or a thioether bond to the terminal amino group of following oligopeptoid moiety (A′, A″ or A′″);
X₁ is absent, or is a moiety added to the amino terminal of A in order to block the free amino group;
X₂ is absent or is a moiety added to the carboxyl terminal of A in order to block the free carboxyl group;
with the proviso that X₁ is absent if A comprises Z₁ and X₂ is absent if A comprises Z₄;
or derivatives thereof, said derivatives being selected from the group consisting of:
a) oligomers or multimers of molecules of the compound of formula I, said oligomers and multimers comprising two or more molecules of the compound of formula I each linked to a common scaffold moiety via an amide bond formed between an amino or carboxylic acid group present in molecules of the compound of formula I and an opposite amino or carboxylic acid group on a scaffold moiety said scaffold moiety participating in at least 2 amide bonds,
b) derivatives comprising a molecule of the compound of formula I or an oligomer or multimer thereof as defined above in part a) conjugated via an amide bond, an ester bond, an ether bond or a thioether bond to:
PEG,
PEG-based compounds,
cell-penetrating peptides,
fluorescent dyes,
biotin or other tag moiety,
fatty acids,
nanoparticles of discrete size
or chelating ligands complexed with metallic or
radioactive ions.
c) derivatives comprising a molecule of the compound of formula I or an oligomer or multimer thereof as defined in part a) which has been modified by amidation, glycosylation, carbamylation, acylation, sulfation, phosphorylation, cyclization, lipidisation, pegylation or linkage to a peptide or peptoid fusion partner to make a fusion peptide or fusion peptoid.
and
d) salts and solvates of a molecule of the compound of formula I or of a derivative thereof as defined in part a) orb) above.

71. The method of claim 70, wherein the Gadd45β/MKK7 inhibitor is DTP3, or a derivative thereof, or a salt thereof including a salt of a derivative thereof.

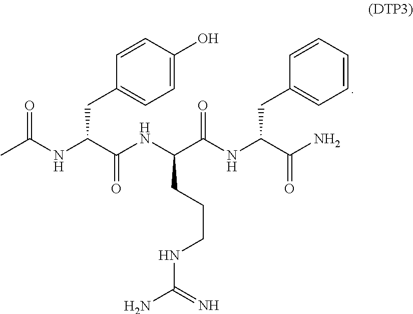 [SMILES: CC(=O)N[C@H](Cc1ccc(O)cc1)C(=O)N[C@H](CCCNC(=N)N)C(=O)N[C@H](Cc1ccccc1)C(N)=O]

72. The method of claim 69, wherein the resistant haematological malignancy is multiple myeloma which is resistant to treatment with an anti-cancer agent selected from the group consisting of a proteasome inhibitor, an IMiD anti-cancer agent and a glucocorticoid, or is resistant to treatment with bortezomib, or one or more of lenalidomide, thalidomide and pomalidomide, or dexamethasone.

73. The method of claim 69, wherein the resistant haematological malignancy is diffuse large B-cell lymphoma (DLBCL) which is resistant to treatment with an anti-cancer agent selected from the group consisting of an anti-CD20 agent, a cytotoxic agent, an anthracycline antibiotic anti-cancer agent, a mitotic inhibitor, a glucocorticoid and a Bruton's tyrosine kinase inhibitor.

74. The method of claim 72, wherein the resistant haematological malignancy is diffuse large B-cell lymphoma (DLBCL) which is resistant to treatment with rituximab, or cyclophosphamide, or doxorubicin, or vincristine, or prednisone, or ibrutinib.

75. The method of claim 69, wherein the resistant haematological malignancy is Hodgkin's Lymphoma which is resistant to treatment with an anti-cancer agent, or vincristine, or doxorubicin, or dexamethasone, or bleomycin, or etoposide.

76. The method of claim 69, wherein the Gadd45β/MKK7 inhibitor administered in combination with another anti-cancer agent.

77. The method of claim 69 further comprising:
providing a subject having a haematological malignancy;
determining whether the haematological malignancy is a resistant haematological malignancy; and
if the haematological malignancy is determined to be a resistant haematological malignancy, administering the Gadd45β/MKK7 inhibitor to the subject.

78. The method of claim 69, wherein, prior to administration of the Gadd45β/MKK7 inhibitor to the subject, a course of a different therapeutic agent indicated for said haematological malignancy is administered to the subject.

79. The method of claim 76, wherein the further anti-cancer agent is bortezomib, or vincristine, or doxorubicin, or bleomycin, or etoposide, or is selected from the group consisting of lenalidomide, thalidomide and pomalidomide, or is dexamethasone.

80. The method of claim 76, wherein the resistant haematological malignancy is multiple myeloma, or Hodgkin's lymphoma, or DLBCL.

81. The method of claim 76, wherein the Gadd45β/MKK7 inhibitor is
i) DTP3, or a derivative thereof, or a salt thereof including a salt of a derivative thereof 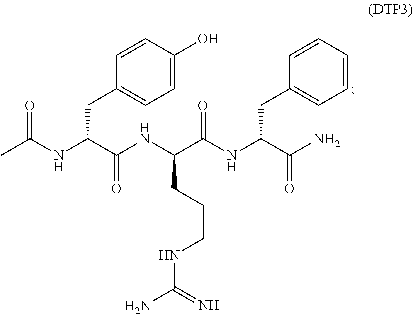 [SMILES: CC(=O)N[C@H](Cc1ccc(O)cc1)C(=O)N[C@H](CCCNC(=N)N)C(=O)N[C@H](Cc1ccccc1)C(N)=O]
and further comprising administering a therapeutically effective amount of
ii) a further anti-cancer agent which is a glucocorticoid, or a proteasome inhibitor, or an IMiD anti-cancer agent to the subject.

82. The method of claim 81, wherein the DTP3 or the derivative and/or the salt thereof and the further anti-cancer agent are administered simultaneously, sequentially or separately.

83. (canceled)

84. (canceled)

85. The method of claim 69, wherein the resistant haematological malignancy is multiple myeloma and the Gadd45β/MKK7 inhibitor is
i) DTTP3, or a derivative thereof, or a salt thereof including a salt of a derivative thereof 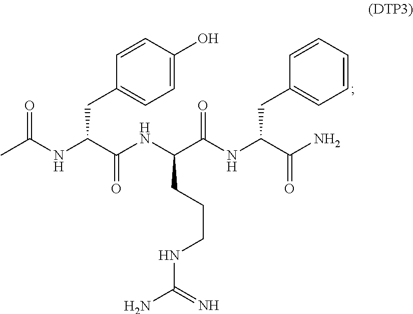 [SMILES: CC(=O)N[C@H](Cc1ccc(O)cc1)C(=O)N[C@H](CCCNC(=N)N)C(=O)N[C@H](Cc1ccccc1)C(N)=O]
and further comprising administering a therapeutically effective amount of
ii) a further anti-cancer agent which is a proteasome inhibitor, or an IMiD anti-cancer agent, or a glucocorticoid, or a proteasome inhibitor.

86. The method of claim 69, wherein the resistant haematological malignancy is DLBCL and the Gadd45β/MKK7 inhibitor is
i) DTP3, or a derivative thereof, or a salt thereof including a salt of a derivative thereof 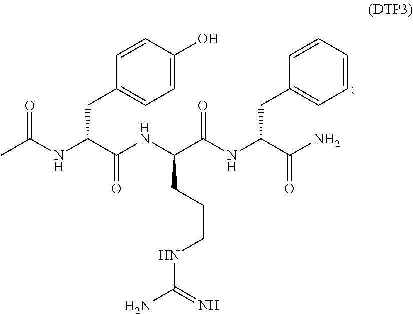 [SMILES: CC(=O)N[C@H](Cc1ccc(O)cc1)C(=O)N[C@H](CCCNC(=N)N)C(=O)N[C@H](Cc1ccccc1)C(N)=O]
and further comprising administering a therapeutically effective amount of
ii) a further anti-cancer agent which is a glucocorticoid, or ibrutinib, or doxorubicin, or vincristine, or rituximab, or cyclophosphamide.

87. The method of claim 69, wherein the resistant haematological malignancy is Hodgkin's lymphoma and the Gadd45β/MKK7 inhibitor is
i) DTP3, or a derivative thereof, or a salt thereof including a salt of a derivative thereof 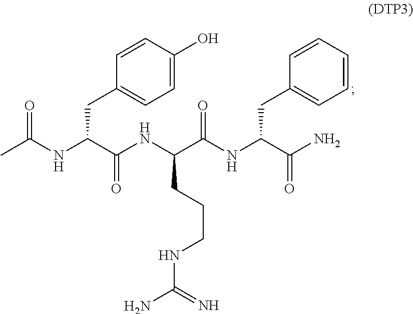 [SMILES: CC(=O)N[C@H](Cc1ccc(O)cc1)C(=O)N[C@H](CCCNC(=N)N)C(=O)N[C@H](Cc1ccccc1)C(N)=O]
and further comprising administering a therapeutically effective amount of
ii) a further anti-cancer agent which is vincristine, or doxorubicin, or bleomycin, or etoposide.
